(19) United States
(12) Patent Application Publication
(10) Pub. No.: US 2024/0000846 A1
(43) Pub. Date: Jan. 4, 2024

(54) COMPOSITIONS AND METHODS FOR TREATING HEMATOPOIETIC MALIGNANCY

(71) Applicant: VOR BIOPHARMA INC., Cambridge, MA (US)

(72) Inventors: Christopher Slapak, Cambridge, MA (US); Glen Raffel, Cambridge, MA (US); Michelle Lin, Cambridge, MA (US); John Lydeard, Cambridge, MA (US); Tirtha Chakraborty, Cambridge, MA (US)

(73) Assignee: VOR BIOPHARMA INC., Cambridge, MA (US)

(21) Appl. No.: 18/033,461

(22) PCT Filed: Oct. 27, 2021
(86) PCT No.: PCT/US2021/056885

§ 371 Date: Apr. 24, 2023

Related U.S. Application Data
(60) Provisional application No. 63/165,950, filed on Mar. 25, 2021.
(60) Provisional application No. 63/106,136, filed on Oct. 27, 2020.

(51) Int. Cl. A61K 35/28; A61K 47/68; A61K 45/06; C12N 5/0789; C07K 16/28; A61P 35/02; A61K 9/00

(52) U.S. Cl. CPC A61K 35/28; A61K 47/6807; A61K 45/06; C12N 5/0647; C07K 16/2803; A61P 35/02; A61K 9/0019

(57) ABSTRACT

Aspects of the disclosure provide methods and compositions for treating a hematopoietic malignancy (e.g., acute myeloid leukemia). In some aspects, the disclosure provides methods of treatment using a population of genetically engineered CD33-deficient hematopoietic cells and a cytotoxic agent comprising an anti-CD33 antigen-binding domain.

### RELATED APPLICATIONS

The application claims the benefit under 35 U.S.C. 119(e) of U.S. provisional application No. 63/106,136, filed Oct. 27, 2020, and U.S. provisional application No. 63/165,950, filed Mar. 25, 2021, which are incorporated by reference herein in their entireties.

### BACKGROUND

Hematopoietic cell transplantation (HCT) can be used to treat patients with acute myeloid leukemia (AML) in complete remission but at high risk of relapse, especially those with intermediate or adverse disease-related genetics. With the reduction in transplant-related mortality over the past several decades, leukemia relapse post-HCT remains an obstacle to improved overall outcomes. Several recent studies have revealed that the presence of minimal residual disease (MRD) while in morphological complete remission at the time of HCT is independently associated with a significantly increased risk of leukemia relapse and death. In addition, patients who present with evidence of persistent leukemic blasts in the bone marrow are individuals at very high risk for early relapse post HCT.

### SUMMARY

The methods described herein provide new approaches to reduce the risk of leukemia relapse and improve overall outcomes in patients with acute myeloid leukemia. In some aspects, the present disclosure provides a method comprising administering to a subject an effective amount of a population of genetically engineered hematopoietic cells, or descendants thereof, comprising a modified gene encoding CD33 that is engineered to have reduced or eliminated expression of a CD33 antigen; and an effective amount of a cytotoxic agent comprising an anti-CD33 antigen-binding domain.

In some aspects, the present disclosure provides a method comprising administering to a subject an effective amount of a cytotoxic agent comprising an anti-CD33 antigen-binding domain. In some embodiments, the subject is receiving or has received an effective amount of a population of genetically engineered hematopoietic cells, or descendants thereof, comprising a modified gene encoding CD33 that is engineered to have reduced or eliminated expression of a CD33 antigen.

In some aspects, the present disclosure provides a method comprising administering to a subject an effective amount of a population of genetically engineered hematopoietic cells, or descendants thereof, comprising a modified gene encoding CD33 that is engineered to have reduced or eliminated expression of a CD33 agent. In some embodiments, the subject is receiving or has received an effective amount of a cytotoxic agent comprising an anti-CD33 antigen-binding domain.

In some embodiments, the cytotoxic agent is an antibody-drug conjugate (ADC). In some embodiments, the ADC is gemtuzumab ozogamicin (GO).

In some embodiments, the effective amount of the population of genetically engineered hematopoietic cells is about 10⁶ cells/kilogram body weight of the subject to about 10⁷ cells/kilogram body weight of the subject. In some embodiments, the effective amount of the population of genetically engineered hematopoietic cells is about 3.0×10⁶ cells/kilogram body weight of the subject.

In some embodiments, the effective amount of the cytotoxic agent is about 0.1 mg/m² body surface area of the subject to about 2.0 mg/m² body surface area of the subject. In some embodiments, the effective amount of the cytotoxic agent is about 0.1 mg/m², about 0.25 mg/m², about 0.5 mg/m², about 1.0 mg/m², or about 2.0 mg/m² body surface area of the subject. In some embodiments, the effective amount of the cytotoxic agent is about 2.0 mg/m² body surface area of the subject.

In some embodiments, the population of genetically engineered hematopoietic cells and the cytotoxic agent are administered in temporal proximity. In some embodiments, administering in temporal proximity comprises administering the population of genetically engineered hematopoietic cells and the cytotoxic agent within a single treatment regimen. In some embodiments, administering in temporal proximity comprises administering the population of genetically engineered hematopoietic cells and the cytotoxic agent simultaneously. In some embodiments, administering in temporal proximity comprises administering the population of genetically engineered hematopoietic cells and the cytotoxic agent concurrently. In some embodiments, administering in temporal proximity comprises administering the population of genetically engineered hematopoietic cells and the cytotoxic agent sequentially. In some embodiments, administering in temporal proximity comprises administering the population of genetically engineered hematopoietic cells within 120 days of administering the cytotoxic agent (e.g., within 90 days of administering the cytotoxic agent, within 60 days of administering the cytotoxic agent).

In some embodiments, the population of genetically engineered hematopoietic cells are administered prior to the cytotoxic agent. In some embodiments, the population of genetically engineered hematopoietic cells are administered in a single treatment regimen. In some embodiments, the population of genetically engineered hematopoietic cells are administered intravenously.

In some embodiments, the cytotoxic agent is administered in multiple doses of the effective amount every four weeks. In some embodiments, the cytotoxic agent is administered in multiple doses of about 2.0 mg/m² every four weeks.

In some embodiments, the population of genetically engineered hematopoietic cells are thawed from a cryopreserved form prior to administration. In some embodiments, the cytotoxic agent is reconstituted from a lyophilized form prior to administration.

In some embodiments, the subject has been preconditioned prior to administering the cytotoxic agent and/or the population of genetically engineered hematopoietic cells. In some embodiments, the method further comprises preconditioning the subject prior to administering the cytotoxic agent and/or the population of genetically engineered hematopoietic cells. In some embodiments, the preconditioning comprises administering one or more chemotherapeutic agents to the subject. In some embodiments, the preconditioning comprises total body irradiation of the subject. In some embodiments, the chemotherapeutic agent is selected from the group consisting of busulfan, melphalan, fludarabine, cyclophosphamide, and thiotepa. In some embodiments, the preconditioning comprises administering antibodies that bind human T cells (T lymphocytes), optionally wherein the antibodies comprise rabbit anti-thymocyte globulins (rATG).

In some embodiments, the subject has, or has been diagnosed with, a hematopoietic malignancy or a hematopoietic pre-malignant disease, and wherein the hematopoietic malignancy is characterized by the presence of CD33-positive malignant cells, or wherein the hematopoietic pre-malignant disease is characterized by the presence of CD33-positive pre-malignant cells. In some embodiments, the subject has, or has been diagnosed with, CD33-positive acute myeloid leukemia. In some embodiments, the subject has, or has been diagnosed with, CD33-positive myelodysplastic syndrome. In some embodiments, the subject has, or has been diagnosed with, CD33-positive myelodysplastic syndrome and wherein the subject is at high risk of developing acute myeloid leukemia.

In some embodiments, the subject is naïve to chemotherapy and/or radiation therapy. In some embodiments, the subject is naïve to any treatment aimed to address a hematopoietic malignancy or hematopoietic pre-malignant disease.

In some embodiments, subject has previously received chemotherapy. In some embodiments, the subject has previously received induction therapy. In some embodiments, the subject has previously entered a complete hematological remission, optionally wherein the complete hematological remission is characterized by an incomplete recovery of peripheral counts. In some embodiments, the subject has one or more risk factors associated with early leukemia relapse. In some embodiments, the one or more risk factors associated with early leukemia relapse are selected from the group consisting of bone marrow in morphological complete remission with presence of intermediate or high-risk disease-related genetics; presence of minimal residual disease (MRD) post cyto-reductive therapy; bone marrow with persistent leukemia blasts post cyto-reductive therapy; and bone marrow blast count of about 10% or less with no circulating blasts.

In some embodiments, the subject does not have a homozygous dominant genotype for CD33 single nucleotide polymorphism (SNP) rs12459419. In some embodiments, the subject does not have acute promyelocytic leukemia or chronic myeloid leukemia. In some embodiments, the subject does not have a genetic translocation associated with acute promyelocytic leukemia or chronic myeloid leukemia, optionally wherein the genetic translocation is t(15; 17)(q22; q21) or t(9; 22)(q34; q11). In some embodiments, the subject has not previously received an autologous or allogeneic stem cell transplantation. In some embodiments, the subject has not previously received the cytotoxic agent.

In some embodiments, the method further comprises determining a percent donor chimerism and/or a level of CD33-negative myeloid hematopoiesis in a peripheral blood sample from the subject. In some embodiments, the subject has a CD33-negative absolute neutrophil count (ANC) of at least 1000/dL prior to receiving the cytotoxic agent.

In some embodiments, the population of genetically engineered hematopoietic cells are hematopoietic stem cells. In some embodiments, the hematopoietic stem cells are from bone marrow cells, cord blood cells, or peripheral blood mononuclear cells (PBMCs). In some embodiments, the hematopoietic stem cells are CD34⁺/CD33⁻. In some embodiments, the hematopoietic cells are autologous. In some embodiments, the method further comprises obtaining the autologous hematopoietic stem cells from the subject, optionally wherein the method further comprises genetically engineering the autologous stem cells to have reduced or eliminated expression of the CD33 antigen, and returning the genetically engineered hematopoietic stem cells to the subject.

In some embodiments, the hematopoietic cells are allogeneic. In some embodiments, the hematopoietic cells are allogeneic hematopoietic stem cells obtained from a donor having an HLA haplotype that matches with the HLA haplotype of the subject. In some embodiments, the method further comprises obtaining hematopoietic cells from a donor having an HLA haplotype that matches with the HLA haplotype of the subject.

In some embodiments, the method further comprises preparing the population of genetically engineered hematopoietic cells by modifying an endogenous gene of the hematopoietic cells encoding the CD33 antigen. In some embodiments, the whole or a portion of the endogenous gene encoding the CD33 cell-surface antigen is deleted. In some embodiments, the whole or the portion of the endogenous gene is deleted using genome editing. In some embodiments, the genome editing involves a zinc finger nuclease (ZFN), a transcription activator-like effector-based nuclease (TALEN), or a CRISPR-Cas system.

In some aspects, the present disclosure provides compositions comprising a population of genetically modified hematopoietic cells, or descendants thereof, comprising a modified gene encoding CD33 that is engineered to have reduced or eliminated expression of a CD33 antigen. In some embodiments, the hematopoietic cells are hematopoietic stem cells from bone marrow cells, cord blood cells, or peripheral blood mononuclear cells (PBMCs). In some embodiments, the hematopoietic stem cells are CD34+/CD33−.

In some embodiments, the whole or a portion of an endogenous gene encoding the CD33 antigen is deleted. In some embodiments, the whole or the portion of the endogenous gene is deleted using genome editing. In some embodiments, the genome editing carried out involves a zinc finger nuclease (ZFN), a transcription activator-like effector-based nuclease (TALEN), or a CRISPR-Cas system. In some embodiments, the CRISPR-Cas system comprises a nucleic acid encoding a gRNA and an RNA-guided nuclease. In some embodiments, the gRNA comprises a targeting domain comprising a sequence of any one of SEQ ID NOs: 9-15.

In some aspects, the present disclosure provides combinations comprising a population of any of the genetically modified hematopoietic cells described herein and a cytotoxic agent comprising an anti-CD33 antigen-binding domain. In some embodiments, the cytotoxic agent is an antibody-drug conjugate (ADC). In some embodiments, the ADC is gemtuzumab ozogamicin.

In some aspects, the present disclosure provides compositions comprising a population of at least 1×10⁶ cells per milliliter (mL) in a medium, wherein the population of cells comprise genetically modified hematopoietic cells, or descendants thereof, comprising a modified gene encoding CD33 that is engineered to have reduced or eliminated expression of a CD33 antigen. In some embodiments, the medium has a volume between about 40-50 mL. In some embodiments, the medium has a volume between about 5-150 mL. In some embodiments, the medium has a volume between about 10-100 mL. In some embodiments, the medium has a volume between about 25-75 mL. In some embodiments, the medium has a volume between about 30-70 mL. In some embodiments, the medium has a volume between about 40-60 mL. In some embodiments, the medium has a volume of about 45 mL. In some embodiments, the medium has a volume of about 30 mL. In some embodiments, the medium has a volume of about 35 mL. In some embodiments, the medium has a volume of about 40 mL. In some embodiments, the medium has a volume of about 50 mL. In some embodiments, the medium has a volume of about 55 mL. In some embodiments, the medium has a volume of about 60 mL. In some embodiments, the medium has a volume of about 70 mL.

In some embodiments, at least 50%, at least 60%, at least 70%, at least 80%, at least 90%, at least 95%, at least 97%, at least 98%, or at least 99% of the cells of the population are genetically modified hematopoietic cells, or descendants thereof, having reduced or eliminated expression of a CD33 antigen. In some embodiments, the population comprises at least 0.5×10⁶ cells per mL, at least 1×10⁶ cells per mL, at least 2×10⁶ cells per mL, at least 3×10⁶ cells per mL, at least 4×10⁶ cells per mL, at least 5×10⁶ cells per mL, at least 6×6 cells per mL, at least at least 7×10⁶ cells per mL, at least 8×10⁶ cells per mL, or at least 9×10⁶ cells per mL. In some embodiments, the population comprises at least 0.5×10⁶ cells per mL, at least 1×10⁶ cells per mL, at least 2×10⁶ cells per mL, at least 3×10⁶ cells per mL, at least 4×10⁶ cells per mL, at least 5×10⁶ cells per mL, at least 6×10⁶ cells per mL, at least at least 7×10⁶ cells per mL, at least 8×10⁶ cells per mL, or at least 9×10⁶ cells per mL. In some embodiments, the cell population comprises at least 1×10⁹ viable cells, at least 2×10⁹ viable cells, at least 3×10⁹ viable cells, at least 4×10⁹ viable cells, at least 5×10⁹ viable cells, at least 6×10⁹ viable cells, at least 7×10⁹ viable cells, at least 8×10⁹ viable cells, at least 9×10⁹ viable cells, at least 1×10¹⁰ viable cells, at least 2×10¹⁰ viable cells, at least 3×10¹⁰ viable cells, at least 4×10¹⁰ viable cells, at least 5×10¹⁰ viable cells, at least 6×10¹⁰ viable cells, at least 7×10¹⁰ viable cells, at least 8×10¹⁰ viable cells, at least 9×10¹⁰ viable cells, or at least 1×10¹¹ viable cells, wherein, in some embodiments, at least 50%, at least 60%, at least 70%, at least 80%, at least 90%, at least 95%, at least 97%, at least 98%, or at least 99% of the cells of the population are genetically modified hematopoietic cells, or descendants thereof, having reduced or eliminated expression of a CD33 antigen.

In some embodiments, the medium is a cryopreservation medium comprising a cryoprotectant. In some embodiments, the cryoprotectant comprises dimethylsulfoxide (DMSO) in an amount of about 10%. (v/v).

In some embodiments, the hematopoietic cells are CD34+/CD33−. In some embodiments, the whole or a portion of an endogenous gene encoding the CD33 antigen is deleted. In some embodiments, the whole or the portion of the endogenous gene is deleted using genome editing. In some embodiments, the genome editing carried out involves a zinc finger nuclease (ZFN), a transcription activator-like effector-based nuclease (TALEN), or a CRISPR-Cas system. In some embodiments, the CRISPR-Cas system comprises a nucleic acid encoding a gRNA and an RNA-guided nuclease. In some embodiments, the gRNA comprises a targeting domain comprising a sequence of any one of SEQ ID NOs: 9-15. In some embodiments, the compositions does not comprise a detectable level of the RNA-guided nuclease.

In some embodiments, the composition is in a frozen state.

In some embodiments, the composition has been subjected to a cryopreservation process. In some embodiments, the cryopreservation process is controlled-rate freezing.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings, which constitute a part of this specification, illustrate several embodiments of the invention and together with the description, serve to explain the principles of the invention.

FIG. 1 is a schematic showing an example of a dosing regimen for subjects in a clinical study.

FIG. 2 is a schematic showing an example experimental design to evaluate efficacy of the methods described herein. Briefly, CD34+ hematopoietic stem and progenitor cells (HSPCs) from mobilized PMBCs (mPBMC) obtained from a human donor. The HSPCs are either mock electroporated (“control”) or subjected to genome editing to reduce or eliminate the expression of a CD33 antigen (CD33KO), for example using CD33 gRNAs and a CRISPR-Cas system, and then administered to sub-lethally irradiated NOD/scid/IL2Rγ^{null} ((NOD.Cg-Prkdc^{scid}Il2rg^{tm1Wjl}, NSG™) mice. Blood samples are obtained at 8-week and 12-week post HSPC transplantation time points. The mice are administered a cytotoxic agent comprising an anti-CD33 antigen-binding domain, such as gemtuzumab ozogamicin/Mylotarg® at a dose of 0.33 mg/kg, or vehicle control, at approximately 15-weeks. Approximately 8 days following gemtuzumab ozogamicin/Mylotarg® treatment (16 weeks post HSPC transplantation), mice are sacrificed and samples of terminal bone marrow (BM), blood, and spleen are harvested and subjected to analysis, for example by flow cytometry.

FIGS. 3A-3E present plots showing results of flow cytometric analysis of cell populations following transplantation of human HSPCs from Donor 1, according to the experimental design shown in FIG. 2. FIG. 3A shows human leukocyte chimerism as determined by the percent human CD45+ cells. FIG. 3B shows the CD33+ myeloid cells as the percent of human CD45+ cells. FIG. 3C shows the CD14+ myeloid cells as the percent human CD45+ cells. FIG. 3D shows the CD11b+ myeloid cells as the percent of human CD45+ cells. FIG. 3E shows the presence of CD33 in CD14+ myeloid cells, indicating the CD14+ cells that were edited for CD33 were protected from gemtuzumab ozogamicin/Mylotarg® cytotoxicity. For each plot, Mock EP refers to transplantation with human HSPCs that were mock electroporated, and CD33 gRNA refers to transplantation with human HSPCs that were edited using a CD33 gRNA. “Mylotarg” refers to mice that received gemtuzumab ozogamicin/Mylotarg® treatment, and “vehicle” refers to control mice that received the vehicle control. Each data point represents individual values for a mouse, with the mean and standard deviation indicated (n=9-10). *=P<0.05, **=P<0.01, ***=P<****=P<0.0001.

FIGS. 4A-4C present plots showing results of flow cytometric analysis of cell populations following transplantation of human HSPCs from Donor 1, according to the experimental design shown in FIG. 2. FIG. 4A shows CD3+ T cells as the percent human CD45+ cells. FIG. 4B shows the CD19+ B cells as the percent of human CD45+ cells. FIG. 4C shows the CD34+CD38− primitive HSPCs as the percent human CD45+ cells. For each plot, Mock EP refers to transplantation with human HSPCs that were mock electroporated, and CD33 gRNA refers to transplantation with human HSPCs that were edited using a CD33 gRNA. “Mylotarg” refers to mice that received gemtuzumab ozogamicin/Mylotarg® treatment, and “vehicle” refers to control mice that received the vehicle control. Each data point represents individual values for a mouse, with the mean and standard deviation indicated (n=9-10). *=P<0.05, **=P<0.01, ***=P<0.001, ****=P<0.0001.

FIGS. 5A-5D present plots showing results of flow cytometric analysis of cell populations following transplantation of human HSPCs from Donor 2, according to the experimental design shown in FIG. 2. FIG. 5A shows human leukocyte chimerism as determined by the percent human CD45+ cells. FIG. 5B shows the CD33+ myeloid cells as the percent of human CD45+ cells. FIG. 5C shows the CD14+ myeloid cells as the percent human CD45+ cells. FIG. 5D shows the CD11b+ myeloid cells as the percent of human CD45+ cells. For each plot, Mock EP refers to transplantation with human HSPCs that were mock electroporated, and CD33 gRNA refers to transplantation with human HSPCs that were edited using a CD33 gRNA. “Mylotarg” refers to mice that received gemtuzumab ozogamicin/Mylotarg® treatment, and “vehicle” refers to control mice that received the vehicle control. Each data point represents individual values for a mouse, with the mean and standard deviation indicated (n=9-10). *=P<0.05, **=P<0.01, ***=P<0.001, ****=P<0.0001.

FIGS. 6A-6C present plots showing results of flow cytometric analysis of cell populations following transplantation of human HSPCs from Donor 2, according to the experimental design shown in FIG. 2. FIG. 6A shows CD3+ T cells as the percent human CD45+ cells. FIG. 6B shows the CD19+ B cells as the percent of human CD45+ cells. FIG. 6C shows the CD34+CD38− primitive HSPCs as the percent human CD45+ cells. For each plot, Mock EP refers to transplantation with human HSPCs that were mock electroporated, and CD33 gRNA refers to transplantation with human HSPCs that were edited using a CD33 gRNA. “Mylotarg” refers to mice that received gemtuzumab ozogamicin/Mylotarg® treatment, and “vehicle” refers to control mice that received the vehicle control. Each data point represents individual values for a mouse, with the mean and standard deviation indicated (n=9-10). *=P<0.05, **=P<0.01, ***=P<0.001, ****=P<0.0001.

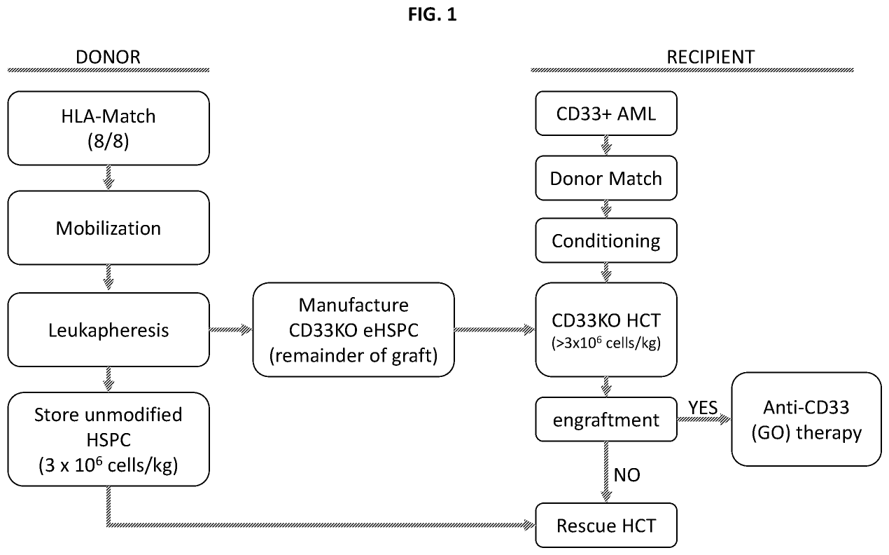

### DETAILED DESCRIPTION

The present disclosure provides targeted therapeutic approaches for use in treating hematopoietic malignancies that overcome limitations in existing therapies. For example, current CD33-targeted therapies for acute myeloid leukemia (AML) are limited by “on-target, off-leukemia” cytotoxicity directed toward normal healthy myeloid lineage cells expressing CD33. The loss of the noncancerous CD33+ cells can deplete the hematopoietic system of the patient. To address this depletion, the subject can be administered rescue cells (e.g., hematopoietic cells) comprising a modification in the CD33 gene. These CD33-modified cells can be resistant to the anti-CD33 cancer therapy and can therefore repopulate the hematopoietic system during or after anti-CD33 therapy. In this way, the normal myeloid compartment is protected from the on-target effects of CD33-targeted agents, resulting in an improved therapeutic index for these agents and better patient outcomes.

### Cells

Aspects of the present disclosure related to genetically engineered hematopoietic cells (also referred to herein as eHSCs or eHSPCs), or descendants thereof, comprising a modified gene encoding CD33 that is engineered to have reduced or eliminated expression of a CD33 antigen. In some embodiments, genetically engineered hematopoietic cells of the disclosure (e.g., a hematopoietic stem cells (HSC) or hematopoietic progenitor cells (HPC)) having a modification of the gene encoding CD33 are genetically engineered using any genetic editing methods known in the art.

In some embodiments, genetically engineered hematopoietic cells of the disclosure (e.g., a hematopoietic stem cells (HSC) or hematopoietic progenitor cells (HPC)) having a modification of the gene encoding CD33 are genetically engineered using a nuclease and/or a gRNA described herein. In some embodiments, a cell (e.g., HSC or HPC) having a modification of CD33 and a modification of a second lineage-specific cell surface antigen is made using a nuclease and/or a gRNA described herein. It is understood that the cell can be made by contacting the cell itself with the nuclease and/or a gRNA, or the cell can be the daughter cell of a cell that was contacted with the nuclease and/or gRNA. In some embodiments, a cell described herein (e.g., an HSC or HPC) is capable of reconstituting the hematopoietic system of a subject. In some embodiments, a cell described herein (e.g., an HSC or HPC) is capable of one or more of (e.g., all of): engrafting in a human subject, producing myeloid lineage cells, and producing and lymphoid lineage cells.

In some embodiments, the cell comprises only one genetic modification. In some embodiments, the cell is only genetically modified at the CD33 locus, such as in a sequence of exon 3 of CD33. In some embodiments, the cell is genetically modified at a second locus. In some embodiments, the cell does not comprise a transgenic protein, e.g., does not comprise a chimeric antigen receptor (CAR).

The terms “CD33 antigen” and “CD33 protein” are used interchangeably herein and refer to the CD33 protein, or a portion or fragment thereof, such as a portion that is targeted by an anti-CD33 agent, such as a cytotoxic agent comprising an anti-CD33 antigen-binding domain.

In some embodiments, a genetically engineered hematopoietic cell described herein comprises substantially no CD33 protein (CD33 antigen). In some embodiments, a genetically engineered hematopoietic cell described herein comprises substantially no wild-type CD33 protein but comprises a mutant CD33 protein. In some embodiments, the mutant CD33 protein is not bound by an agent that targets CD33 for therapeutic purposes. As used herein, a genetically engineered hematopoietic cell that has been genetically engineered such that the cell has reduced or no expression of CD33 may be referred to as “CD33KO eHSCs” or “CD33KO eHSPCs.”

In some embodiments, the cell is a circulating blood cell, e.g., a reticulocyte, megakaryocyte erythroid progenitor (MEP) cell, myeloid progenitor cell (CMP/GMP), lymphoid progenitor (LP) cell, hematopoietic stem cell (HSC) or hematopoietic progenitor cell (HPC), which may be referred to as hematopoietic stem and progenitor cells (HSPCs), or endothelial cell (EC). In some embodiments, the cell is a bone marrow cell (e.g., a reticulocyte, an erythroid cell (e.g., erythroblast), megakaryocyte-erythroid progenitor cell (MEP cell), myeloid progenitor cell (CMP/GMP), lymphocyte predominant (LP) cell, erythroid progenitor (EP) cell, HSC, multipotent progenitor (MPP) cell, endothelial cell (EC), hemogenic endothelial (HE) cell, or mesenchymal stem cell). In some embodiments, the cell is a myeloid progenitor cell (e.g., a common myeloid progenitor (CMP) cell or granulocyte macrophage progenitor (GMP) cell). In some embodiments, the cell is a lymphoid progenitor cell, e.g., a common lymphoid progenitor (CLP) cell. In some embodiments, the cell is an erythroid progenitor cell (e.g., an MEP cell). In some embodiments, the cell is a hematopoietic stem/progenitor cell (e.g., a long-term HSC (LT-HSC), short term HSC (ST-HSC), MPP cell, or lineage restricted progenitor (LRP) cell). In some embodiments, the cell is a CD34+ cell, CD34+CD90+ cell, CD34+CD38+ cell, CD34+CD90+CD49\hat{ }CD38+CD45RA cell, CD105+ cell, CD31+, or CD133+ cell, or a CD34+CD90+CD133+ cell. In some embodiments, the cell is an umbilical cord blood CD34+ HSPC, umbilical cord venous endothelial cell, umbilical cord arterial endothelial cell, amniotic fluid CD34+ cell, amniotic fluid endothelial cell, placental endothelial cell, or placental hematopoietic CD34+ cell. In some embodiments, the cell is a mobilized peripheral blood hematopoietic CD34+ cell (after the patient is treated with a mobilization agent, e.g., G-CSF and/or Plerixafor). In some embodiments, the cell is a peripheral blood endothelial cell, or population of cells.

In some embodiments, the cells are hematopoietic cells, e.g., hematopoietic stem cells. Hematopoietic stem cells (HSCs) are typically capable of giving rise to both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. HSCs are characterized by the expression of the cell surface marker CD34 (e.g., CD34+), which can be used for the identification and/or isolation of HSCs, and absence of cell surface markers associated with commitment to a cell lineage.

In some embodiments, a population of genetically engineered hematopoietic cells described herein comprises a plurality of hematopoietic stem cells. In some embodiments, a population of genetically engineered hematopoietic cells described herein comprises a plurality of hematopoietic progenitor cells. In some embodiments, a population of genetically engineered hematopoietic cells described herein comprises a plurality of hematopoietic stem cells and a plurality of hematopoietic progenitor cells.

In some embodiments, a hematopoietic stem cell (HSC) refers to cells of a stem cell lineage that give rise to all the blood cell types including the erythroid (erythrocytes or red blood cells (RBCs)), myeloid (monocytes and macrophages, neutrophils, basophils, eosinophils, megakaryocytes/platelets, and dendritic cells), and lymphoid (T-cells, B-cells, NK-cells). In some embodiments, the cells used herein are selected from the group consisting of a circulating blood cell, a mobilized blood cell, a bone marrow cell, a myeloid progenitor cell, a lymphoid progenitor cell, a multipotent progenitor cell, a lineage restricted progenitor cell, an endothelial cell, or a mesenchymal stromal cell. In some embodiments, the HSC is from a non-cord blood source, an umbilical cord source, or a cord blood source. In one embodiment, the HSC is a CD34+ cell. In some embodiments, the HSC cell is capable of differentiating in vivo after transplantation into the subject. In some embodiments, the HSC cell is capable of differentiating into B cells, T cells, erythroid cells, and/or myeloid cells. In some embodiments, the HSC cell is capable of reconstituting hematopoiesis in the subject. In some embodiments, the hematopoietic stem cell has at least one of the cell surface marker characteristic of hematopoietic progenitor cells: CD34+, CD59+, Thyl/CD90+, CD38lo/−, and C-kit/CD117+. In some embodiments, the hematopoietic progenitor are CD34+.

In some embodiments, a hematopoietic stem cell is a peripheral blood stem cell obtained from a subject after the subject has been treated with granulocyte colony stimulating factor (G-CSF) (optionally in combination with plerixafor). In some embodiments, CD34+ cells are enriched using CliniMACS® Cell Selection System (Miltenyi Biotec). In some embodiments, CD34+ cells are weakly stimulated in serum-free medium (e.g., CellGrow SCGM media, CellGenix) with cytokines (e.g., SCF, rhTPO, rhFLT3) before genome editing. In some embodiments, addition of SR1 and dmPGE2 and/or other factors is contemplated to improve long-term engraftment.

In some embodiments, a population of genetically engineered hematopoietic cells for administration in accordance with the disclosure can be allogeneic hematopoietic progenitor cells obtained from one or more donors. As used herein, “allogeneic” refers to a hematopoietic progenitor cell or biological samples comprising hematopoietic progenitor cells obtained from one or more different donors of the same species, where the genes at one or more loci are not identical. For example, a hematopoietic cell population being administered to a subject can be derived from umbilical cord blood obtained from one more unrelated donor subjects, or from one or more non-identical siblings. In some embodiments, syngeneic hematopoietic cell populations can be used, such as those obtained from genetically identical animals, or from identical twins. In some embodiments, the hematopoietic cells are autologous cells; that is, the hematopoietic progenitor cells are obtained or isolated from a subject and administered to the same subject (i.e., the donor and recipient are the same).

In some embodiments, a population of genetically engineered hematopoietic cells described herein comprises a plurality of genetically engineered hematopoietic stem cells. In some embodiments, a population of genetically engineered hematopoietic cells described herein comprises a plurality of genetically engineered hematopoietic progenitor cells. In some embodiments, population of genetically engineered hematopoietic cells described herein comprises a plurality of genetically engineered hematopoietic stem cells and a plurality of genetically engineered hematopoietic progenitor cells

In some embodiments, the HSCs or HPCs are obtained from a subject, such as a human subject. Methods of obtaining HSCs are described, e.g., in PCT Application No. US2016/057339, which is herein incorporated by reference in its entirety. In some embodiments, the HSCs are peripheral blood HSCs. In some embodiments, the mammalian subject is a non-human primate, a rodent (e.g., mouse or rat), a bovine, a porcine, an equine, or a domestic animal. In some embodiments, the HSCs are obtained from a human subject, such as a human subject having a hematopoietic malignancy. In some embodiments, the HSCs or HPCs are obtained from a healthy donor. In some embodiments, the HSCs or HPCs are obtained from the subject to whom the cytotoxic agent comprising an anti-CD33 antigen-binding domain will be subsequently administered. HSCs or HPCs that are administered to the same subject from which the cells were obtained are referred to as autologous cells, whereas HSCs or HPCs that are obtained from a subject who is not the subject to whom the cells will be administered are referred to as allogeneic cells.

In some embodiments, a population of genetically engineered hematopoietic cells is a heterogeneous population of cells, e.g. heterogeneous population of genetically engineered hematopoietic cells containing different CD33 mutations. In some embodiments, at least 40%, at least 50%, at least 60%, at least 70%, at least 75%, at least 80%, at least 85%, at least 90%, or at least 95% of copies of CD33 in the population of cells have a mutation effected by a genome editing approach described herein, e.g. by a CRISPR/Cas system using a gRNA described herein. By way of example, a population can comprise a plurality of different CD33 mutations and each mutation of the plurality contributes to the percent of copies of CD33 in the population of cells that have a mutation.

In some embodiments, the expression of CD33 on the genetically engineered hematopoietic cell is compared to the expression of CD33 on a naturally occurring hematopoietic cell (e.g., a wild-type counterpart). In some embodiments, the genetic engineering results in a reduction in the expression level of CD33 by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of CD33 on a naturally occurring hematopoietic cell (e.g., a wild-type counterpart). For example, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of CD33 as compared to a naturally occurring hematopoietic cell (e.g., a wild-type counterpart).

In some embodiments, the genetic engineering results in a reduction in the expression level of wild-type CD33 by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of the level of wild-type CD33 on a naturally occurring hematopoietic cell (e.g., a wild-type counterpart). For example, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of CD33 as compared to a naturally occurring hematopoietic cell (e.g., a wild-type counterpart).

In some embodiments, the genetic engineering results in a reduction in the expression level of wild-type lineage-specific cell surface antigen (e.g., CD33) by at least 50%, at least 60%, at least 70%, at least 80%, at least 90%, at least 91%, at least 92%, at least 93%, at least 94%, at least 95%, at least 96%, at least 97%, at least 98%, or at least 99% as compared to a suitable control (e.g., a cell or plurality of cells). In some embodiments, the suitable control comprises the level of the wild-type lineage-specific cell surface antigen measured or expected in a plurality of non-engineered cells from the same subject. In some embodiments, the suitable control comprises the level of the wild-type lineage-specific cell surface antigen measured or expected in a plurality of cells from a healthy subject. In some embodiments, the suitable control comprises the level of the wild-type lineage-specific cell surface antigen measured or expected in a population of cells from a pool of healthy individuals (e.g., 10, 20, or 100 individuals). In some embodiments, the suitable control comprises the level of the wild-type lineage-specific cell surface antigen measured or expected in a subject in need of a treatment described herein, e.g., an anti-CD33 therapy, e.g., wherein the subject has a cancer, wherein cells of the cancer express CD33. In some embodiments, the suitable control comprises the level of the wild-type lineage-specific cell surface antigen measured in the cells prior to being subjected to genetic engineering to reduce or eliminate expression of CD33.

In some embodiments, a method of making the genetically engineered hematopoietic cells described herein comprises a step of providing a wild-type cell, e.g., a wild-type hematopoietic stem or progenitor cell. In some embodiments, the wild-type cell is an unedited cell comprising (e.g., expressing) two functional copies of the lineage-specific cell surface antigen (e.g., CD33). In some embodiments, the cell comprises a CD33 gene sequence according to SEQ ID NO: 16. In some embodiments, the cell comprises a CD33 gene sequence encoding a CD33 protein that is encoded in SEQ ID NO: 16, e.g., the CD33 gene sequence may comprise one or more silent mutations relative to SEQ ID NO: 16. In some embodiments, the wild-type cell expresses the lineage-specific cell surface antigen (e.g., CD33), or gives rise to a more differentiated cell that expresses the lineage-specific cell surface antigen at a level comparable to (or within 90%-110%, 80%-120%, 70%-130%, 60-140%, or 50%-150% of) HL60 or MOLM-13 cells. In some embodiments, the wild-type cell binds an antibody that binds the lineage-specific cell surface antigen (e.g., an anti-CD33 antibody, e.g., P67.6), or gives rise to a more differentiated cell that binds the antibody at a level comparable to (or within 90%-110%, 80%-120%, 70%-130%, 60-140%, or 50%-150% of) binding of the antibody to HL60 or MOLM-13 cells. Antibody binding may be measured, for example, by flow cytometry.

In some aspects, the genetically engineered hematopoietic stem or progenitor cell comprises a genetic mutation in the exon 3 of an endogenous CD33 gene, wherein the genetic mutation is at a site described herein (see, Table 1). One aspect of the present disclosure provides a genetically engineered hematopoietic stem and/or progenitor cell comprises a genetic mutation in exon 3 of an endogenous CD33 gene, wherein the genetic mutation is at a site targeted by a gRNA, such as any of the gRNAs presented in Table 1.

In some embodiments, an engineered cell described herein comprises two mutations, the first mutation being in CD33 and the second mutation being in a second lineage-specific cell surface antigen. Such a cell can, in some embodiments, be resistant to two agents: an anti-CD33 agent and an agent targeting the second lineage-specific cell surface antigen. In some embodiments, such a cell can be produced using two or more gRNAs described herein, e.g., a gRNA of Table 3 and a second gRNA. In some embodiments, the cell can be produced using, e.g., a ZFN or TALEN. The disclosure also provides populations comprising cells described herein.

In some embodiments, the second mutation is at a gene encoding a lineage-specific cell-surface antigen, such as any of the lineage-specific cell-surface antigens described herein.

Typically, a mutation effected by the methods and compositions provided herein, e.g., a mutation in a target gene, such as, for example, CD33 results in a loss of function of a gene product encoded by the target gene, e.g., in the case of a mutation in the CD33 gene, in a loss of function of a CD33 protein. In some embodiments, the loss of function is a reduction in the level of expression of the gene product, e.g., reduction to a lower level of expression, or a complete abolishment of expression of the gene product. In some embodiments, the mutation results in the expression of a non-functional variant of the gene product. For example, in the case of the mutation generating a premature stop codon in the encoding sequence, a truncated gene product, or, in the case of the mutation generating a nonsense or missense mutation, a gene product characterized by an altered amino acid sequence, which renders the gene product non-functional. In some embodiments, the function of a gene product is binding or recognition of a binding partner. In some embodiments, the reduction in expression of the gene product, e.g., of CD33, of the second lineage-specific cell-surface antigen, or both, is to less than or equal to 50%, less than or equal to 40%, less than or equal to 30%, less than or equal to 20%, less than or equal to 10%, less than or equal to 5%, less than or equal to 2%, or less than or equal to 1% of the level in a wild-type or non-engineered counterpart cell.

In some embodiments, at least 40%, at least 50%, at least 60%, at least 70%, at least 75%, at least 80%, at least 85%, at least 90%, or at least 95%, or more of copies of CD33 in the population of genetically engineered hematopoietic cells generated by the methods and/or using the compositions provided herein have a mutation. In some embodiments, at least 40%, at least 50%, at least 60%, at least 70%, at least 75%, at least 80%, at least 85%, at least 90%, or at least 95% of copies of the second lineage-specific cell surface antigen in the population of genetically engineered hematopoietic cells have a mutation. In some embodiments, at least 40%, at least 50%, at least 60%, at least 70%, at least 75%, at least 80%, at least 85%, at least 90%, or at least 95% of copies of CD33 and of the second lineage-specific cell surface antigen in the population of genetically engineered hematopoietic cells have a mutation. In some embodiments, the population comprises one or more wild-type cells. In some embodiments, the population comprises one or more cells that comprise one wild-type copy of CD33. In some embodiments, the population comprises one or more cells that comprise one wild-type copy of the second lineage-specific cell surface antigen.

### Cytotoxic Agents

Aspects of the present disclosure related to cytotoxic agents comprising an anti-CD33 antigen-binding domain. Administration of a cytotoxic agent comprising an anti-CD33 antigen-binding domain interacts with and induced cytotoxicity of cells expressing CD33. As described herein, administration of such cytotoxic agents may induce cytotoxicity of not only cancer cells expressing CD33 but also normal, healthy cells that also express CD33, e.g., “on-target, off-leukemia” effects.

In some embodiments, a cytotoxic agent of the present disclosure is an antibody-drug conjugate (ADC). The ADC may be a molecule comprising an antibody or antigen-binding fragment thereof conjugated to a toxin or drug molecule. Binding of the antibody or fragment thereof to the corresponding antigen allows for delivery of the toxin or drug molecule to a cell that presents the antigen on its cell surface (e.g., target cell), thereby resulting in death of the target cell.

In some embodiments, binding of an ADC to the epitope of the cell-surface lineage-specific protein (e.g., CD33) induces internalization of the ADC, and the drug (or toxin) may be released intracellularly. In some embodiments, binding of the ADC to the epitope of a cell-surface lineage-specific protein induces internalization of the toxin or drug, which allows the toxin or drug to kill the cells expressing the lineage-specific protein. In some embodiments, binding of the ADC to the epitope of a cell-surface lineage-specific protein induces internalization of the toxin or drug, which may regulate the activity of the cell expressing the lineage-specific protein. The type of toxin or drug used in an ADC described herein is not limited to any specific type.

Toxins or drugs compatible for use in ADCs are known in the art and will be evident to one of ordinary skill in the art. See, e.g., Peters et al. Biosci. Rep. (2015) 35(4):e00225; Beck et al. Nat Rev Drug Disc (2017) 16:315-337; Marin-Acevedo et al. J. Hematol. Oncol. (2018) 11:8; Elgundi et al. Advanced Drug Delivery Reviews (2017) 122:2-19.

In some embodiments, an ADC may further comprise a linker (e.g., a peptide linker, such as a cleavable linker) attaching the antibody and drug molecule.

In some embodiments, a cytotoxic agent of the disclosure is gemtuzumab ozogamicin. Gemtuzumab ozogamicin is a recombinant, humanized anti-CD33 monoclonal antibody (IgG4 K antibody hP67.6) linked with (covalently attached to) the cytotoxic antitumor antibiotic calicheamicin (N-acetyl-y-calicheamicin) via a bifunctional linker (4-(4-acetylphenoxy) butanoic acid). Gemtuzumab ozogamicin is available commercially as Mylotarg® (Wyeth Pharmaceuticals, Philadelphia, Pa.). The antibody portion of gemtuzumab ozogamicin, referred to as hP67.6, binds specifically to the CD33 antigen.

Gemtuzumab ozogamicin contains amino acid sequences of which approximately 98.3% are of human origin. The constant region and framework regions contain human sequences while the complementarity-determining regions are derived from a murine antibody (P67.6) that binds CD33. This antibody is linked to N-acetyl-gamma calicheamicin via a bifunctional linker. Gemtuzumab ozogamicin has approximately 50% of the antibody loaded with 4-6 moles calicheamicin per mole of antibody. The remaining 50% of the antibody is not linked to the calicheamicin derivative. Gemtuzumab ozogamicin has a molecular weight of 151 to 153 kDa. Gemtuzumab ozogamicin and methods for making it are described in U.S. Pat. Nos. 5,733,001; 5,739,116; 5,767,285; 5,877,296; 5,606,040; and 5,714,586, which are incorporated by reference herein in their entirety.

Amino acid sequence of the heavy chain sequence
of gemtuzumab ozogamicin/Mylotarg ®
(SEQ ID NO: 26)
EVQLVQSGAEVKKPGSSVKVSCKASGYTITDSNIHWVRQAPGQSLEWIGY
IYPYNGGTDYNQKFKNRATLTVDNPTNTAYMELSSLRSEDTAFYYCVNGN
PWLAYWGQGTLVTVSSASTKGPSVFPLAPCSRSTSESTAALGCLVKDYFP
EPVTVSWNSGALTSGVHTFPAVLQSSGLYSLSSVVTVPSSSLGTKTYTCN
VDHKPSNTKVDKRVESKYGPPCPPCPAPEFLGGPSVFLFPPKPKDTLMIS
RTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK
Amino acid sequence of the light chain sequence
of gemtuzumab ozogamicin/Mylotarg ®
(SEQ ID NO: 27)
DIQLTQSPSTLSASVGDRVTITCRASESLDNYGIRFLTWFQQKPGKAPKL
LMYAASNQGSGVPSRFSGSGSGTEFTLTISSLQPDDFATYYCQQTKEVPW
SFGQGTKVEVKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSENRGEC

The anti-CD33 antibody binding domain for use in constructing the cytotoxic agent that targets CD33 as described herein may comprise the same heavy chain and/or light chain CDR regions as those in SEQ ID NO: 26 and SEQ ID NO: 27. Such antibodies may comprise amino acid residue variations in one or more of the framework regions. In some instances, the anti-CD33 antibody fragment may comprise a heavy chain variable region that shares at least 70% sequence identity (e.g., 75%, 80%, 85%, 90%, 95%, or higher) with the heavy chain variable region of gemtuzumab ozogamicin (provided in the heavy chain sequence of SEQ ID NO: 26) and/or may comprise a light chain variable region that shares at least 70% sequence identity (e.g., 75%, 80%, 85%, 90%, 95%, or higher) with the light chain variable region of gemtuzumab ozogamicin (provided in the light chain sequence of SEQ ID NO: 27).

CD33 is also a target of the anti-CD33 immunotoxin vadastuximab taurine (also referred to as SGN-CD33A, 33A) (Seattle Genetics). SGN-CD33A is an antibody-drug conjugate that may reduce multidrug resistance observed in response to treatment with gemtuzumab ozogamicin. In some embodiments, SGN-CD33A is used to treat the subject. In some embodiments, gemtuzumab ozogamicin and SGN-CD33A are used in combination, e.g., simultaneously or sequentially, to treat the subject. In some embodiments, the subject is also treated with chemotherapy.

Additional examples of ADCs include, without limitation, brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDS0780A, SGN-LIV1A, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Axl-ADC, pinatuzumab veodtin/RG7593/DCDT2980S, lifastuzumab vedotin/RG7599/DNIB0600A, indusatumab vedotin/MLN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BIIB015, MLN2704, AMG 172, AMG 595, LOP 628, vadastuximab talirine/SGN-CD33A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MEDI3726/ADC-401, IMGN779, IMGN632, gemtuzumab ozogamicin, inotuzumab ozogamicin/CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/BAY1129980, aprutumab ixadotin/BAY1187982, ARX788, AGS62P1, XMT-1522, AbGn-107, MEDI4276, and DSTA4637S/RG7861.

### Methods of Treatment and Administration

Aspects of the present disclosure provide methods involving administering to a subject an effective amount of a population of genetically engineered hematopoietic cells (also referred to herein as eHSCs or eHSPCs), as described herein, and an effective amount of a cytotoxic agent that comprises an anti-CD33 antigen binding domain. In some embodiments, the subject is diagnosed with a hematopoietic malignancy and is directed to undergo a combination treatment, involving administration of a population of genetically engineered hematopoietic cells, as described herein, and an effective amount of a cytotoxic agent that comprises an anti-CD33 antigen binding domain. As will be understood by one of ordinary skill in the art, a combination treatment involves more than one aspect of the treatment that may be performed together (e.g., administered at the same time or in a single composition) but also encompasses more than one treatment within a treatment regimen aimed to treat the malignancy, or any symptom or manifestation thereof. For example, some methods described herein involve the combination treatment to treat a hematopoietic malignancy (e.g., acute myeloid leukemia) involving administering an effective amount of a population of genetically engineered hematopoietic cells, as described herein, and an effective amount of a cytotoxic agent that comprises an anti-CD33 antigen binding domain. In some embodiments, the methods described herein involve the combination treatment to treat a premalignant stage of a hematopoietic malignancy (e.g., myelodysplastic syndrome (MDS)) involving administering an effective amount of a population of genetically engineered hematopoietic cells, as described herein, and an effective amount of a cytotoxic agent that comprises an anti-CD33 antigen binding domain. Some combination treatment methods provided herein comprise sequential administration of a population of genetically engineered hematopoietic cells (e.g., CD33KO eHSPCs) and the cytotoxic agent (e.g., Mylotarg®), including, for example, administration of the cytotoxic agent (e.g., Mylotarg®) after administration of the genetically engineered hematopoietic cells. The therapeutic modalities of the combination treatments provided herein, e.g., the population of genetically engineered hematopoietic cells and the cytotoxic agent, may be administered according to the same or according to different dosing regimens (including dosing frequencies, amounts, administration routes) as part of the combination treatment, and such dosing regimens may overlap in time or be sequential.

In some embodiments, an effective number of genetically engineered hematopoietic stem cells, e.g., CD33-modified hematopoietic stem cells described herein, is administered in combination with a cytotoxic agent comprising an anti-CD33 antigen-binding domain (e.g., an anti-CD33 cancer therapy, such as gemtuzumab ozogamicin/Mylotarg®). In some embodiments, an effective number of cells comprising a modified CD33 and a modified second lineage-specific cell surface antigen are administered in combination with a cytotoxic agent. In some embodiments, the cytotoxic agent comprises an antibody, an ADC, or an immune cell expressing a chimeric antigen receptor (CAR). In some embodiments, the cytotoxic agent comprises gemtuzumab ozogamicin.

In some embodiments, an effective amount of a population of genetically engineered hematopoietic stem cells, e.g., CD33-modified hematopoietic stem cells described herein, comprises about 10² cells/kilogram to about 10¹⁰ cells/kilogram body weight of a subject. In some embodiments, an effective amount of a population of genetically engineered hematopoietic stem cells, e.g., CD33-modified hematopoietic stem cells described herein, comprises about 10⁴ cells/kilogram to about 10⁸ cells/kilogram body weight of a subject. In some embodiments, an effective amount of a population of genetically engineered hematopoietic stem cells, e.g., CD33-modified hematopoietic stem cells described herein, comprises about 10⁶ cells/kilogram to about 10⁸ cells/kilogram body weight of a subject. In some embodiments, an effective amount of a population of genetically engineered hematopoietic stem cells, e.g., CD33-modified hematopoietic stem cells described herein, comprises about 10⁵ cells/kilogram to about 10⁷ cells/kilogram body weight of a subject. In some embodiments, an effective amount of a population of genetically engineered hematopoietic stem cells, e.g., CD33-modified hematopoietic stem cells described herein, comprises about 10⁶ cells/kilogram to about 10⁷ cells/kilogram body weight of a subject. In some embodiments, an effective amount of a population of genetically engineered hematopoietic stem cells, e.g., CD33-modified hematopoietic stem cells described herein, comprises about 10⁵ cells/kilogram, about 10⁶ cells/kilogram, about 10⁷ cells/kilogram, or about 10⁸ cells/kilogram body weight of a subject. In some embodiments, an effective amount a population of genetically engineered hematopoietic stem cells, e.g., CD33-modified hematopoietic stem cells described herein, comprises at least 10² cells, at least 10³ cells, at least 10⁴ cells, at least 10⁵ cells, at least 5×10⁵ cells, at least 10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, at least 1×10⁷ cells, or multiples thereof.

In some embodiments, an effective amount of a population of genetically engineered hematopoietic stem cells, e.g., CD33-modified hematopoietic stem cells described herein, comprises about 1.0×10⁵, about 2.0×10⁵, about 3.0×10⁵, about 4.0×10⁵, about 5.0×10⁵, about 6.0×10⁵, about 7.0×10⁵, about 8.0×10⁵, about 9.0×10⁵, about 1.0×10⁶, about 2.0×10⁶, about 3.0×10⁶, about 4.0×10⁶, about 5.0×10⁶, about 6.0×10⁶, about 7.0×10⁶, about 8.0×10⁶, about 9.0×10⁶, about 1.0×10⁷, about 2.0×10⁷, about 3.0×10⁷, about 4.0×10⁷, 5.0×10⁷, about 6.0×10⁷, about 7.0×10⁷, about 8.0×10⁷, about 9.0×10⁷, or about 1.0×10⁸ cells/kilogram body weight of a subject. In some embodiments, an effective amount of a population of genetically engineered hematopoietic stem cells, e.g., CD33-modified hematopoietic stem cells described herein, comprises about 3.0×10⁶ cells/kilogram body weight of a subject.

Hematopoietic stem cells, e.g., CD34+ hematopoietic stem cells, that can, at least in some embodiments, serve as the starting material for generating the genetically engineered hematopoietic stem cells, e.g., CD33-modified hematopoietic stem cells described herein, can be derived from one or more donors or can be obtained from an autologous source. In some embodiments, the genetically engineered hematopoietic stem cells, e.g., CD33-modified hematopoietic stem cells described herein, are expanded in culture prior to administration to a subject in need thereof.

A typical number of cells, e.g., immune cells or hematopoietic cells, administered to a mammal (e.g., a human) can be, for example, in the range of one million to 100 billion cells; however, amounts below or above this exemplary range are also within the scope of the present disclosure.

In some embodiments, a cytotoxic agent, e.g., gemtuzumab ozogamicin, is used in a therapeutically effective amount, e.g., in combination with the population of genetically engineered hematopoietic stem cells, e.g., CD33-modified hematopoietic stem cells described herein. In some embodiments, an effective amount of a cytotoxic agent (e.g., gemtuzumab ozogamicin) is about 0.01 mg/m² to about 3.0 mg/m² body surface area of a subject. In some embodiments, an effective amount of a cytotoxic agent is about 0.05 mg/m² to about 2.5 mg/m², about 0.1 mg/m² to about 2.0 mg/m², about 0.1 mg/m² to about 1.0 mg/m², about 1.0 mg/m² to about 2.0 mg/m², or about 1.5 mg/m² to about 2.5 mg/m² body surface area of a subject. In some embodiments, an effective amount of a cytotoxic agent is about 0.05 mg/m², about 0.1 mg/m², about 0.25 mg/m², about 0.5 mg/m², about 1.0 mg/m², about 1.5 mg/m², about 2.0 mg/m², or about 2.5 mg/m² body surface area of a subject. In some embodiments, an effective amount of a cytotoxic agent is about 2.0 mg/m² body surface area of a subject.

In some embodiments, gemtuzumab ozogamicin is used in a therapeutically effective amount, e.g., in combination with a population of genetically engineered hematopoietic stem cells, e.g., CD33-modified hematopoietic stem cells described herein. In some embodiments, an effective amount of gemtuzumab ozogamicin is about 0.01 mg/m² to about 3.0 mg/m² body surface area of a subject. In some embodiments, an effective amount of gemtuzumab ozogamicin is about 0.05 mg/m² to about 2.5 mg/m², about 0.1 mg/m² to about 2.0 mg/m², about 0.1 mg/m² to about 1.0 mg/m², about 1.0 mg/m² to about 2.0 mg/m², or about 1.5 mg/m² to about 2.5 mg/m² body surface area of a subject. In some embodiments, an effective amount of gemtuzumab ozogamicin is about 0.05 mg/m², about 0.1 mg/m², about 0.25 mg/m², about 0.5 mg/m², about 1.0 mg/m², about 1.5 mg/m², about 2.0 mg/m², or about 2.5 mg/m² body surface area of a subject. In some embodiments, an effective amount of gemtuzumab ozogamicin is about 2.0 mg/m² body surface area of a subject.

In some embodiments, an effective amount of a population of genetically engineered hematopoietic cells, e.g. CD33-modified hematopoietic stem cells described herein, is about 4 cells/kilogram to about 10⁸ cells/kilogram body weight of a subject, and an effective amount of a cytotoxic agent (e.g., gemtuzumab ozogamicin) is about 0.01 mg/m² to about 3.0 mg/m² body surface area of the subject. In some embodiments, an effective amount of a population of genetically engineered hematopoietic cells, e.g. CD33-modified hematopoietic stem cells described herein, is about 10⁶ cells/kilogram to about 10⁷ cells/kilogram body weight of a subject, and an effective amount of the cytotoxic agent is about 0.1 mg/m² to about 2.0 mg/m² body surface area of the subject. In some embodiments, an effective amount of a population of genetically engineered hematopoietic cells, e.g. CD33-modified hematopoietic stem cells described herein, is about 3.0×10⁶ cells/kilogram body weight of a subject, and an effective amount of the cytotoxic agent is about 0.1 mg/m², about 0.25 mg/m², about 0.5 mg/m², about 1.0 mg/m², or about 2.0 mg/m² body surface area of the subject. In some embodiments, an effective amount of a population of genetically engineered hematopoietic cells, e.g. CD33-modified hematopoietic stem cells described herein, is about 3.0×10⁶ cells/kilogram body weight of a subject, and an effective amount of the cytotoxic agent is about 2.0 mg/m² body surface area of the subject.

In some embodiments, an effective amount of a population of genetically engineered hematopoietic cells, e.g. CD33-modified hematopoietic stem cells described herein, is about 4 cells/kilogram to about 10⁸ cells/kilogram body weight of a subject, and an effective amount of gemtuzumab ozogamicin is about 0.01 mg/m² to about 3.0 mg/m² body surface area of the subject. In some embodiments, an effective amount of a population of genetically engineered hematopoietic cells, e.g. CD33-modified hematopoietic stem cells described herein, is about 10⁶ cells/kilogram to about 10⁷ cells/kilogram body weight of a subject, and an effective amount of gemtuzumab ozogamicin is about 0.1 mg/m² to about 2.0 mg/m² body surface area of the subject. In some embodiments, an effective amount of a population of genetically engineered hematopoietic cells, e.g. CD33-modified hematopoietic stem cells described herein, is about 3.0×10⁶ cells/kilogram body weight of a subject, and an effective amount of gemtuzumab ozogamicin is about 0.1 mg/m², about 0.25 mg/m², about 0.5 mg/m², about 1.0 mg/m², or about 2.0 mg/m² body surface area of the subject. In some embodiments, an effective amount of a population of genetically engineered hematopoietic cells, e.g. CD33-modified hematopoietic stem cells described herein, is about 3.0×10⁶ cells/kilogram body weight of a subject, and an effective amount of gemtuzumab ozogamicin is about 2.0 mg/m² body surface area of the subject.

In some embodiments, a population of genetically engineered hematopoietic cells and a cytotoxic agent (e.g., gemtuzumab ozogamicin) are administered at the same time or different times in temporal proximity. As used herein, in some embodiments, temporal proximity refers to the timing of the administration of the population of genetically engineered hematopoietic cells relative to the administration of a cytotoxic agent. It should be appreciated that no particular ordering is implied in the use of this terminology unless an ordering is expressly stated. For example, administration of the population of genetically engineered hematopoietic cells and a cytotoxic agent in temporal proximity can include administration of the hematopoietic cells prior to, following, or at approximately the same time as the administration of the cytotoxic agent. Furthermore, the treatments may be admixed or in separate volumes. For example, in some embodiments, administration in combination includes administration in the same course of treatment, e.g., in the course of treating a cancer with an anti-CD33 therapy, the subject may be administered an effective number of CD33-modified cells concurrently or sequentially, e.g., before, during, or after the treatment, with the cytotoxic agent.

In some embodiments, administering in temporal proximity comprises administering a population of genetically engineered hematopoietic stem cells and a cytotoxic agent within a single treatment regimen. In some embodiments, administering in temporal proximity comprises administering a population of genetically engineered hematopoietic stem cells and a cytotoxic agent simultaneously or concurrently. In some embodiments, administering in temporal proximity comprises administering a population of genetically engineered hematopoietic stem cells and a cytotoxic agent sequentially (e.g., administering either treatment before the other treatment). In some embodiments, a population of genetically engineered hematopoietic stem cells is administered prior to a cytotoxic agent. In some embodiments, administering in temporal proximity comprises administering a population of genetically engineered hematopoietic stem cells within 120 days (e.g., within 90 days, within 60 days, within 30 days, within 20 days, within 10 days, within 7 days, or within 1 day) of administering a cytotoxic agent. In some embodiments, administering in temporal proximity comprises administering a population of genetically engineered hematopoietic stem cells to a subject within 120 days (e.g., within 90 days, within 60 days, within 30 days, within 20 days, within 10 days, within 7 days, or within 1 day) prior to administering a cytotoxic agent to the subject. In some embodiments, administering in temporal proximity comprises administering a population of genetically engineered hematopoietic stem cells to a subject within 120 days (e.g., within 90 days, within 60 days, within 30 days, within 20 days, within 10 days, within 7 days, or within 1 day) after administering a cytotoxic agent to the subject.

In some embodiments, the subject is evaluated based on one or more parameters, such as level of engraftment, following administration of the population of genetically engineered hematopoietic cells, and described herein, prior to administration of the cytotoxic agent. In some embodiments, the subject has a CD33-negative absolute neutrophil count (ANC) above a threshold value (e.g. at least 1000/dL) prior to administration of the cytotoxic agent.

In some embodiments, a cytotoxic agent is administered in multiple doses, for example at a regular interval (e.g., every week, every two weeks, every three weeks, every four weeks, every month, every two months, every three months, every four months, every five months, or every six months). In some embodiments, a cytotoxic agent is administered in multiple doses of the effective amount every four weeks. For example, in some embodiments, an effective amount of the cytotoxic agent is administered in a first dose, which may be followed by one or more subsequent doses of the effective amount, where each dose is separated by approximately four weeks (e.g., 28 days). In some embodiments, each dose is separated by about two weeks to about six weeks (e.g., about two weeks, about three weeks, about four weeks, about five weeks, about six weeks, about three weeks to about five weeks, or about four weeks to about six weeks). In some embodiments, an effective amount of the cytotoxic agent is administered to a subject in at least one dose, at least two doses, at least three doses, between one and six doses, between one and four doses, between one and three doses, or four doses. In some embodiments, each dose of the cytotoxic agent is about 2.0 mg/m². In some embodiments, the cytotoxic agent is administered in multiple doses of about 2.0 mg/m² every four weeks.

In some embodiments, a subject in need of treatment in accordance with the present disclosure has been identified as having newly-diagnosed, de novo CD33-positive AML. When given as part of a combination regimen for the treatment of newly-diagnosed, de novo CD33-positive AML, the recommended treatment course including gemtuzumab ozogamicin consists of 1 induction cycle and 2 consolidation cycles. For the induction cycle, the recommended dose of gemtuzumab ozogamicin is 3 mg/m² (up to one 4.5 mg vial) on days 1, 4, and 7 in combination with daunorubicin and cytarabine. For subjects requiring a second induction cycle, gemtuzumab ozogamicin is not administered during the second induction cycle. For the consolidation cycles, the recommended dose of gemtuzumab ozogamicin is 3 mg/m² on day 1 (up to one 4.5 mg vial) in combination with daunorubicin and cytarabine.

In some embodiments, a subject in need of treatment in accordance with the present disclosure has been identified as having newly-diagnosed CD33-positive AML. When given as a single agent therapy for the treatment of newly-diagnosed CD33-positive AML, the recommended treatment course of gemtuzumab ozogamicin consists of 1 cycle of induction and up to 8 cycles of continuation therapy. For the induction cycle, the recommended dose of gemtuzumab ozogamicin is 6 mg/m² as a single agent on day 1 and 3 mg/m² on day 8. For continuation, the recommended dose of gemtuzumab ozogamicin is 2 mg/m² as a single agent on day 1 every 4 weeks.

In some embodiments, a subject in need of treatment in accordance with the present disclosure has been diagnosed as having, or is suspected of having, relapsed or refractory CD33-positive AML. When given as a single agent therapy for the treatment of relapsed or refractory CD33-positive AML, the recommended dose of gemtuzumab ozogamicin is 3 mg/m² (up to one 4.5 mg vial) on days 1, 4, and 7.

In some embodiments, a subject is pretreated with one or more of a corticosteroid, antihistamine, and acetaminophen prior to administration of gemtuzumab ozogamicin. In some embodiments, the subject is pretreated approximately 1 hour (e.g., about 30 minutes to 1.5 hours, about 45 minutes to 1.5 hours, about 1 to 2 hours, or about 45 minutes to 1 hour) prior to administration of gemtuzumab ozogamicin. In some embodiments, a subject is pretreated with approximately 650 mg acetaminophen (e.g., orally) and approximately 50 mg diphenhydramine (e.g., orally or intravenously) 1 hour prior to administration of gemtuzumab ozogamicin. In some embodiments, a subject is pretreated with approximately 1 mg/kg methylprednisolone or an equivalent dose of an alternative corticosteroid within 30 minutes prior to administration of gemtuzumab ozogamicin. Pediatric subjects may be pretreated with acetaminophen 15 mg/kg (maximum of 650 mg), diphenhydramine 1 mg/kg (maximum of 50 mg), and 1 mg/kg methylprednisolone orally or intravenously; additional doses of acetaminophen and diphenhydramine may be administered every 4 hours after the initial pretreatment dose. Pretreatment may be repeated with the same dose of methylprednisolone or an equivalent corticosteroid for any sign of an infusion reaction, such as fever, chills, hypotension, or dyspnea during the infusion or within 4 hours afterwards.

In some embodiments, the subject does not have a homozygous dominant genotype for CD33 single nucleotide polymorphism (SNP) rs12459419. In some embodiments, the subject does not have acute promyelocytic leukemia or chronic myeloid leukemia. In some embodiments, the subject does not have a genetic translocation associated with acute promyelocytic leukemia or chronic myeloid leukemia, optionally wherein the genetic translocation is t(15; 17)(q22; q21) or t(9; 22)(q34; q11). In some embodiments, the subject has not previously received autologous or allogeneic stem cell transplantation. In some embodiments, the subject has not previously received the cytotoxic agent.

In some embodiments, the method further comprises determining a percent donor chimerism and/or a level of CD33-negative myeloid hematopoiesis in a peripheral blood sample from the subject.

In some embodiments, gemtuzumab ozogamicin is reconstituted from a lyophilized form prior to administration. In some embodiments, the lyophilized form comprises approximately 4.5 mg of a lyophilized cake or powder. In some embodiments, the lyophilized form comprises a lyophilized cake or powder in a single-dose vial for reconstitution and/or dilution.

In some embodiments, one or more other antibodies that selectively bind CD33, or antigen binding fragments thereof, may be used to treat the subject. In some embodiments, an antibody or an antigen binding fragment thereof that selectively binds to CD33 is linked to a toxin to target CD33 expressing cancer cells in a subject. Any antibody that selectively binds CD33 may be used.

In some embodiments, a subject has been preconditioned prior to administration of a cytotoxic agent and/or a population of genetically engineered hematopoietic stem cells. In some embodiments, preconditioning of a subject comprises administering one or more chemotherapeutic agents to the subject. Examples of chemotherapeutic agents include, without limitation, busulfan, melphalan, fludarabine, cyclophosphamide, and thiotepa. In some embodiments, preconditioning comprises total body irradiation of a subject. In some embodiments, preconditioning comprises administering antibodies that bind human T cells (e.g., rabbit anti-thymocyte globulins (rATG)). In some embodiments, preconditioning occurs within two weeks (e.g., within 14 days, within 12 days, within 10 days, within 9 days, within 7 days) prior to administration of a cytotoxic agent and/or hematopoietic cells. In some embodiments, preconditioning occurs over a period of about one day to about ten days. In some embodiments, preconditioning occurs over a period of about nine days.

In some embodiments, a composition of the disclosure (e.g., a population of hematopoietic cells, a cytotoxic agent) may be administered via a route such as, but not limited to, enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intraarticular, intrabiliary, intrabronchial, intrabursal, intracartilaginous (within a cartilage), intracaudal (within the cauda equine), intraci sternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis and spinal. As will be appreciated by one of ordinary skill in the art, administration of the population of genetically engineered hematopoietic cells and the cytotoxic agent may be performed by the same administration route (e.g., intravenous infusion) or by different administration routes.

Modes of administration include injection, infusion, instillation, and/or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some examples, the route is intravenous. For the delivery of cells, administration by injection or infusion can be made. In some embodiments, a population of genetically engineered hematopoietic stem cells can be administered systemically. The phrases “systemic administration,” “administered systemically”, “peripheral administration” and “administered peripherally” refer to the administration of a population of progenitor cells other than directly into a target site, tissue, or organ, such that it enters, instead, the subject's circulatory system and, thus, is subject to metabolism and other like processes.

The efficacy of a treatment having a composition for the treatment of a hematopoietic malignancy (e.g., AML) can be determined by the skilled clinician. However, a treatment is considered “effective treatment,” if any one or all of the signs or symptoms of the hematopoietic malignancy are altered in a beneficial manner, or other clinically accepted symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

Also provided herein are clinical preparations comprising populations of cells comprising any of the genetically modified hematopoietic cells, or descendants thereof, described herein. In some embodiments, the composition comprises a population of at least 1×10⁶ cells per milliliter (mL) in a medium, wherein the population of cells comprise genetically modified hematopoietic cells, or descendants thereof, comprising a modified gene encoding CD33 that is engineered to have reduced or eliminated expression of a CD33 antigen. In some embodiments, the population comprises at least 2×10⁶ cells per mL, at least 3×10⁶ cells per mL, at least 4×10⁶ cells per mL, at least 5×10⁶ cells per mL, at least 6×10⁶ cells per mL, at least at least 7×10⁶ cells per mL, at least 8×10⁶ cells per mL, or at least 9×10⁶ cells per mL.

In some embodiments, the medium has a volume between about 5-150 mL. In some embodiments, the medium has a volume between about 10-100 mL. In some embodiments, the medium has a volume between about 25-75 mL. In some embodiments, the medium has a volume between about 30-70 mL. In some embodiments, the medium has a volume between about 40-60 mL. In some embodiments, the medium has a volume of about 45 mL. In some embodiments, the medium has a volume of about 30 mL. In some embodiments, the medium has a volume of about 35 mL. In some embodiments, the medium has a volume of about 40 mL. In some embodiments, the medium has a volume of about 50 mL. In some embodiments, the medium has a volume of about 55 mL. In some embodiments, the medium has a volume of about 60 mL. In some embodiments, the medium has a volume of about 70 mL. In some embodiments, the medium has a volume between about 40-50 mL. In some embodiments, the medium has a volume of about 40 mL, 41 mL, 42 mL, 43 mL, 44 mL, 45 mL, 46 mL, 47 mL, 48 mL, 49 mL, or about 50 mL. In some embodiments, the medium has a volume of about 45 mL.

In some embodiments, the composition comprises a population of between about 1×6-1×10⁸ cells total in the medium. In some embodiments, the composition comprises a population of about 1×10⁷, 2×10⁷, 3×10⁷, 4×10⁷, 5×10⁷, 6×10⁷, 7×10⁷, 8×10⁷, 9×10⁷, or 1×10⁸ cells total in the medium. In some embodiments, the population comprises at least 0.5×10⁶ cells per mL, at least 1×10⁶ cells per mL, at least 2×10⁶ cells per mL, at least 3×10⁶ cells per mL, at least 4×10⁶ cells per mL, at least 5×10⁶ cells per mL, at least 6×6 cells per mL, at least at least 7×10⁶ cells per mL, at least 8×10⁶ cells per mL, or at least 9×10⁶ cells per mL. In some embodiments, the population comprises at least 0.5×10⁶ cells per mL, at least 1×10⁶ cells per mL, at least 2×10⁶ cells per mL, at least 3×10⁶ cells per mL, at least 4×10⁶ cells per mL, at least 5×10⁶ cells per mL, at least 6×10⁶ cells per mL, at least at least 7×10⁶ cells per mL, at least 8×10⁶ cells per mL, or at least 9×10⁶ cells per mL. In some embodiments, the cell population comprises at least 1×10⁹ viable cells, at least 2×10⁹ viable cells, at least 3×10⁹ viable cells, at least 4×10⁹ viable cells, at least 5×10⁹ viable cells, at least 6×10⁹ viable cells, at least 7×10⁹ viable cells, at least 8×10⁹ viable cells, at least 9×10⁹ viable cells, at least 1×10¹⁰ viable cells, at least 2×10¹⁰ viable cells, at least 3×10¹⁰ viable cells, at least 4×10¹⁰ viable cells, at least 5×10¹⁰ viable cells, at least 6×10¹⁰ viable cells, at least 7×10¹⁰ viable cells, at least 8×10¹⁰ viable cells, at least 9×10¹⁰ viable cells, or at least 1×10¹¹ viable cells, wherein, in some embodiments, at least 50%, at least 60%, at least 70%, at least 80%, at least 90%, at least 95%, at least 97%, at least 98%, or at least 99% of the cells of the population are genetically modified hematopoietic cells, or descendants thereof, having reduced or eliminated expression of a CD33 antigen.

In some embodiments, at least 50%, at least 55%, at least 60%, at least 65%, at least 70%, at least 75%, at least 80%, at least 85%, at least 90%, at least 95%, or at least 99% of the cells of population are genetically modified hematopoietic cells, or descendants thereof, having reduced or eliminated expression of a CD33 antigen.

In some embodiments, the medium is a cryopreservation medium comprising a cryoprotectant. Non-limiting examples of cryoprotects include cetamide, agarose, alginate, 1-analine, albumin, ammonium acetate, butanediol, chondroitin sulfate, chloroform, choline, diethylene glycol, dimethyl acetamide, dimethyl formamide, dimethylsulfoxide (DMSO), erythritol, ethanol, ethylene glycol, formamide, glucose, glycerol, a-glycerol phosphate, glycerol monoacetate, glycine, hydroxyethyl starch, inositol, lactose, magnesium chloride, magnesium sulfate, maltose, mannitol, Mannose, methanol, methyl acetamide, methylformamide, methyl urea, phenol, pluronic polyol, polyethylene glycol, polyvinylpyrrolidone, proline, propylene glycol, pyridine N-oxide, ribose, serine, sodium bromide, chloride Sodium, sodium iodide, sodium nitrate, sodium sulfate, sorbitol, sucrose, trehalose, triethylene glycol, trimethylamine acetate, urea, valine, and xylose In some embodiments, the cryoprotectant comprises dimethylsulfoxide (DMSO). In some embodiments, the cryoprotectant comprises DMSO in an amount of about 10%. (v/v).

In some embodiments, the composition is in a frozen state. In some embodiments, the composition has been subjected to a cryopreservation process. As will be evident one of ordinary skill in the art, cryopreservation processes are methods aimed, for example, to preserve (viable) cells by cooling and storing a sample comprising the cells at a low temperature (e.g., at or below −80 C. In some embodiments, the cryopreservation process is controlled-rate freezing.

### Genomic Editing

Aspects of the present disclosure relate to populations of genetically engineered hematopoietic cells, or descendants thereof, comprising a modified gene encoding CD33 that is engineered to have reduced or eliminated expression of a CD33 antigen. The gene encoding CD33 may be engineered by any means known in the art such that the cell has reduced or eliminated expression of a CD33 antigen.

The term “binds”, as used herein with reference to a gRNA interaction with a target domain, refers to the gRNA molecule and the target domain forming a complex. The complex may comprise two strands forming a duplex structure, or three or more strands forming a multi-stranded complex. The binding may constitute a step in a more extensive process, such as the cleavage of the target domain by a Cas endonuclease. In some embodiments, the gRNA binds to the target domain with perfect complementarity, and in other embodiments, the gRNA binds to the target domain with partial complementarity, e.g., with one or more mismatches. In some embodiments, when a gRNA binds to a target domain, the full targeting domain of the gRNA base pairs with the targeting domain. In other embodiments, only a portion of the target domain and/or only a portion of the targeting domain base pairs with the other. In an embodiment, the interaction is sufficient to mediate a target domain-mediated cleavage event.

A “Cas9 molecule” as that term is used herein, refers to a molecule or polypeptide that can interact with a gRNA and, in concert with the gRNA, home or localize to a site which comprises a target domain. Cas9 molecules include naturally occurring Cas9 molecules and engineered, altered, or modified Cas9 molecules that differ, e.g., by at least one amino acid residue, from a naturally occurring Cas9 molecule.

The terms “gRNA” and “guide RNA” are used interchangeably throughout and refer to a nucleic acid that promotes the specific targeting or homing of a gRNA/Cas9 molecule complex to a target nucleic acid. A gRNA can be unimolecular (having a single RNA molecule), sometimes referred to herein as sgRNAs, or modular (comprising more than one, and typically two, separate RNA molecules). A gRNA may bind to a target domain in the genome of a host cell. The gRNA (e.g., the targeting domain thereof) may be partially or completely complementary to the target domain. The gRNA may also comprise a “scaffold sequence,” (e.g., a tracrRNA sequence), that recruits a Cas9 molecule to a target domain bound to a gRNA sequence (e.g., by the targeting domain of the gRNA sequence). The scaffold sequence may comprise at least one stem loop structure and recruits an endonuclease. Exemplary scaffold sequences can be found, for example, in Jinek, et al. Science (2012) 337(6096):816-821, Ran, et al. Nature Protocols (2013) 8:2281-2308, PCT Publication No. WO2014/093694, and PCT Publication No. WO2013/176772.

The term “mutation” is used herein to refer to a genetic change (e.g., insertion, deletion, or substitution) in a nucleic acid compared to a reference sequence, e.g., the corresponding wild-type nucleic acid. In some embodiments, a mutation to a gene detargetizes the protein produced by the gene. In some embodiments, a detargetized CD33 protein is not bound by, or is bound at a lower level by, an agent that targets CD33. The “targeting domain” of the gRNA is complementary to the “target domain” on the target nucleic acid. The strand of the target nucleic acid comprising the nucleotide sequence complementary to the core domain of the gRNA is referred to herein as the “complementary strand” of the target nucleic acid. Guidance on the selection of targeting domains can be found, e.g., in Fu Y et al, Nat Biotechnol (2014) 32: 279-284 (doi: 10.1038/nbt.2808) and Sternberg S H et al., Nature (2014) 507(7490): 62-7 (doi: 10.1038/nature13011).

### Nucleases

In some embodiments, a cell (e.g., HSC or HPC) described herein is made using a nuclease described herein. Exemplary nucleases include Cas molecules (e.g., Cas9 or Cas12a), TALENs, ZFNs, and meganucleases. In some embodiments, a nuclease is used in combination with a CD33 gRNA described herein (e.g., according to Table 3).

### Cas9 Molecules

In some embodiments, a CD33 gRNA described herein is complexed with a Cas9 molecule. Various Cas9 molecules can be used. In some embodiments, a Cas9 molecule is selected that has the desired PAM specificity to target the gRNA/Cas9 molecule complex to the target domain in CD33. In some embodiments, genetically engineering a cell also comprises introducing one or more (e.g., 1, 2, 3 or more) Cas9 molecules into the cell.

Cas9 molecules of a variety of species can be used in the methods and compositions described herein. In embodiments, the Cas9 molecule is of, or derived from, S. pyogenes (SpCas9), S. aureus (SaCas9) or S. thermophilus. Additional suitable Cas9 molecules include those of, or derived from, Staphylococcus aureus, Neisseria meningitidis (NmCas9), Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni (CjCas9), Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae.

In some embodiments, the Cas9 molecule is a naturally occurring Cas9 molecule. In some embodiments, the Cas9 molecule is an engineered, altered, or modified Cas9 molecule that differs, e.g., by at least one amino acid residue, from a reference sequence, e.g., the most similar naturally occurring Cas9 molecule or a sequence of Table 50 of PCT Publication No. WO2015/157070, which is herein incorporated by reference in its entirety.

A naturally occurring Cas9 molecule typically comprises two lobes: a recognition (REC) lobe and a nuclease (NUC) lobe; each of which further comprises domains described, e.g., in PCT Publication No. WO 2015/157070, e.g., in FIGS. 9A-9B therein, which is incorporated herein by reference in its entirety.

The REC lobe comprises the arginine-rich bridge helix (BH), the REC1 domain, and the REC2 domain. The REC lobe appears to be a Cas9-specific functional domain. The BH domain is a long alpha helix and arginine rich region and comprises amino acids 60-93 of the sequence of S. pyogenes Cas9. The REC1 domain is involved in recognition of the repeat:anti-repeat duplex, e.g., of a gRNA or a tracrRNA. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat: anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule and comprises amino acids 1099-1368 of the sequence of S. pyogenes Cas9.

Crystal structures have been determined for naturally occurring bacterial Cas9 molecules (Jinek et al., Science (2014) 343(6176): 1247997) and for S. pyogenes Cas9 with a guide RNA (e.g., a synthetic fusion of crRNA and tracrRNA) (Nishimasu et al., Cell (2014) 156:935-949; and Anders et al., Nature (2014) doi: 10.1038/nature13579).

In some embodiments, a Cas9 molecule described herein has nuclease activity, e.g., double strand break activity. In some embodiments, the Cas9 molecule has been modified to inactivate one of the catalytic residues of the endonuclease. In some embodiments, the Cas9 molecule is a nickase and produces a single stranded break. See, e.g., Dabrowska et al. Frontiers in Neuroscience (2018) 12(75). It has been shown that one or more mutations in the RuvC and HNH catalytic domains of the enzyme may improve Cas9 efficiency. See, e.g., Sarai et al. Currently Pharma. Biotechnol. (2017) 18(13). In some embodiments, the Cas9 molecule is fused to a second domain, e.g., a domain that modifies DNA or chromatin, e.g., a deaminase or demethylase domain. In some such embodiments, the Cas9 molecule is modified to eliminate its endonuclease activity.

In some embodiments, a Cas9 molecule described herein is administered together with a template for homology directed repair (HDR). In some embodiments, a Cas9 molecule described herein is administered without an HDR template.

In some embodiments, the Cas9 molecule is modified to enhance specificity of the enzyme (e.g., reduce off-target effects, maintain robust on-target cleavage). In some embodiments, the Cas9 molecule is an enhanced specificity Cas9 variant (e.g., eSPCas9). See, e.g., Slaymaker et al. Science (2016) 351 (6268): 84-88. In some embodiments, the Cas9 molecule is a high fidelity Cas9 variant (e.g., SpCas9-HF1). See, e.g., Kleinstiver et al. Nature (2016) 529: 490-495.

Various Cas9 molecules are known in the art and may be obtained from various sources and/or engineered/modified to modulate one or more activities or specificities of the enzymes. In some embodiments, the Cas9 molecule has been engineered/modified to recognize one or more PAM sequence. In some embodiments, the Cas9 molecule has been engineered/modified to recognize one or more PAM sequence that is different than the PAM sequence the Cas9 molecule recognizes without engineering/modification. In some embodiments, the Cas9 molecule has been engineered/modified to reduce off-target activity of the enzyme.

In some embodiments, the nucleotide sequence encoding the Cas9 molecule is modified further to alter the specificity of the endonuclease activity (e.g., reduce off-target cleavage, decrease the endonuclease activity or lifetime in cells, increase homology-directed recombination and reduce non-homologous end joining). See, e.g., Komor et al. Cell (2017) 168: 20-36. In some embodiments, the nucleotide sequence encoding the Cas9 molecule is modified to alter the PAM recognition of the endonuclease. For example, the Cas9 molecule SpCas9 recognizes PAM sequence NGG, whereas relaxed variants of the SpCas9 comprising one or more modifications of the endonuclease (e.g., VQR SpCas9, EQR SpCas9, VRER SpCas9) may recognize the PAM sequences NGA, NGAG, NGCG. PAM recognition of a modified Cas9 molecule is considered “relaxed” if the Cas9 molecule recognizes more potential PAM sequences as compared to the Cas9 molecule that has not been modified. For example, the Cas9 molecule SaCas9 recognizes PAM sequence NNGRRT, whereas a relaxed variant of the SaCas9 comprising one or more modifications (e.g., KKH SaCas9) may recognize the PAM sequence NNNRRT. In one example, the Cas9 molecule FnCas9 recognizes PAM sequence NNG, whereas a relaxed variant of the FnCas9 comprising one or more modifications of the endonuclease (e.g., RHA FnCas9) may recognize the PAM sequence YG. In one example, the Cas9 molecule is a Cpf1 endonuclease comprising substitution mutations S542R and K607R and recognize the PAM sequence TYCV. In one example, the Cas9 molecule is a Cpf1 endonuclease comprising substitution mutations S542R, K607R, and N552R and recognize the PAM sequence TATV. See, e.g., Gao et al. Nat. Biotechnol. (2017) 35(8): 789-792.

In some embodiments, more than one (e.g., 2, 3, or more) Cas molecules, e.g., Cas9 molecules, are used. In some embodiments, at least one of the Cas9 molecule is a Cas9 enzyme. In some embodiments, at least one of the Cas molecules is a Cpf1 enzyme. In some embodiments, at least one of the Cas9 molecules is derived from Streptococcus pyogenes. In some embodiments, at least one of the Cas9 molecules is derived from Streptococcus pyogenes and at least one Cas9 molecules is derived from an organism that is not Streptococcus pyogenes.

In some embodiments, the Cas9 molecule is a base editor. Base editor endonuclease generally comprises a catalytically inactive Cas9 molecule fused to a function domain. See, e.g., Eid et al. Biochem. J. (2018) 475(11): 1955-1964; Rees et al. Nature Reviews Genetics (2018) 19:770-788. In some embodiments, the catalytically inactive Cas9 molecule is dCas9. In some embodiments, the catalytically inactive Cas9 molecule (dCas9) is fused to one or more uracil glycosylase inhibitor (UGI) domains. In some embodiments, the endonuclease comprises a dCas9 fused to an adenine base editor (ABE), for example an ABE evolved from the RNA adenine deaminase TadA. In some embodiments, the endonuclease comprises a dCas9 fused to cytidine deaminase enzyme (e.g., APOBEC deaminase, pmCDA1, activation-induced cytidine deaminase (AID)). In some embodiments, the catalytically inactive Cas9 molecule has reduced activity and is nCas9. In some embodiments, the Cas9 molecule comprises a nCas9 fused to one or more uracil glycosylase inhibitor (UGI) domains. In some embodiments, the Cas9 molecule comprises a nCas9 fused to an adenine base editor (ABE), for example an ABE evolved from the RNA adenine deaminase TadA. In some embodiments, the Cas9 molecule comprises a nCas9 fused to cytidine deaminase enzyme (e.g., APOBEC deaminase, pmCDA1, activation-induced cytidine deaminase (AID)).

Examples of base editors include, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa(KKH)-BE3, Target-AID, Target-AID-NG, xBE3, eA3A-BE3, BE-PLUS, TAM, CRISPR-X, ABE7.9, ABE7.10, ABE7.10*, xABE, ABESa, VQR-ABE, VRER-ABE, Sa(KKH)-ABE, and CRISPR-SKIP. Additional examples of base editors can be found, for example, in US Publication No. 2018/0312825A1, US Publication No. 2018/0312828A1, and PCT Publication No. WO 2018/165629A1, which are incorporated by reference herein in their entireties.

In some embodiments, the base editor has been further modified to inhibit base excision repair at the target site and induce cellular mismatch repair. Any of the Cas9 molecules described herein may be fused to a Gam domain (bacteriophage Mu protein) to protect the Cas9 molecule from degradation and exonuclease activity. See, e.g., Eid et al. Biochem. J. (2018) 475(11): 1955-1964.

In some embodiments, the Cas9 molecule belongs to class 2 type V of Cas endonuclease. Class 2 type V Cas endonucleases can be further categorized as type V-A, type V-B, type V-C, and type V-U. See, e.g., Stella et al. Nature Structural & Molecular Biology (2017). In some embodiments, the Cas molecule is a type V-A Cas endonuclease, such as a Cpf1 nuclease. In some embodiments, the Cas9 molecule is a type V-B Cas endonuclease, such as a C2c1 endonuclease. See, e.g., Shmakov et al. Mol Cell (2015) 60: 385-397. In some embodiments, the Cas molecule is Mad7™ (from Inscripta). Alternatively or in addition, the Cas9 molecule is a Cpf1 nuclease or a variant thereof. As will be appreciated by one of skill in the art, the Cpf1 nuclease may also be referred to as Cas12a. See, e.g., Strohkendl et al. Mol. Cell (2018) 71: 1-9. In some embodiments, a composition or method described herein involves, or a host cell expresses, a Cpf1 nuclease derived from Provetella spp. or Francisella spp., Acidaminococcus sp. (AsCpf1), Lachnospiraceae bacterium (LpCpf1), or Eubacterium rectale. In some embodiments, the nucleotide sequence encoding the Cpf1 nuclease may be codon optimized for expression in a host cell. In some embodiments, the nucleotide sequence encoding the Cpf1 endonuclease is further modified to alter the activity of the protein.

In some embodiments, catalytically inactive variants of Cas molecules (e.g., of Cas9 or Cas12a) are used according to the methods described herein. A catalytically inactive variant of Cpf1 (Cas12a) may be referred to dCas12a. As described herein, catalytically inactive variants of Cpf1 maybe fused to a function domain to form a base editor. See, e.g., Rees et al. Nature Reviews Genetics (2018) 19:770-788. In some embodiments, the catalytically inactive Cas9 molecule is dCas9. In some embodiments, the endonuclease comprises a dCas12a fused to one or more uracil glycosylase inhibitor (UGI) domains. In some embodiments, the Cas9 molecule comprises a dCas12a fused to an adenine base editor (ABE), for example an ABE evolved from the RNA adenine deaminase TadA. In some embodiments, the Cas molecule comprises a dCas12a fused to cytidine deaminase enzyme (e.g., APOBEC deaminase, pmCDA1, activation-induced cytidine deaminase (AID)).

Alternatively or in addition, the Cas9 molecule may be a Cas14 endonuclease or variant thereof. Cas14 endonucleases are derived from archaea and tend to be smaller in size (e.g., 400-700 amino acids). Additionally, Cas14 endonucleases do not require a PAM sequence. See, e.g., Harrington et al. Science (2018) 362 (6416).

Any of the Cas9 molecules described herein may be modulated to regulate levels of expression and/or activity of the Cas9 molecule at a desired time. For example, it may be advantageous to increase levels of expression and/or activity of the Cas9 molecule during particular phase(s) of the cell cycle. It has been demonstrated that levels of homology-directed repair are reduced during the G1 phase of the cell cycle, therefore increasing levels of expression and/or activity of the Cas9 molecule during the S phase, G2 phase, and/or M phase may increase homology-directed repair following the Cas endonuclease editing. In some embodiments, levels of expression and/or activity of the Cas9 molecule are increased during the S phase, G2 phase, and/or M phase of the cell cycle. In one example, the Cas9 molecule fused to the N-terminal region of human Geminin. See, e.g., Gutschner et al. Cell Rep. (2016) 14(6): 1555-1566. In some embodiments, levels of expression and/or activity of the Cas9 molecule are reduced during the G1 phase. In one example, the Cas9 molecule is modified such that it has reduced activity during the G1 phase. See, e.g., Lomova et al. Stem Cells (2018) 37(2): 284-294.

Alternatively or in addition, any of the Cas9 molecules described herein may be fused to an epigenetic modifier (e.g., a chromatin-modifying enzyme, e.g., DNA methylase, histone deacetylase). See, e.g., Kungulovski et al. Trends Genet. (2016) 32(2):101-113. Cas9 molecule fused to an epigenetic modifier may be referred to as “epieffectors” and may allow for temporal and/or transient endonuclease activity. In some embodiments, the Cas9 molecule is a dCas9 fused to a chromatin-modifying enzyme.

### Zinc Finger Nucleases

In some embodiments, a cell or cell population described herein is produced using zinc finger (ZFN) technology. In some embodiments, the ZFN recognizes a target domain described herein, e.g., in Table 1. In general, zinc finger mediated genomic editing involves use of a zinc finger nuclease, which typically comprises a zinc finger DNA binding domain and a nuclease domain. The zinc finger binding domain may be engineered to recognize and bind to any target domain of interest, e.g., may be designed to recognize a DNA sequence ranging from about 3 nucleotides to about 21 nucleotides in length, or from about 8 to about 19 nucleotides in length. Zinc finger binding domains typically comprise at least three zinc finger recognition regions (e.g., zinc fingers).

Restriction endonucleases (restriction enzymes) capable of sequence-specific binding to DNA (at a recognition site) and cleaving DNA at or near the site of binding are known in the art and may be used to form ZFN for use in genomic editing. For example, Type IIS restriction endonucleases cleave DNA at sites removed from the recognition site and have separable binding and cleavage domains. In one example, the DNA cleavage domain may be derived from the FokI endonuclease.

### TALENs

In some embodiments, a cell or cell population described herein is produced using TALEN technology. In some embodiments, the TALEN recognizes a target domain described herein, e.g., in Table 1. In general, TALENs are engineered restriction enzymes that can specifically bind and cleave a desired target DNA molecule. A TALEN typically contains a Transcriptional Activator-Like Effector (TALE) DNA-binding domain fused to a DNA cleavage domain. The DNA binding domain may contain a highly conserved 33-34 amino acid sequence with a divergent 2 amino acid RVD (repeat variable dipeptide motif) at positions 12 and 13. The RVD motif determines binding specificity to a nucleic acid sequence and can be engineered to specifically bind a desired DNA sequence. In one example, the DNA cleavage domain may be derived from the FokI endonuclease. In some embodiments, the FokI domain functions as a dimer, using two constructs with unique DNA binding domains for sites in the target genome with proper orientation and spacing.

A TALEN specific to a target gene of interest can be used inside a cell to produce a double-stranded break (DSB). A mutation can be introduced at the break site if the repair mechanisms improperly repair the break via non-homologous end joining. For example, improper repair may introduce a frame shift mutation. Alternatively, a foreign DNA molecule having a desired sequence can be introduced into the cell along with the TALEN. Depending on the sequence of the foreign DNA and chromosomal sequence, this process can be used to correct a defect or introduce a DNA fragment into a target gene of interest, or introduce such a defect into the endogenous gene, thus decreasing expression of the target gene.

Some exemplary, non-limiting embodiments of endonucleases and nuclease variants suitable for use in connection with the guide RNAs and genetic engineering methods provided herein have been described above. Additional suitable nucleases and nuclease variants will be apparent to those of skill in the art based on the present disclosure and the knowledge in the art. The disclosure is not limited in this respect.

gRNA Sequences and Configurations

A gRNA can comprise a number of domains. In an embodiment, a unimolecular, sgRNA, or chimeric, gRNA comprises, e.g., from 5′ to 3′: a targeting domain (which is complementary to a target nucleic acid in the CD33 gene; a first complementarity domain; a linking domain; a second complementarity domain (which is complementary to the first complementarity domain); a proximal domain; and optionally, a tail domain. Each of these domains is now described in more detail.

The targeting domain may comprise a nucleotide sequence that is complementary, e.g., at least 80, 85, 90, or 95% complementary, e.g., fully complementary, to the target sequence on the target nucleic acid. The targeting domain is part of an RNA molecule and will therefore comprise the base uracil (U), while any DNA encoding the gRNA molecule will comprise the base thymine (T). While not wishing to be bound by theory, in an embodiment, it is believed that the complementarity of the targeting domain with the target sequence contributes to specificity of the interaction of the gRNA/Cas9 molecule complex with a target nucleic acid. It is understood that in a targeting domain and target sequence pair, the uracil bases in the targeting domain will pair with the adenine bases in the target sequence. In an embodiment, the target domain itself comprises in the 5′ to 3′ direction, an optional secondary domain, and a core domain. In an embodiment, the core domain is fully complementary with the target sequence. In an embodiment, the targeting domain is 5 to 50 nucleotides in length. The targeting domain may be between 15-25 nucleotides, 18-22 nucleotides, or 19-21 nucleotides in length. In some embodiments, the targeting domain is 16, 17, 18, 19, 20, 21, 22, 23, 24, or 25 nucleotides in length. In some embodiments, the targeting domain is between 10-30, or between 15-25, nucleotides in length.

In some embodiments, a targeting domain comprises a core domain and a secondary targeting domain, e.g., as described in PCT Publication No. WO 2015/157070, which is incorporated by reference in its entirety. In an embodiment, the core domain comprises about 8 to about 13 nucleotides from the 3′ end of the targeting domain (e.g., the most 3′ 8 to 13 nucleotides of the targeting domain). In an embodiment, the secondary domain is positioned to the core domain. In many embodiments, the core domain has exact complementarity with the corresponding region of the target sequence. In other embodiments, the core domain can have 1 or more nucleotides that are not complementary with the corresponding nucleotide of the target sequence.

The first complementarity domain is complementary with the second complementarity domain, and in an embodiment, has sufficient complementarity to the second complementarity domain to form a duplexed region under at least some physiological conditions. In an embodiment, the first complementarity domain is 5 to 30 nucleotides in length. In an embodiment, the first complementarity domain comprises 3 subdomains, which, in the 5′ to 3′ direction are: a 5′ subdomain, a central subdomain, and a 3′ subdomain. In an embodiment, the 5′ subdomain is 4 to 9, e.g., 4, 5, 6, 7, 8 or 9 nucleotides in length. In an embodiment, the central subdomain is 1, 2, or 3, e.g., 1, nucleotide in length. In an embodiment, the 3′ subdomain is 3 to 25, e.g., 4 to 22, 4 to 18, or 4 to 10, or 3, 4, 5, 6, 7, 8, 9, 11, 12, 13, 14, 15, 16, 17, 18, 19, 20, 21, 22, 23, 24, or 25 nucleotides in length. The first complementarity domain can share homology with, or be derived from, a naturally occurring first complementarity domain. In an embodiment, it has at least 50% homology with a S. pyogenes, S. aureus or S. thermophilus, first complementarity domain.

The sequence and placement of the above-mentioned domains are described in more detail in PCT Publication No. WO 2015/157070, which is herein incorporated by reference in its entirety, including p. 88-112 therein.

A linking domain serves to link the first complementarity domain with the second complementarity domain of a unimolecular gRNA. The linking domain can link the first and second complementarity domains covalently or non-covalently. In an embodiment, the linkage is covalent. In an embodiment, the linking domain is, or comprises, a covalent bond interposed between the first complementarity domain and the second complementarity domain. In some embodiments, the linking domain comprises one or more, e.g., 2, 3, 4, 5, 6, 7, 8, 9, or 10 nucleotides. In some embodiments, the linking domain comprises at least one non-nucleotide bond, e.g., as disclosed in PCT Publication No. WO 2018/126176, the entire contents of which are incorporated herein by reference.

The second complementarity domain is complementary, at least in part, with the first complementarity domain, and in an embodiment, has sufficient complementarity to the second complementarity domain to form a duplexed region under at least some physiological conditions. In an embodiment, the second complementarity domain can include a sequence that lacks complementarity with the first complementarity domain, e.g., a sequence that loops out from the duplexed region. In an embodiment, the second complementarity domain is 5 to 27 nucleotides in length. In an embodiment, the second complementarity domain is longer than the first complementarity region. In an embodiment, the complementary domain is 5, 6, 7, 8, 9, 10, 11, 12, 13, 14, 15, 16, 17, 18, 19, 20, 21, 22, 23, 24 or 25 nucleotides in length. In an embodiment, the second complementarity domain comprises 3 subdomains, which, in the 5′ to 3′ direction are: a 5′ subdomain, a central subdomain, and a 3′ subdomain. In an embodiment, the 5′ subdomain is 3 to 25, e.g., 4 to 22, 4 to 18, or 4 to 10, or 3, 4, 5, 6, 7, 8, 9, 11, 12, 13, 14, 15, 16, 17, 18, 19, 20, 21, 22, 23, 24, or 25 nucleotides in length. In an embodiment, the central subdomain is 1, 2, 3, 4 or 5, e.g., 3, nucleotides in length. In an embodiment, the 3′ subdomain is 4 to 9, e.g., 4, 5, 6, 7, 8 or 9 nucleotides in length. In an embodiment, the 5′ subdomain and the 3′ subdomain of the first complementarity domain, are respectively, complementary, e.g., fully complementary, with the 3′ subdomain and the 5′ subdomain of the second complementarity domain.

In an embodiment, the proximal domain is 5 to 20 nucleotides in length. In an embodiment, the proximal domain can share homology with or be derived from a naturally occurring proximal domain. In an embodiment, it has at least 50% homology with a S. pyogenes, S. aureus or S. thermophilus, proximal domain.

A broad spectrum of tail domains are suitable for use in gRNAs. In an embodiment, the tail domain is 0 (absent), 1, 2, 3, 4, 5, 6, 7, 8, 9, or 10 nucleotides in length. In some embodiments, the tail domain nucleotides are from or share homology with a sequence from the 5′ end of a naturally occurring tail domain. In an embodiment, the tail domain includes sequences that are complementary to each other and which, under at least some physiological conditions, form a duplexed region. In an embodiment, the tail domain is absent or is 1 to 50 nucleotides in length. In an embodiment, the tail domain can share homology with or be derived from a naturally occurring proximal tail domain. In an embodiment, it has at least 50% homology with an S. pyogenes, S. aureus, or S. thermophilus, tail domain. In an embodiment, the tail domain includes nucleotides at the 3′ end that are related to the method of in vitro or in vivo transcription.

In some embodiments, modular gRNA comprises: a first strand comprising, e.g., from to 3′: a targeting domain (which is complementary to a target nucleic acid in the CD33 gene), and a first complementarity domain; and a second strand, comprising, preferably from 5′ to 3′: optionally, a 5′ extension domain, a second complementarity domain, a proximal domain, and optionally, a tail domain.

In some embodiments, the gRNA is chemically modified. For instance, the gRNA may comprise one or more modification chosen from phosphorothioate backbone modification, 2′-O-Me-modified sugars (e.g., at one or both of the 3′ and 5′ termini), 2′F-modified sugar, replacement of the ribose sugar with the bicyclic nucleotide-cEt, 3′thioPACE (MSP), or any combination thereof. Suitable gRNA modifications are described, e.g., in Randar et al. PNAS (2015) 112 (51) E7110-E7117 and Hendel et al., Nat Biotechnol. (2015) September; 33(9): 985-989, each of which is incorporated herein by reference in its entirety. In some embodiments, a gRNA described herein comprises one or more 2′-O-methyl-3′-phosphorothioate nucleotides, e.g., at least 2, 3, 4, 5, or 6 2′-O-methyl-3′-phosphorothioate nucleotides. In some embodiments, a gRNA described herein comprises modified nucleotides (e.g., 2′-O-methyl-3′-phosphorothioate nucleotides) at the three terminal positions and the 5′ end and/or at the three terminal positions and the 3′ end. In some embodiments, the gRNA may comprise one or more modified nucleotides, e.g., as described in PCT Publication Nos. WO/2017/214460, WO/2016/089433, and WO/2016/164356, which are incorporated by reference their entirety.

In some embodiments, a gRNA described herein is chemically modified. For example, the gRNA may comprise one or more 2′-O modified nucleotide, e.g., 2′-O-methyl nucleotide. In some embodiments, the gRNA comprises a 2′-O modified nucleotide, e.g., 2′-nucleotide at the 5′ end of the gRNA. In some embodiments, the gRNA comprises a 2′-O modified nucleotide, e.g., 2′-O-methyl nucleotide at the 3′ end of the gRNA. In some embodiments, the gRNA comprises a 2′-O-modified nucleotide, e.g., 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g. 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g. 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, the gRNA may comprise one or more 2′-O-modified and 3′phosphorous-modified nucleotide, e.g., a 2′-O-methyl 3′phosphorothioate nucleotide. In some embodiments, the gRNA comprises a 2′-O-modified and 3′phosphorous-modified, e.g., 2′-O-methyl 3′phosphorothioate nucleotide at the 5′ end of the gRNA. In some embodiments, the gRNA comprises a 2′-O-modified and 3′phosphorous-modified, e.g., 2′-O-methyl 3′phosphorothioate nucleotide at the 3′ end of the gRNA. In some embodiments, the gRNA comprises a 2′-O-modified and 3′phosphorous-modified, e.g., 2′-O-methyl 3′phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, the gRNA may comprise one or more 2′-O-modified and 3′-phosphorous-modified, e.g., 2′-O-methyl 3′thioPACE nucleotide. In some embodiments, the gRNA comprises a 2′-O-modified and 3′phosphorous-modified, e.g., 2′-O-methyl 3′thioPACE nucleotide at the 5′ end of the gRNA. In some embodiments, the gRNA comprises a 2′-O-modified and 3′phosphorous-modified, e.g., 2′-O-methyl 3′thioPACE nucleotide at the 3′ end of the gRNA. In some embodiments, the gRNA comprises a 2′-O-modified and 3′phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′phosphorous-modified, e.g. 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, the gRNA comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, the gRNA comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

Some exemplary, non-limiting embodiments of modifications, e.g., chemical modifications, suitable for use in connection with the guide RNAs and genetic engineering methods provided herein have been described above. Additional suitable modifications, e.g., chemical modifications, will be apparent to those of skill in the art based on the present disclosure and the knowledge in the art, including, but not limited to those described in Hendel, A. et al., Nature Biotech. (2015) Vol 33, No. 9; in PCT Publication Nos. WO/2017/214460; in WO/2016/089433; and/or in WO/2016/164356; each one of which is herein incorporated by reference in its entirety.

gRNAs Targeting CD33

The present disclosure provides a number of useful gRNAs that can target an endonuclease to human CD33. In some embodiments, the gRNA used in the methods described herein target a sequence in exon 3 of CD33. Table 1 below illustrates target domains in human endogenous CD33 that can be bound by gRNAs described herein.

TABLE 1
Exemplary Cas9 target site sequences of human
CD33 are provided, as are exemplary targeting
domain sequences useful for targeting such
sites. For each target site, the first
sequence represents the DNA target domain
sequence, the second sequence represents
the reverse complement thereof, and the
third sequence represents an exemplary
targeting domain sequence of a gRNA that
can be used to target the respective target site.

| gRNA | | |
|---|---|---|
| Name | Target Domain Sequences* | PAM |
| gRNA A | CCCCAGGACTACTCACTCCT (SEQ ID NO: 1) | CGG |
| | AGGAGTGAGTAGTCCTGGGG (SEQ ID NO: 5) | |
| | CCCCAGGACUACUCACUCCU (SEQ ID NO: 9) | |
| gRNA B | ACCGAGGAGTGAGTAGTCCT (SEQ ID NO: 2) | GGG |
| | AGGACTACTCACTCCTCGGT (SEQ ID NO: 6) | |
| | ACCGAGGAGUGAGUAGUCCU (SEQ ID NO: 10) | |
| gRNA C | GGTGGGGGCAGCTGACAACC (SEQ ID NO: 3) | AGG |
| | GGTTGTCAGCTGCCCCCACC (SEQ ID NO: 7) | |
| | GGUGGGGGCAGCUGACAACC (SEQ ID NO: 11) | |
| gRNA D | CGGTGCTCATAATCACCCCA (SEQ ID NO: 4) | CGG |
| | TGGGGTGATTATGAGCACCG (SEQ ID NO: 8) | |
| | CGGUGCUCAUAAUCACCCCA (SEQ ID NO: 12) | |
| gRNA E | CCTCACTAGACTTGACCCAC (SEQ ID NO: 13) | AGG |
| | GTGGGTCAAGTCTAGTGAGG (SEQ ID NO: 14) | |
| | CCUCACUAGACUUGACCCAC (SEQ ID NO: 15) | |

The CD33 (CCDS33084.1) cDNA sequence is provided below as SEQ ID NO: 16. Exon 3 is underlined.

(SEQ ID NO: 16)
ATGCCGCTGCTGCTACTGCTGCCCCTGCTGTGGGCAGGGGCCCTGGCTAT
GGATCCAAATTTCTGGCTGCAAGTGCAGGAGTCAGTGACGGTACAGGAGG
GTTTGTGCGTCCTCGTGCCCTGCACTTTCTTCCATCCCATACCCTACTAC
GACAAGAACTCCCCAGTTCATGGTTACTGGTTCCGGGAAGGAGCCATTAT
ATCCAGGGACTCTCCAGTGGCCACAAACAAGCTAGATCAAGAAGTACAGG
AGGAGACTCAGGGCAGATTCCGCCTCCTTGGGGATCCCAGTAGGAACAAC
TGCTCCCTGAGCATCGTAGACGCCAGGAGGAGGGATAATGGTTCATACTT
CTTTCGGATGGAGAGAGGAAGTACCAAATACAGTTACAAATCTCCCCAGC
TCTCTGTGCATGTGACAGACTTGACCCACAGGCCCAAAATCCTCATCCCT
GGCACTCTAGAACCCGGCCACTCCAAAAACCTGACCTGCTCTGTGTCCTG
GGCCTGTGAGCAGGGAACACCCCCGATCTTCTCCTGGTTGTCAGCTGCCC
CCACCTCCCTGGGCCCCAGGACTACTCACTCCTCGGTGCTCATAATCACC
CCACGGCCCCAGGACCACGGCACCAACCTGACCTGTCAGGTGAAGTTCGC
TGGAGCTGGTGTGACTACGGAGAGAACCATCCAGCTCAACGTCACCTATG
TTCCACAGAACCCAACAACTGGTATCTTTCCAGGAGATGGCTCAGGGAAA
CAAGAGACCAGAGCAGGAGTGGTTCATGGGGCCATTGGAGGAGCTGGTGT
TACAGCCCTGCTCGCTCTTTGTCTCTGCCTCATCTTCTTCATAGTGAAGA
CCCACAGGAGGAAAGCAGCCAGGACAGCAGTGGGCAGGAATGACACCCAC
CCTACCACAGGGTCAGCCTCCCCGAAACACCAGAAGAAGTCCAAGTTACA
TGGCCCCACTGAAACCTCAAGCTGTTCAGGTGCCGCCCCTACTGTGGAGA
TGGATGAGGAGCTGCATTATGCTTCCCTCAACTTTCATGGGATGAATCCT
TCCAAGGACACCTCCACCGAATACTCAGAGGTCAGGACCCAGTGA

Exon 3 of CD33 is provided separately below as SEQ ID NO: 17. Underlining indicates the regions complementary to gRNA A, gRNA B, gRNA C, gRNA D (or the reverse complement thereof). Note that the target regions for gRNA A, gRNA B, and gRNA D partially overlap.

(SEQ ID NO: 17)
ACTTGACCCACAGGCCCAAAATCCTCATCCCTGGCACTCTAGAACCCGGC
CACTCCAAAAACCTGACCTGCTCTGTGTCCTGGGCCTGTGAGCAGGGAAC
ACCCCCGATCTTCTCCTGGTTGTCAGCTGCCCCCACCTCCCTGGGCCCCA
GGACTACTCACTCCTCGGTGCTCATAATCACCCCACGGCCCCAGGACCAC
GGCACCAACCTGACCTGTCAGGTGAAGTTCGCTGGAGCTGGTGTGACTAC
GGAGAGAACCATCCAGCTCAACGTCACCT

Dual gRNA Compositions and Uses Thereof

In some embodiments, a gRNA described herein (e.g., a gRNA of Table 1) can be used in combination with a second gRNA, e.g., for directing nucleases to two sites in a genome. For instance, in some embodiments, it is desired to produce a hematopoietic cell that is deficient for CD33 and a second lineage-specific cell surface antigen, e.g., so that the cell can be resistant to two agents: an anti-CD33 agent and an agent targeting the second lineage-specific cell surface antigen. In some embodiments, it is desirable to contact a cell with two different gRNAs that target different regions of CD33, in order to make two cuts and create a deletion between the two cut sites. Accordingly, the disclosure provides various combinations of gRNAs.

In some embodiments, two or more (e.g., 2, 3, 4, or more) gRNAs described herein are admixed. In some embodiments, each gRNA is in a separate container. In some embodiments, a kit described herein (e.g., a kit comprising one or more gRNAs according to Table 1) also comprises a Cas9 molecule, or a nucleic acid encoding the Cas9 molecule.

In some embodiments, the first and second gRNAs are gRNAs according to Table 1 or variants thereof.

In some embodiments, the first gRNA is a CD33 gRNA described herein (e.g., a gRNA of Table 1 or a variant thereof) and the second gRNA targets a lineage-specific cell-surface antigen chosen from: CD5, CD6, CD7, BCMA, CD19, CD20, CD30, ROR1, B7H6, B7H3, CD23, CD38, C-type lectin like molecule-1, CS1, IL-5, L1-CAM, PSCA, PSMA, CD138, CD133, CD70, CD7, CD13, NKG2D, NKG2D ligand, CLEC12A, CD11, CD123, CD56, CD34, CD14, CD66b, CD41, CD61, CD62, CD235a, CD146, CD326, LMP2, CD22, CD52, CD10, CD3/TCR, CD79/BCR, and CD26.

In some embodiments, the first gRNA is a CD33 gRNA described herein (e.g., a gRNA according to Table 1 or a variant thereof) and the second gRNA targets a lineage-specific cell-surface antigen associated with a specific type of cancer, such as, without limitation, CD20, CD22 (Non-Hodgkin's lymphoma, B-cell lymphoma, chronic lymphocytic leukemia (CLL)), CD52 (B-cell CLL), CD33 (acute myeloid leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), RCAS1 (gynecological carcinomas, biliary adenocarcinomas and ductal adenocarcinomas of the pancreas) as well as prostate specific membrane antigen.

In some embodiments, the first gRNA is a CD33 gRNA described herein (e.g., a gRNA according to Table 1 or a variant thereof) and the second gRNA targets a lineage-specific cell-surface antigen chosen from: CD5, CD6, CD7, CD13, CD19, CD22, CD20, CD25, CD30, CD32, CD38, CD44, CD45, CD47, CD56, 96, CD117, CD123, CD135, CD174, CLL-1, BCMA, folate receptor (3, IL1RAP, MUC1, NKG2D/NKG2DL, TIM-3, or WT1.

In some embodiments, the first gRNA is a CD33 gRNA described herein (e.g., a gRNA according to Table 1 or a variant thereof) and the second gRNA targets a lineage-specific cell-surface antigen chosen from: CD1a, CD1b, CD1c, CD1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD85I, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD14, CDw145, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD272, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362 or CD363.

In some embodiments, the first gRNA is a CD33 gRNA described herein (e.g., a gRNA according to Table 1 or a variant thereof) and the second gRNA targets a lineage-specific cell-surface antigen chosen from: CD19; CD123; CD22; CD30; CD171; CS-1 (also referred to as CD2 subset 1, CRACC, SLAMF7, CD319, and 19A24); C-type lectin-like molecule-1 (CLECL1); epidermal growth factor receptor variant III (EGFRvIII); ganglioside G2 (CD2); ganglioside GD3 (aNeu5Ac(2-8)aNeu5Ac(2-3)bDGalp(1-4)bDGlep(1-1)Cer); TNF receptor family member B cell maturation (BCMA), Tn antigen ((Tn Ag) or (GalNAca-Ser/Thr)); prostate-specific membrane antigen (PSMA); Receptor tyrosine kinase-like orphan receptor 1 (ROR1); Fms-Like tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor I receptor (IGF-I receptor), carbonic anhydrase IX (CAIX), Proteasome (Prosome, Macropain) Subunit, Beta Type 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex; locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1), ETS translocation-variant gene 6, located on chromosome 12p (ETV6-AML); sperm protein 17 (SPA17); X Antigen Family, member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-1AP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAXS); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxy esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2), lymphocyte antigen 75 (LY75); Glypican-3 (GPC3); Fc receptor-like 5 (FCRL5); and immunoglobulin lambda-like polypeptide 1 (IGLL1).

In some embodiments, the first gRNA is a CD33 gRNA described herein (e.g., a gRNA according to Table 1 or a variant thereof) and the second gRNA targets a lineage-specific cell-surface antigen chosen from: CD11a, CD18, CD19, CD20, CD31, CD34, CD44, CD45, CD47, CD51, CD58, CD59, CD63, CD97, CD99, CD100, CD102, CD123, CD127, CD133, CD135, CD157, CD172b, CD217, CD300a, CD305, CD317, CD321, and CLL1.

In some embodiments, the first gRNA is a CD33 gRNA described herein (e.g., a gRNA according to Table 1 or a variant thereof) and the second gRNA targets a lineage-specific cell-surface antigen chosen from: CD123, CLL1, CD38, CD135 (FLT3), CD56 (NCAM1), CD117 (c-KIT), FRP (FOLR2), CD47, CD82, TNFRSF1B (CD120B), CD191, CD96, PTPRJ (CD148), CD70, LILRB2 (CD85D), CD25 (IL2Ralpha), CD44, CD96, NKG2D Ligand, CD45, CD7, CD15, CD19, CD20, CD22, CD37, and CD82.

In some embodiments, the first gRNA is a CD33 gRNA described herein (e.g., a gRNA according to Table 1 or a variant thereof) and the second gRNA targets a lineage-specific cell-surface antigen chosen from: CD7, CD11a, CD15, CD18, CD19, CD20, CD22, CD25, CD31, CD34, CD37, CD38, CD44, CD45, CD47, CD51, CD56, CD58, CD59, CD63, CD70, CD82, CD85D, CD96, CD97, CD99, CD100, CD102, CD117, CD120B, CD123, CD127, CD133, CD135, CD148, CD157, CD172b, CD191, CD217, CD300a, CD305, CD317, CD321, CLL1, FRP (FOLR2), or NKG2D Ligand.

In some embodiments, the first gRNA is a CD33 gRNA described herein (e.g., a gRNA according to Table 1 or a variant thereof) and the second gRNA targets CLL-1. In some embodiments, the first gRNA is a CD33 gRNA described herein (e.g., a gRNA according to Table 1 or a variant thereof) and the second gRNA targets CD123.

TABLE 2
Exemplary gRNA spacer sequences.

| | | SEQ |
|---|---|---|
| gRNA target | gRNA spacer sequence* | ID NO: |
| hCD33 | ACCTGTCAGGTGAAGTTCGC TGG | 28 |
| hCD33 | TGGCCGGGTTCTAGAGTGCC AGG | 29 |
| hCD33 | GGCCGGGTTCTAGAGTGCCA GGG | 30 |
| hCD33 | CACCGAGGAGTGAGTAGTCC TGG | 31 |
| hCD33 | TCCAGCGAACTTCACCTGAC AGG | 32 |
| CD33 (in intron 1) | GCTGTGGGGAGAGGGGTTGT | 33 |
| CD33 (in intron 1) | CTGTGGGGAGAGGGGTTGTC | 34 |
| CD33 (in intron 1) | TGGGGAAACGAGGGTCAGCT | 35 |
| CD33 (in intron 1) | GGGCCCCTGTGGGGAAACGA | 36 |
| CD33 (in intron 1) | AGGGCCCCTGTGGGGAAACG | 37 |
| CD33 (in intron 1) | GCTGACCCTCGTTTCCCCAC | 38 |
| CD33 (in intron 1) | CTGACCCTCGTTTCCCCACA | 39 |
| CD33 (in intron 1) | TGACCCTCGTTTCCCCACAG | 40 |
| CD33 (in intron 1) | CCATAGCCAGGGCCCCTGTG | 41 |
| CD33 (in intron 2) | GCATGTGACAGGTGAGGCAC | 42 |
| CD33 (in intron 2) | TGAGGCACAGGCTTCAGAAG | 43 |
| CD33 (in intron 2) | AGGCTTCAGAAGTGGCCGCA | 44 |
| CD33 (in intron 2) | GGCTTCAGAAGTGGCCGCAA | 45 |
| CD33 (in intron 2) | GTACCCATGAACTTCCCTTG | 46 |
| CD33 (in intron 2) | GTGGCCGCAAGGGAAGTTCA | 47 |
| CD33 (in intron 2) | TGGCCGCAAGGGAAGTTCAT | 48 |
| CD33 (in intron 2) | GGAAGTTCATGGGTACTGCA | 49 |
| CD33 (in intron 2) | TTCATGGGTACTGCAGGGCA | 50 |
| CD33 (in intron 2) | CTAAACCCCTCCCAGTACCA | 51 |
| CD33 (in intron 1) | CACTCACCTGCCCACAGCAG | 52 |
| CD33 (in intron 1) | CCCTGCTGTGGGCAGGTGAG | 53 |
| CD33 (in intron 1) | TGGGCAGGTGAGTGGCTGTG | 54 |
| CD33 (in intron 1) | GGTGAGTGGCTGTGGGGAGA | 55 |
| CD33 (in intron 1) | GTGAGTGGCTGTGGGGAGAG | 56 |
| CD33 (exon 2) | ATCCATAGCCAGGGCCCCTG | 64 |
| CD33 (exon 2) | TCCATAGCCAGGGCCCCTGT | 57 |
| CD33 (exon 2) | CCATAGCCAGGGCCCCTGTG | 58 |
| CD33 (exon 2) | TCGTTTCCCCACAGGGGCCC | 65 |
| CD33 (exon 2) | TGGCTATGGATCCAAATTTC | 66 |
| CD33 (exon 2) | TGGGGAAACGAGGGTCAGCT | 59 |
| CD33 (exon 2) | GGGCCCCTGTGGGGAAACGA | 60 |
| CD33 (exon 2) | AGAAATTTGGATCCATAGCC AGG | 61 |
| CD33 (exon 3) | ATCCCTGGCACTCTAGAACC CGG | 62 |
| CD33 (exon 3) | CCTCACTAGACTTGACCCAC AGG | 63 |

*Certain gRNA spacer sequences are followed by a PAM sequence,
indicated by a space in the text.

Some of the embodiments, advantages, features, and uses of the technology disclosed herein will be more fully understood from the Examples below. The Examples are intended to illustrate some of the benefits of the present disclosure and to describe particular embodiments but are not intended to exemplify the full scope of the disclosure and, accordingly, do not limit the scope of the disclosure.

### EXAMPLES

### Example 1. Generation of Genetically Engineered Hematopoietic Cells Comprising a Modified Gene Encoding CD33

The Cas9 sgRNAs indicated in Table 1 were designed based on the SpCas9 PAM (5′-NGG-3′) with close proximity to the target region and evaluated for predicted specificity by minimizing potential off-target sites in the human genome with an online search algorithm (e.g., the Benchling algorithm, Doench et al 2016, Hsu et al 2013).

Cas9 sgRNAs are synthesized using the gRNA targeting domains provided below and the Cas9 sgRNA scaffold sequence 5′-GUUUUAGAGCUAGAAAUAGCAAGUUAAAAUAAGGCUAGUCCGUUAUCAAC UUGAAAAAGUGGCACCGAGUCGGUGCUUUU-3′ (SEQ ID NO: 18).

For example, the nucleotide sequence of sgRNA A is 5′-CCCCAGGACUACUCACUCCUGUUUUAGAGCUAGAAAUAGCAAGUUAAAAUA AGGCUAGUCCGUUAUCAACUUGAAAAAGUGGCACCGAGUCGGUGCUUUU-3′ (SEQ ID NO: 19, targeting domain sequence in bold).

For example, the nucleotide sequence of sgRNA B is 5′-ACCGAGGAGUGAGUAGUCCUGUUUUAGAGCUAGAAAUAGCAAGUUAAAAUA AGGCUAGUCCGUUAUCAACUUGAAAAAGUGGCACCGAGUCGGUGCUUUU-3′ (SEQ ID NO: 20, targeting domain sequence in bold).

For example, the nucleotide sequence of sgRNA C is 5′-GGUGGGGGCAGCUGACAACCGUUUUAGAGCUAGAAAUAGCAAGUUAAAAUA AGGCUAGUCCGUUAUCAACUUGAAAAAGUGGCACCGAGUCGGUGCUUUU-3′ (SEQ ID NO: 21, targeting domain sequence in bold).

For example, the nucleotide sequence of sgRNA D is 5′-CGGUGCUCAUAAUCACCCCAGUUUUAGAGCUAGAAAUAGCAAGUUAAAAUA AGGCUAGUCCGUUAUCAACUUGAAAAAGUGGCACCGAGUCGGUGCUUUU-3′ (SEQ ID NO: 22, targeting domain sequence in bold).

For example, the nucleotide sequence of sgRNA E is 5′-CCUCACUAGACUUGACCCACGUUUUAGAGCUAGAAAUAGCAAGUUAAAAUA AGGCUAGUCCGUUAUCAACUUGAAAAAGUGGCACCGAGUCGGUGCUUUU-3′ (SEQ ID NO: 23, targeting domain sequence in bold).

All designed synthetic sgRNAs are produced with chemically modified nucleotides at the three terminal positions at both the 5′ and 3′ ends. The modified nucleotides contained 2′-O-methyl-3′-phosphorothioate (abbreviated as “ms”) and the ms-sgRNAs are HPLC-purified. Cas9 protein is purchased from Synthego.

For example, the nucleotide sequence of sgRNA A, showing the modified nucleotides, is 5′-CmsCmsCmsCAGGACUACUCACUCCUGUUUUAGAGCUAGAAAUAGCAAGUUAA AAUAAGGCUAGUCCGUUAUCAACUUGAAAAAGUGGCACCGAGUCGGUGCUmsU msUmsU-3′ (SEQ ID NO: 24, targeting domain sequence in bold).

For example, the nucleotide sequence of sgRNA E, showing the modified nucleotides, is 5′-CmsCmssUmsCACUAGACUUGACCCACGUUUUAGAGCUAGAAAUAGCAAGUUAA AAUAAGGCUAGUCCGUUAUCAACUUGAAAAAGUGGCACCGAGUCGGUGCUmsU msUmsU-3′ (SEQ ID NO: 25, targeting domain sequence in bold).

Peripheral blood mononuclear cells are collected from healthy donor subject by apheresis following hematopoietic stem cell mobilization. The donor CD34+ cells are electroporated with Cas9 protein and any of the indicated CD33-targeting Cas9 gRNAs disclosed herein, e.g., having the targeting domain sequences provided in Tables 1 and 3, e.g., gRNA A, gRNA, B, gRNA C, gRNA D, or gRNA E.

| gRNA | | |
|---|---|---|
| Name | Targeting Domain Sequence | PAM |
| gRNA A | CCCCAGGACUACUCACUCCU (SEQ ID NO: 9) | CGG |
| gRNA B | ACCGAGGAGUGAGUAGUCCU (SEQ ID NO: 10) | GGG |
| gRNA C | GGUGGGGGCAGCUGACAACC (SEQ ID NO: 11) | AGG |
| gRNA D | CGGUGCUCAUAAUCACCCCA (SEQ ID NO: 12) | CGG |
| gRNA E | CCUCACUAGACUUGACCCAC (SEQ ID NO: 15) | AGG |

The edited cells are cultured for less than 48 hours. Upon harvest, the cells are washed, resuspended in the final formulation, and cryopreserved.

A representative sample of the edited HSCs is evaluated for viability and expression of CD33, or absence thereof, by staining for CD33 using an anti-CD33 antibody (e.g., P67.7) and analyzed by flow cytometry. Edited CD33KO eHSPC populations exhibiting at least 70% cell viability and at least 45% CD33 editing efficiency (i.e., absence of CD33 expression in at least 45% of the cells in the cell population) at 48 hours after electroporation are used for HCT.

### Example 2: Combination Treatment with CD33KO eHSPC and CD33-Targeted ADC (Mylotarg)

CD33 is a transmembrane receptor that is expressed on normal myeloid cells, as well as most leukemic myeloblasts (e.g., Larson et al. Cancer (2005) 104(7): 1442-1452; Kenderian et al. Leukemia (2015) 29(8): 1637-47; Wang et al. Mol. Ther. (2015) 23(1): 184-91; Pollard et al. J. Clin. Oncol. (2016) 34(7: 747-55). Hematopoietic stem cells that are genetically engineered to have reduced or eliminated expression of CD33 (“CD33KO eHSCs” or “CD33KO eHSPCs”) have the potential to improve the safety and efficacy of CD33 directed therapies, as they are not susceptible to the on-target, off-cancer cytotoxicity reported to be associated with CD33 directed therapies, and thus enable administration of the CD33 directed therapies at an optimal dose and schedule, e.g., without treatment delays or dose omissions.

The treatment regimen provided herein is directed to subjects having acute myeloid leukemia, or a pre-malignant stage thereof, e.g., myelodysplastic syndrome. Currently, CD33-directed therapies are limited by on-target cytotoxicity directed toward normal myeloid lineage cells. The approach provided herein eliminates this on-target toxicity by administering genetically engineered HSPC that lack expression of the CD33 epitope recognized by the CD33 targeted therapy. Subsequently, the normal myeloid compartment is protected from the on-target effects of CD33 targeted therapy leading to an improved therapeutic index for these agents and potentially better outcomes for subjects with AML.

This example provides a treatment regimen using allogeneic or autologous CRISPR/Cas9 genome-edited CD33KO eHSPCs lacking expression of CD33. Allogeneic eHSPCs are obtained by processing CD34-positive (CD34+) enriched stem cells obtained from a healthy donor who is HLA matched to recipient, i.e., the subject receiving the CD33KO eHSPCs. Autologous eHSPCs are obtained by processing CD34-positive (CD34+) enriched stem cells obtained from the same subject being treated, i.e., the HSPC donor and the subject receiving the CD33KO eHSPCs are the same. The CD33KO eHSPCs are infused into the recipient subject after receiving a conditioning regimen as part of a hematopoietic cell transplant (HCT).

### Gemtuzumab Ozogamicin

Gemtuzumab ozogamicin/Mylotarg® is a CD33-directed antibody drug conjugate (ADC) approved by the U.S. Food and Drug Administration (FDA) to treat both newly diagnosed CD33-positive (CD33+) adult subjects with AML, as well as subjects with relapsed or refractory AML (R/R AML) who are 1 month of age and older.

In the case of relapsed or refractory subjects with AML, gemtuzumab ozogamicin/Mylotarg® is currently the only CD33-directed therapy approved by the U.S. FDA. Analyses provided in the “Highlights of Prescribing Information” of the U.S. Prescribing Information for Mylotarg® (Mylotarg 2020), as well as in the U.S. FDA publication on the approval summary, suggest that at doses of 2 mg/m² (Norsworthy 2018), available CD33 is saturated in subjects with AML. In addition, the risk for sinusoidal obstruction syndrome/veno-occlusive disease (SOS/VOD), which is a severe and sometimes lethal toxicity associated with gemtuzumab ozogamicin/Mylotarg® administration, is substantially reduced at lower doses. This potentially larger margin of safety supports using a reduced dose in the post-HCT setting where subjects are known to be at higher risk for SOS/VOD. In the current gemtuzumab ozogamicin/Mylotarg® U.S. FDA approved product label, a “continuation” dose and schedule of 2 mg/m² administered on day 1 of every 4 week treatment cycle for up to 8 cycles is listed for subjects with AML who are without evidence of disease progression.

In the clinical treatment regimens provided herein, a similar “continuation” dose and schedule is used as soon as 60 days post-HCT in subjects who have received CD33KO eHSPCs as part of their HCT. This provides for a potentially larger margin of safety in administering gemtuzumab ozogamicin/Mylotarg® in the post-HCT setting, to suppress early leukemia relapse, with the rationale that this allows the subject to undergo more robust immunological reconstitution, which provides a longer term “graft versus leukemia” effect.

### Subjects

The clinical regimens for treating subjects having AML, or a pre-malignant form thereof, with a stem cell transplant comprising CD33KO eHSPCs and the ADC gemtuzumab ozogamicin/Mylotarg® as provided herein are useful for treating subjects having, or diagnosed with, AML that is characterized by expression of CD33, or a pre-malignant form of AML, e.g., MDS, that is characterized by expression of CD33. This includes subjects that are naïve to AML therapy; subjects who have received some form of AML therapy, including, for example, induction therapy; subjects who have experienced a complete hematological remission (CR1 or CR2, including complete remission with incomplete recovery of peripheral counts [CRi]) in response to AML therapy; and subjects with persistent or progressive disease, including, for example, subjects with persistent disease, which includes, for example, subjects with bone marrow blast counts of ≤10% and no clinical evidence of circulating blasts. The regimens provided herein are also useful for treating subjects having myelodysplastic syndrome (MDS) characterized by expression of CD33, including, for example, subjects who are at high risk of progression from MDS to AML.

The clinical regimens for treating subjects having AML, or a pre-malignant form thereof, with a hematopoietic stem cell transplant comprising CD33KO eHSPCs and the ADC gemtuzumab ozogamicin/Mylotarg® as provided herein regimen are further useful for treating subjects that exhibit one or more of the following adverse risk features: 1) intermediate or high-risk disease-related genetics at presentation and bone marrow that harbors evidence of MRD+ at the time of HCT; or 2) persistence of bone marrow-only leukemic blasts (≤10%) at the time of HCT (with any risk category of disease-related genetics at presentation).

### Pre-HCT Conditioning

Typically, a clinical treatment regimen for including a hematopoietic stem cell transplant comprising CD33KO eHSPCs, including, for example, an HLA-matched allogeneic HCT, as provided in some of the examples herein, comprises a full conditioning regimen. The conditioning regimen may include, for example, busulfan/melphalan/fludarabine/rabbit anti-thymocyte globulin (rATG); or total body irradiation/cyclophosphamide/thiotepa/rATG. The appropriate conditioning regimen is selected for a given subject according to clinical guidelines, taking into account the subject's health and medical history.

### Clinical Monitoring

Subjects receiving a clinical regimen including a hematopoietic stem cell transplant comprising CD33KO eHSPCs and the ADC gemtuzumab ozogamicin/Mylotarg®, as provided herein, are monitored for treatment-related adverse effects during the course of the treatment regimen, as well as during any conditioning or induction regimens that may be indicated, and are also assessed for disease status and the status of HCT graft and the hematopoietic system during treatment and/or after completion of the treatment regimen.

### Example 3: Treatment of a Subject Having AML with CD33KO eHSPC Generated Using gRNA a, and the CD33-Targeted ADC Gemtuzumab Ozogamicin/Mylotarg®

A subject having CD33-positive AML is treated with an allogeneic HCT comprising CD33KO eHSPCs and with the ADC gemtuzumab ozogamicin/Mylotarg®.

For the HCT, a population of cells comprising CD34+ hematopoietic stem cells is obtained from a healthy donor who is HLA matched at 8/8 loci (HLA-A, —B, —C, DRB1) to the subject.

After G-CSF/plerixafor mobilization, up to two apheresis procedures are performed in order to obtain a minimum of 10×10⁶ viable cells/kg (where kg refers to recipient subject weight) from the donor for processing and subsequent administration to the recipient subject. From this apheresis product, at least 3.0×10⁶ viable cells/kg (recipient weight) undergo minimal manipulation and are cryopreserved to serve as a back-up stem cell source, e.g., for use as a rescue dose. The remainder of the apheresis product is used for processing and preparation of the CD33KO eHSPC population for HCT. The CD33KO eHSPC population for HCT is prepared by enriching the apheresis product for CD34+ cells, followed by electroporation and editing with a CD33 gRNA/Cas9 complex, as described in Example 1, using a Cas9 sgRNA comprising the nucleotide sequence 5′-CCCCAGGACUACUCACUCCUGUUUUAGAGCUAGAAAUAGCAAGUUAAAAUA AGGCUAGUCCGUUAUCAACUUGAAAAAGUGGCACCGAGUCGGUGCUUUU-3′ (SEQ ID NO: 19, targeting domain sequence in bold, chemical modifications as described in Example 1).

The edited cells are subsequently placed in culture for <48 hours. Upon harvest, after the culture duration is finished, cells are washed, resuspended in the final formulation, and cryopreserved. Cell viability and editing efficiency are confirmed using a representative sample as described in Example 1, and CD33KO eHSPC populations meeting the criteria set forth in Example 1 (at least 70% viability and at least 45% CD33 editing efficiency) are used for HCT. A population for administration to a subject comprises a CD33KO eHSPC population satisfying these viability and editing efficiency criteria of at least 3×10⁶ cells/kg body weight of the recipient subject, and preferably comprises at least 4×10⁶ cells/kg, 5×10⁶ cells/kg, 6×10⁶ cells/kg, or 7×10⁶ cells/kg of the recipient subject.

After completion of the conditioning regimen, the subject receives an HCT comprising the thawed CD33KO eHSPCs via an intravenous (IV) infusion. The day of the HCT is day 0 of the treatment regimen.

The subject is assessed for CD33KO eHSPC engraftment at day 28 by measuring the absolute peripheral neutrophil count (ANC) for CD33KO (CD33−) neutrophils in the subject. The subject is deemed to exhibit neutrophil recovery (also referred to as successful CD33KO neutrophil engraftment) if the subject exhibits an absolute peripheral CD33KO neutrophil count of ≥1000/dL CD33− ANC at 28 days after CD33KO eHSPC HCT.

If the subject exhibits neutrophil recovery at day 28, a bone marrow biopsy is obtained from the subject on day 60 in order to assess disease status and hematopoietic recovery. In addition, percent donor chimerism and CD33-negative (CD33−) myeloid hematopoiesis are determined from the peripheral blood at this time. If the subject exhibits successful CD33− HSC engraftment and CD33− hematopoiesis at day 60, the subject is subsequently administered gemtuzumab ozogamicin/Mylotarg®. The CD33− ANC is monitored in the subject prior to administration of gemtuzumab ozogamicin/Mylotarg®, and the subject should preferably have ≥1000/dL CD33− ANC prior to receiving gemtuzumab ozogamicin/Mylotarg®.

Administration of gemtuzumab ozogamicin/Mylotarg® is preferably initiated within 30 days of the bone marrow biopsy at day 60, i.e., is preferably initiated by day 90. However, initiation of gemtuzumab ozogamicin/Mylotarg® may be delayed up to day 120 if a subject's clinical status, e.g., in view of comorbidities, including, for example, HCT-related comorbidities, necessitate such a delay, or in order to allow attainment of ≥1000/dL CD33-ANC in a subject. If gemtuzumab ozogamicin/Mylotarg® is initiated more than 30 days after the Day 60 bone marrow biopsy, a repeat bone marrow biopsy is completed prior to starting gemtuzumab ozogamicin/Mylotarg®.

Gemtuzumab ozogamicin/Mylotarg® is administered to the subject at a dose within the range of 0.1 mg/m² to 2 mg/m², e.g., at a dose of 0.1 mg/m², 0.25 mg/m², 0.5 mg/m², 1 mg/m², or 2 mg/m². A dose of 2 mg/m² of gemtuzumab ozogamicin/Mylotarg® is preferred for most subjects. However, some subjects may be administered a lower dose, e.g., in the event of treatment-related adverse effects, e.g., dose-limited toxicities (DLT), or in view of the health status, comorbidities, or the medical history of the individual subject.

Gemtuzumab ozogamicin/Mylotarg® is administered to the subject in a regimen of 4-week (28d) treatment cycles, wherein the subject receives the entire dose of a respective treatment cycle, e.g., at 2 mg/m² of gemtuzumab ozogamicin/Mylotarg®, on day 1 of the respective 4-week (28d) treatment cycle.

For most subjects, four consecutive treatment cycles of gemtuzumab ozogamicin/Mylotarg®, and thus four doses of gemtuzumab ozogamicin/Mylotarg®, spread 4 weeks apart from each other, are preferred. However, in some subjects, additional gemtuzumab ozogamicin/Mylotarg® treatment cycles may be indicated, e.g., up to four additional “continuation” treatment cycles, e.g., at the same dose of the initial four treatment cycles, or at a lower dose, if clinically advisable, e.g., based on the subject's clinical status.

At completion of the last gemtuzumab ozogamicin/Mylotarg® treatment cycle, the subject is monitored for disease status and hematopoietic chimerism and is monitored for these parameters every six months for five years after completion of the final treatment cycle.

### Example 4: Treatment of a Subject Having AML with CD33KO eHSPC Generated Using gRNA B, and the CD33-Targeted ADC Gemtuzumab Ozogamicin/Mylotarg®

A subject having CD33-positive AML is treated with an allogeneic HCT comprising CD33KO eHSPCs and with the ADC gemtuzumab ozogamicin/Mylotarg®.

For the HCT, a population of cells comprising CD34+ hematopoietic stem cells is obtained from a healthy donor who is HLA matched at 8/8 loci (HLA-A, —B, —C, DRB1) to the subject.

After G-CSF/plerixafor mobilization, up to two apheresis procedures are performed in order to obtain a minimum of 10×10⁶ viable cells/kg (where kg refers to recipient subject weight) from the donor for processing and subsequent administration to the recipient subject. From this apheresis product, at least 3.0×10⁶ viable cells/kg (recipient weight) undergo minimal manipulation and are cryopreserved to serve as a back-up stem cell source, e.g., for use as a rescue dose. The remainder of the apheresis product is used for processing and preparation of the CD33KO eHSPC population for HCT.

The CD33KO eHSPC population for HCT is prepared by enriching the apheresis product for CD34+ cells, followed by electroporation and editing with a CD33 gRNA/Cas9 complex, as described in Example 1, using a Cas9 sgRNA comprising the nucleotide sequence 5′-ACCGAGGAGUGAGUAGUCCUGUUUUAGAGCUAGAAAUAGCAAGUUAAAAU AAGGCUAGUCCGUUAUCAACUUGAAAAAGUGGCACCGAGUCGGUGCUUUU-3′ (SEQ ID NO: 20, targeting domain sequence in bold, chemical modifications as described for gRNAs in Example 1).

The edited cells are subsequently placed in culture for <48 hours. Upon harvest, after the culture duration is finished, cells are washed, resuspended in the final formulation, and cryopreserved. Cell viability and editing efficiency are confirmed using a representative sample as described in Example 1, and CD33KO eHSPC populations meeting the criteria set forth in Example 1 (at least 70% viability and at least 45% CD33 editing efficiency) are used for HCT. A population for administration to a subject comprises a CD33KO eHSPC population satisfying these viability and editing efficiency criteria of at least 3×10⁶ cells/kg body weight of the recipient subject, and preferably comprises at least 4×10⁶ cells/kg, 5×10⁶ cells/kg, 6×10⁶ cells/kg, or 7×10⁶ cells/kg of the recipient subject.

After completion of the conditioning regimen, the subject receives an HCT comprising the thawed CD33KO eHSPCs via an intravenous (IV) infusion. The day of the HCT is day 0 of the treatment regimen.

The subject is assessed for CD33KO eHSPC engraftment at day 28 by measuring the absolute peripheral neutrophil count (ANC) for CD33KO (CD33−) neutrophils in the subject. The subject is deemed to exhibit neutrophil recovery (also referred to as successful CD33KO neutrophil engraftment) if the subject exhibits an absolute peripheral CD33KO neutrophil count of ≥1000/dL CD33− ANC at 28 days after CD33KO eHSPC HCT.

If the subject exhibits neutrophil recovery at day 28, a bone marrow biopsy is obtained from the subject on day 60 in order to assess disease status and hematopoietic recovery. In addition, percent donor chimerism and CD33-negative (CD33−) myeloid hematopoiesis are determined from the peripheral blood at this time. If the subject exhibits successful CD33-HSC engraftment and CD33-hematopoiesis at day 60, the subject is subsequently administered gemtuzumab ozogamicin/Mylotarg®. The CD33-ANC is monitored in the subject prior to administration of gemtuzumab ozogamicin/Mylotarg®, and the subject should preferably have ≥1000/dL CD33-ANC prior to receiving gemtuzumab ozogamicin/Mylotarg®.

Administration of gemtuzumab ozogamicin/Mylotarg is preferably initiated within 30 days of the bone marrow biopsy at day 60, i.e., is preferably initiated by day 90. However, initiation of gemtuzumab ozogamicin/Mylotarg® may be delayed up to day 120 if a subject's clinical status, e.g., in view of comorbidities, including, for example, HCT-related comorbidities, necessitate such a delay, or in order to allow attainment of ≥1000/dL CD33-ANC in a subject. If gemtuzumab ozogamicin/Mylotarg® is initiated more than 30 days after the Day 60 bone marrow biopsy, a repeat bone marrow biopsy is completed prior to starting gemtuzumab ozogamicin/Mylotarg®.

Gemtuzumab ozogamicin/Mylotarg® is administered to the subject at a dose within the range of 0.1 mg/m² to 2 mg/m², e.g., at a dose of 0.1 mg/m², 0.25 mg/m², 0.5 mg/m², 1 mg/m², or 2 mg/m². A dose of 2 mg/m² of gemtuzumab ozogamicin/Mylotarg® is preferred for most subjects. However, some subjects may be administered a lower dose, e.g., in the event of treatment-related adverse effects, e.g., dose-limited toxicities (DLT), or in view of the health status, comorbidities, or the medical history of the individual subject.

Gemtuzumab ozogamicin/Mylotarg® is administered to the subject in a regimen of 4-week (28d) treatment cycles, wherein the subject receives the entire dose of a respective treatment cycle, e.g., at 2 mg/m² of gemtuzumab ozogamicin/Mylotarg®, on day 1 of the respective 4-week (28 d) treatment cycle.

For most subjects, four consecutive treatment cycles of gemtuzumab ozogamicin/Mylotarg®, and thus four doses of gemtuzumab ozogamicin/Mylotarg®, spread 4 weeks apart from each other, are preferred. However, in some subjects, additional gemtuzumab ozogamicin/Mylotarg® treatment cycles may be indicated, e.g., up to four additional “continuation” treatment cycles, e.g., at the same dose of the initial four treatment cycles, or at a lower dose, if clinically advisable, e.g., based on the subject's clinical status.

At completion of the last gemtuzumab ozogamicin/Mylotarg® treatment cycle, the subject is monitored for disease status and hematopoietic chimerism and is monitored for these parameters every six months for five years after completion of the final treatment cycle.

### Example 5: Treatment of a Subject Having AML with CD33KO eHSPC Generated Using gRNA C, and the CD33-Targeted ADC Gemtuzumab Ozogamicin/Mylotarg®

A subject having CD33-positive AML is treated with an allogeneic HCT comprising CD33KO eHSPCs and with the ADC gemtuzumab ozogamicin/Mylotarg®.

For the HCT, a population of cells comprising CD34+ hematopoietic stem cells is obtained from a healthy donor who is HLA matched at 8/8 loci (HLA-A, —B, —C, DRB1) to the subject.

After G-CSF/plerixafor mobilization, up to two apheresis procedures are performed in order to obtain a minimum of 10×10⁶ viable cells/kg (where kg refers to recipient subject weight) from the donor for processing and subsequent administration to the recipient subject. From this apheresis product, at least 3.0×10⁶ viable cells/kg (recipient weight) undergo minimal manipulation and are cryopreserved to serve as a back-up stem cell source, e.g., for use as a rescue dose. The remainder of the apheresis product is used for processing and preparation of the CD33KO eHSPC population for HCT.

The CD33KO eHSPC population for HCT is prepared by enriching the apheresis product for CD34+ cells, followed by electroporation and editing with a CD33 gRNA/Cas9 complex, as described in Example 1, using a Cas9 sgRNA comprising the nucleotide sequence 5′-GGUGGGGGCAGCUGACAACCGUUUUAGAGCUAGAAAUAGCAAGUUAAAA UAAGGCUAGUCCGUUAUCAACUUGAAAAAGUGGCACCGAGUCGGUGCUUUU-3′ (SEQ ID NO: 21, targeting domain sequence in bold, chemical modifications as described for gRNAs in Example 1).

The edited cells are subsequently placed in culture for <48 hours. Upon harvest, after the culture duration is finished, cells are washed, resuspended in the final formulation, and cryopreserved. Cell viability and editing efficiency are confirmed using a representative sample as described in Example 1, and CD33KO eHSPC populations meeting the criteria set forth in Example 1 (at least 70% viability and at least 45% CD33 editing efficiency) are used for HCT. A population for administration to a subject comprises a CD33KO eHSPC population satisfying these viability and editing efficiency criteria of at least 3×10⁶ cells/kg body weight of the recipient subject, and preferably comprises at least 4×10⁶ cells/kg, 5×10⁶ cells/kg, 6×10⁶ cells/kg, or 7×10⁶ cells/kg of the recipient subject.

After completion of the conditioning regimen, the subject receives an HCT comprising the thawed CD33KO eHSPCs via an intravenous (IV) infusion. The day of the HCT is day 0 of the treatment regimen.

The subject is assessed for CD33KO eHSPC engraftment at day 28 by measuring the absolute peripheral neutrophil count (ANC) for CD33KO (CD33−) neutrophils in the subject. The subject is deemed to exhibit neutrophil recovery (also referred to as successful CD33KO neutrophil engraftment) if the subject exhibits an absolute peripheral CD33KO neutrophil count of ≥1000/dL CD33-ANC at 28 days after CD33KO eHSPC HCT.

If the subject exhibits neutrophil recovery at day 28, a bone marrow biopsy is obtained from the subject on day 60 in order to assess disease status and hematopoietic recovery. In addition, percent donor chimerism and CD33-negative (CD33−) myeloid hematopoiesis are determined from the peripheral blood at this time. If the subject exhibits successful CD33-HSC engraftment and CD33-hematopoiesis at day 60, the subject is subsequently administered gemtuzumab ozogamicin/Mylotarg®. The CD33-ANC is monitored in the subject prior to administration of gemtuzumab ozogamicin/Mylotarg®, and the subject should preferably have ≥1000/dL CD33-ANC prior to receiving gemtuzumab ozogamicin/Mylotarg®.

Administration of gemtuzumab ozogamicin/Mylotarg is preferably initiated within 30 days of the bone marrow biopsy at day 60, i.e., is preferably initiated by day 90. However, initiation of gemtuzumab ozogamicin/Mylotarg® may be delayed up to day 120 if a subject's clinical status, e.g., in view of comorbidities, including, for example, HCT-related comorbidities, necessitate such a delay, or in order to allow attainment of ≥1000/dL CD33-ANC in a subject. If gemtuzumab ozogamicin/Mylotarg® is initiated more than 30 days after the Day 60 bone marrow biopsy, a repeat bone marrow biopsy is completed prior to starting gemtuzumab ozogamicin/Mylotarg®.

Gemtuzumab ozogamicin/Mylotarg® is administered to the subject at a dose within the range of 0.1 mg/m² to 2 mg/m², e.g., at a dose of 0.1 mg/m², 0.25 mg/m², 0.5 mg/m², 1 mg/m², or 2 mg/m². A dose of 2 mg/m² of gemtuzumab ozogamicin/Mylotarg® is preferred for most subjects. However, some subjects may be administered a lower dose, e.g., in the event of treatment-related adverse effects, e.g., dose-limited toxicities (DLT), or in view of the health status, comorbidities, or the medical history of the individual subject.

Gemtuzumab ozogamicin/Mylotarg® is administered to the subject in a regimen of 4-week (28d) treatment cycles, wherein the subject receives the entire dose of a respective treatment cycle, e.g., at 2 mg/m² of gemtuzumab ozogamicin/Mylotarg®, on day 1 of the respective 4-week (28d) treatment cycle.

For most subjects, four consecutive treatment cycles of gemtuzumab ozogamicin/Mylotarg®, and thus four doses of gemtuzumab ozogamicin/Mylotarg®, spread 4 weeks apart from each other, are preferred. However, in some subjects, additional gemtuzumab ozogamicin/Mylotarg® treatment cycles may be indicated, e.g., up to four additional “continuation” treatment cycles, e.g., at the same dose of the initial four treatment cycles, or at a lower dose, if clinically advisable, e.g., based on the subject's clinical status.

At completion of the last gemtuzumab ozogamicin/Mylotarg® treatment cycle, the subject is monitored for disease status and hematopoietic chimerism and is monitored for these parameters every six months for five years after completion of the final treatment cycle.

### Example 6: Treatment of a Subject Having AML with CD33KO eHSPC Generated Using gRNA D, and the CD33-Targeted ADC Gemtuzumab Ozogamicin/Mylotarg®

A subject having CD33-positive AML is treated with an allogeneic HCT comprising CD33KO eHSPCs and with the ADC gemtuzumab ozogamicin/Mylotarg®.

For the HCT, a population of cells comprising CD34+ hematopoietic stem cells is obtained from a healthy donor who is HLA matched at 8/8 loci (HLA-A, —B, —C, DRB1) to the subject.

After G-CSF/plerixafor mobilization, up to two apheresis procedures are performed in order to obtain a minimum of 10×10⁶ viable cells/kg (where kg refers to recipient subject weight) from the donor for processing and subsequent administration to the recipient subject. From this apheresis product, at least 3.0×10⁶ viable cells/kg (recipient weight) undergo minimal manipulation and are cryopreserved to serve as a back-up stem cell source, e.g., for use as a rescue dose. The remainder of the apheresis product is used for processing and preparation of the CD33KO eHSPC population for HCT.

The CD33KO eHSPC population for HCT is prepared by enriching the apheresis product for CD34+ cells, followed by electroporation and editing with a CD33 gRNA/Cas9 complex, as described in Example 1, using a Cas9 sgRNA comprising the nucleotide sequence 5′-CGGUGCUCAUAAUCACCCCAGUUUUAGAGCUAGAAAUAGCAAGUUAAAAU AAGGCUAGUCCGUUAUCAACUUGAAAAAGUGGCACCGAGUCGGUGCUUUU-3′ (SEQ ID NO: 22, targeting domain sequence in bold, chemical modifications as described for gRNAs in Example 1).

The edited cells are subsequently placed in culture for <48 hours. Upon harvest, after the culture duration is finished, cells are washed, resuspended in the final formulation, and cryopreserved. Cell viability and editing efficiency are confirmed using a representative sample as described in Example 1, and CD33KO eHSPC populations meeting the criteria set forth in Example 1 (at least 70% viability and at least 45% CD33 editing efficiency) are used for HCT. A population for administration to a subject comprises a CD33KO eHSPC population satisfying these viability and editing efficiency criteria of at least 3×10⁶ cells/kg body weight of the recipient subject, and preferably comprises at least 4×10⁶ cells/kg, 5×10⁶ cells/kg, 6×10⁶ cells/kg, or 7×10⁶ cells/kg of the recipient subject.

After completion of the conditioning regimen, the subject receives an HCT comprising the thawed CD33KO eHSPCs via an intravenous (IV) infusion. The day of the HCT is day 0 of the treatment regimen.

The subject is assessed for CD33KO eHSPC engraftment at day 28 by measuring the absolute peripheral neutrophil count (ANC) for CD33KO (CD33−) neutrophils in the subject. The subject is deemed to exhibit neutrophil recovery (also referred to as successful CD33KO neutrophil engraftment) if the subject exhibits an absolute peripheral CD33KO neutrophil count of ≥1000/dL CD33-ANC at 28 days after CD33KO eHSPC HCT.

If the subject exhibits neutrophil recovery at day 28, a bone marrow biopsy is obtained from the subject on day 60 in order to assess disease status and hematopoietic recovery. In addition, percent donor chimerism and CD33-negative (CD33−) myeloid hematopoiesis are determined from the peripheral blood at this time. If the subject exhibits successful CD33-HSC engraftment and CD33-hematopoiesis at day 60, the subject is subsequently administered gemtuzumab ozogamicin/Mylotarg®. The CD33-ANC is monitored in the subject prior to administration of gemtuzumab ozogamicin/Mylotarg®, and the subject should preferably have ≥1000/dL CD33-ANC prior to receiving gemtuzumab ozogamicin/Mylotarg®.

Administration of gemtuzumab ozogamicin/Mylotarg® is preferably initiated within 30 days of the bone marrow biopsy at day 60, i.e., is preferably initiated by day 90. However, initiation of gemtuzumab ozogamicin/Mylotarg® may be delayed up to day 120 if a subject's clinical status, e.g., in view of comorbidities, including, for example, HCT-related comorbidities, necessitate such a delay, or in order to allow attainment of ≥1000/dL CD33-ANC in a subject. If gemtuzumab ozogamicin/Mylotarg® is initiated more than 30 days after the Day 60 bone marrow biopsy, a repeat bone marrow biopsy is completed prior to starting gemtuzumab ozogamicin/Mylotarg®.

Gemtuzumab ozogamicin/Mylotarg® is administered to the subject at a dose within the range of 0.1 mg/m² to 2 mg/m², e.g., at a dose of 0.1 mg/m², 0.25 mg/m², 0.5 mg/m², 1 mg/m², or 2 mg/m². A dose of 2 mg/m² of gemtuzumab ozogamicin/Mylotarg® is preferred for most subjects. However, some subjects may be administered a lower dose, e.g., in the event of treatment-related adverse effects, e.g., dose-limited toxicities (DLT), or in view of the health status, comorbidities, or the medical history of the individual subject.

Gemtuzumab ozogamicin/Mylotarg® is administered to the subject in a regimen of 4-week (28d) treatment cycles, wherein the subject receives the entire dose of a respective treatment cycle, e.g., at 2 mg/m² of gemtuzumab ozogamicin/Mylotarg®, on day 1 of the respective 4-week (28d) treatment cycle.

For most subjects, four consecutive treatment cycles of gemtuzumab ozogamicin/Mylotarg®, and thus four doses of gemtuzumab ozogamicin/Mylotarg®, spread 4 weeks apart from each other, are preferred. However, in some subjects, additional gemtuzumab ozogamicin/Mylotarg® treatment cycles may be indicated, e.g., up to four additional “continuation” treatment cycles, e.g., at the same dose of the initial four treatment cycles, or at a lower dose, if clinically advisable, e.g., based on the subject's clinical status.

At completion of the last gemtuzumab ozogamicin/Mylotarg® treatment cycle, the subject is monitored for disease status and hematopoietic chimerism and is monitored for these parameters every six months for five years after completion of the final treatment cycle.

### Example 7: Treatment of a Subject Having AML with CD33KO eHSPC Generated Using gRNA E, and the CD33-Targeted ADC Gemtuzumab Ozogamicin/Mylotarg®

A subject having CD33-positive AML is treated with an allogeneic HCT comprising CD33KO eHSPCs and with the ADC gemtuzumab ozogamicin/Mylotarg®.

For the HCT, a population of cells comprising CD34+ hematopoietic stem cells is obtained from a healthy donor who is HLA matched at 8/8 loci (HLA-A, —B, —C, DRB1) to the subject.

After G-CSF/plerixafor mobilization, up to two apheresis procedures are performed in order to obtain a minimum of 10×10⁶ viable cells/kg (where kg refers to recipient subject weight) from the donor for processing and subsequent administration to the recipient subject. From this apheresis product, at least 3.0×10⁶ viable cells/kg (recipient weight) undergo minimal manipulation and are cryopreserved to serve as a back-up stem cell source, e.g., for use as a rescue dose. The remainder of the apheresis product is used for processing and preparation of the CD33KO eHSPC population for HCT.

The CD33KO eHSPC population for HCT is prepared by enriching the apheresis product for CD34+ cells, followed by electroporation and editing with a CD33 gRNA/Cas9 complex, as described in Example 1, using a Cas9 sgRNA comprising the nucleotide sequence 5′-CCUCACUAGACUUGACCCACGUUUUAGAGCUAGAAAUAGCAAGUUAAAAU AAGGCUAGUCCGUUAUCAACUUGAAAAAGUGGCACCGAGUCGGUGCUUUU-3′ (SEQ ID NO: 23, targeting domain sequence in bold, chemical modifications as provided in Example 1, see SEQ ID NO: 25).

The edited cells are subsequently placed in culture for <48 hours. Upon harvest, after the culture duration is finished, cells are washed, resuspended in the final formulation, and cryopreserved. Cell viability and editing efficiency are confirmed using a representative sample as described in Example 1, and CD33KO eHSPC populations meeting the criteria set forth in Example 1 (at least 70% viability and at least 45% CD33 editing efficiency) are used for HCT. A population for administration to a subject comprises a CD33KO eHSPC population satisfying these viability and editing efficiency criteria of at least 3×10⁶ cells/kg body weight of the recipient subject, and preferably comprises at least 4×10⁶ cells/kg, 5×10⁶ cells/kg, 6×10⁶ cells/kg, or 7×10⁶ cells/kg of the recipient subject.

After completion of the conditioning regimen, the subject receives an HCT comprising the thawed CD33KO eHSPCs via an intravenous (IV) infusion. The day of the HCT is day 0 of the treatment regimen.

The subject is assessed for CD33KO eHSPC engraftment at day 28 by measuring the absolute peripheral neutrophil count (ANC) for CD33KO (CD33−) neutrophils in the subject. The subject is deemed to exhibit neutrophil recovery (also referred to as successful CD33KO neutrophil engraftment) if the subject exhibits an absolute peripheral CD33KO neutrophil count of ≥1000/dL CD33-ANC at 28 days after CD33KO eHSPC HCT.

If the subject exhibits neutrophil recovery at day 28, a bone marrow biopsy is obtained from the subject on day 60 in order to assess disease status and hematopoietic recovery. In addition, percent donor chimerism and CD33-negative (CD33−) myeloid hematopoiesis are determined from the peripheral blood at this time. If the subject exhibits successful CD33-HSC engraftment and CD33-hematopoiesis at day 60, the subject is subsequently administered gemtuzumab ozogamicin/Mylotarg®. The CD33-ANC is monitored in the subject prior to administration of gemtuzumab ozogamicin/Mylotarg®, and the subject should preferably have ≥1000/dL CD33-ANC prior to receiving gemtuzumab ozogamicin/Mylotarg®.

Administration of gemtuzumab ozogamicin/Mylotarg® is preferably initiated within 30 days of the bone marrow biopsy at day 60, i.e., is preferably initiated by day 90. However, initiation of gemtuzumab ozogamicin/Mylotarg® may be delayed up to day 120 if a subject's clinical status, e.g., in view of comorbidities, including, for example, HCT-related comorbidities, necessitate such a delay, or in order to allow attainment of ≥1000/dL CD33-ANC in a subject. If gemtuzumab ozogamicin/Mylotarg® is initiated more than 30 days after the Day 60 bone marrow biopsy, a repeat bone marrow biopsy is completed prior to starting gemtuzumab ozogamicin/Mylotarg®.

Gemtuzumab ozogamicin/Mylotarg® is administered to the subject at a dose within the range of 0.1 mg/m² to 2 mg/m², e.g., at a dose of 0.1 mg/m², 0.25 mg/m², 0.5 mg/m², 1 mg/m², or 2 mg/m². A dose of 2 mg/m² of gemtuzumab ozogamicin/Mylotarg® is preferred for most subjects. However, some subjects may be administered a lower dose, e.g., in the event of treatment-related adverse effects, e.g., dose-limited toxicities (DLT), or in view of the health status, comorbidities, or the medical history of the individual subject.

Gemtuzumab ozogamicin/Mylotarg® is administered to the subject in a regimen of 4-week (28d) treatment cycles, wherein the subject receives the entire dose of a respective treatment cycle, e.g., at 2 mg/m² of gemtuzumab ozogamicin/Mylotarg®, on day 1 of the respective 4-week (28d) treatment cycle.

For most subjects, four consecutive treatment cycles of gemtuzumab ozogamicin/Mylotarg®, and thus four doses of gemtuzumab ozogamicin/Mylotarg®, spread 4 weeks apart from each other, are preferred. However, in some subjects, additional gemtuzumab ozogamicin/Mylotarg® treatment cycles may be indicated, e.g., up to four additional “continuation” treatment cycles, e.g., at the same dose of the initial four treatment cycles, or at a lower dose, if clinically advisable, e.g., based on the subject's clinical status.

At completion of the last gemtuzumab ozogamicin/Mylotarg® treatment cycle, the subject is monitored for disease status and hematopoietic chimerism and is monitored for these parameters every six months for five years after completion of the final treatment cycle.

### Example 8: Combination Treatment

Subjects having acute myeloid leukemia (AML) are matched with a healthy stem cell donor based on 8/8 loci (HLA-A, -B, -C, DRB1). Up to two apheresis procedures are performed on the donor subject in order to obtain a minimum of 10×10⁶ viable cells/kg for the recipient subject.

At least about 10×10⁶ viable cells/kg for the recipient of CD34+ healthy donor cells are used to manufacture the CD33KO eHSPC HCT product and a minimum of 3.0×10⁶ viable cells/kg to produce the back-up graft (i.e., rescue dose). The CD33KO eHSPC HCT manufacturing process consists of CD34+ cell enrichment followed by electroporation and editing with a CD33 gRNA/Cas9 complex, for example using any of the CD33 gRNAs described herein, see Table 1. The edited cells are subsequently cultured for <48 hours. Upon harvest, after the culture duration is finished, cells are washed, resuspended in the final formulation, and cryopreserved.

The recipient subject may undergo a myeloablative conditioning regimen (preconditioning) prior to administration of the CD33 edited hematopoietic cells. After completion of the conditioning regimen, the subject is administered the CD33KO eHSPC HCT on day 0 via an intravenous (IV) infusion. The subject is monitored for neutrophil recovery, which is defined as recovery of peripheral neutrophil count at 28 days following infusion. At day 60, if the subject has successful neutrophil engraftment, the subject receives a bone marrow biopsy to assess disease status and hematopoietic recovery. The percent donor chimerism and CD33-negative (CD33−) myeloid hematopoiesis are determined from the peripheral blood. Subjects must have a neutrophil count above a threshold (e.g., ≥1000/dL CD33-ANC) prior to receiving gemtuzumab ozogamicin/Mylotarg®. The subjects then receive gemtuzumab ozogamicin/Mylotarg® at a dose between about 0.1 mg/m² to 2.0 mg/m².

The subjects are continued to be evaluated and may receive one or more additional doses of gemtuzumab ozogamicin/Mylotarg® at the same dosage as the previous dose or at a different (increased or decreased) dosage.

### Example 9: Combination Treatment Using Multiplexed Editing

Subjects having acute myeloid leukemia (AML) are matched with a healthy stem cell donor based on 8/8 loci (HLA-A, -B, -C, DRB1). Up to two apheresis procedures are performed on the donor subject in order to obtain a minimum of 10×10⁶ viable cells/kg for the recipient subject.

At least about 10×10⁶ viable cells/kg for the recipient of CD34+ healthy donor cells are used to manufacture the double edited hematopoietic cell product and a minimum of 3.0×10⁶ viable cells/kg to produce the back-up graft (i.e., rescue dose). The genetic editing manufacturing process consists of CD34+ cell enrichment followed by electroporation and editing with a CD33 gRNA/Cas9 complex, for example using any of the CD33 gRNAs described herein, see Table 1, as well as a gRNA/Cas9 complex targeting a second lineage-specific cell-surface antigen, such as any of the those described herein. The double edited cells are subsequently cultured for <48 hours. Cell surface levels of CD33 and the second lineage-specific cell-surface antigen may be assessed in the double edited cells, for example by flow cytometry.

Upon harvest, after the culture duration is finished, cells are washed, resuspended in the final formulation, and cryopreserved.

The recipient subject may undergo a myeloablative conditioning regimen (preconditioning) prior to administration of the double edited hematopoietic cells. After completion of the conditioning regimen, the subject is administered the double edited hematopoietic cells on day 0 via an intravenous (IV) infusion. The subject is monitored for neutrophil recovery, which is defined as recovery of peripheral neutrophil count at 28 days following infusion. At day 60, if the subject has successful neutrophil engraftment, the subject receives a bone marrow biopsy to assess disease status and hematopoietic recovery. The percent donor chimerism and CD33-negative (CD33−) myeloid hematopoiesis are determined from the peripheral blood. Subjects must have a neutrophil count above a threshold (e.g., ≥1000/dL CD33− ANC) prior to receiving gemtuzumab ozogamicin/Mylotarg®. The subjects then receive gemtuzumab ozogamicin/Mylotarg® at a dose between about 0.1 mg/m² to 2.0 mg/m².

The subjects are continued to be evaluated and may receive one or more additional doses of gemtuzumab ozogamicin/Mylotarg® at the same dosage as the previous dose or at a different (increased or decreased) dosage.

### Example 10: Xenotransplantation Model for Use of Human CD33KO eHSPC Generated Using gRNA E and the CD33-Targeted ADC Gemtuzumab Ozogamicin/Mylotarg®

The objective of this study was to investigate whether human hematopoietic stem and progenitor cells (HSPCs) that have been genetic engineered to have reduced or eliminated expression of CD33, or their derivative cells (descendants thereof), are protected against the cytotoxicity of gemtuzumab ozogamicin/Mylotarg® using a mouse model.

Mobilized PBMCs were obtained from two human donors (Donor 1 and Donor 2) and screened to confirm that the cells are not homozygous for a single nucleotide polymorphism (SNP) at rs12459419, which causes an alternatively spliced transcript variant lacking exon 2, resulting in decreased expression of full-length CD33 isoform, and would therefore not be recognized or targeted by gemtuzumab ozogamicin/Mylotarg®.

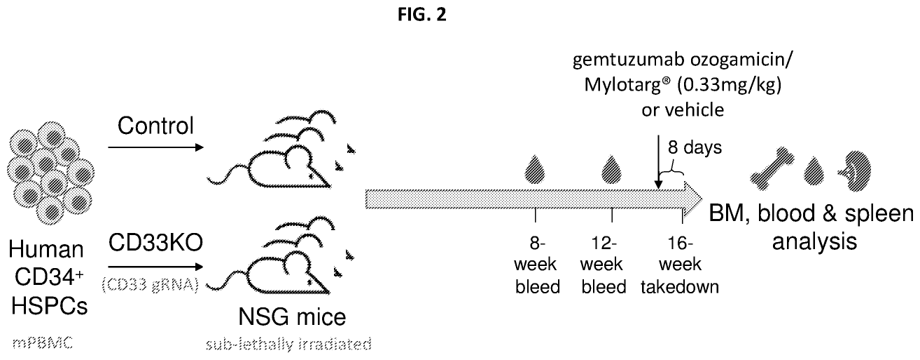

Briefly, as shown in FIG. 2, CD34+ HSPCs from the donor were electroporated with CD33 gRNA E Cas9 ribonucleoprotein complex, as described in Example 1. Cells from the same donor were electroporated without RNP were used as negative controls (“Mock EP”). CD33 editing efficiency is shown in Table 4. Cell number and viability were also quantified prior to and after electroporation (Table 5).

TABLE 4
CD33 editing efficiency of human CD34+ HSPCs

| Donor | gRNA | Editing Frequency |
|---|---|---|
| Donor 1 | CD33 gRNA-E | 86% |
| Donor 2 | CD33 gRNA-E | 76% |

TABLE 5
Cell numbers and viability

| | Pre-EP | | 48 Hours Post-EP | | | |
|---|---|---|---|---|---|---|
| | | Viable | Mock EP | | CD33KO | |
| | | Cell # per | | Viable | | Viable |
| Donor | Viability | Condition | Viability | Cell # | Viability | Cell # |
| Donor 1 | 98% | 7.2 × 10⁷ | 93% | 9.4 × 10⁷ | 94% | 9.4 × 10⁷ |
| Donor 2 | 97% | 6.0 × 10⁷ | 88% | 5.5 × 10⁷ | 87% | 4.8 × 10⁷ |

CD33-edited HSPCs (CD33KO) and Mock EP control CD34+ HSPCs were injected via tail vein into sub-lethally irradiated NOD/scid/IL2R7^{null} ((NOD.Cg-Prkdc^{scid}Il2rg^{tm1Wjl}, NSG™) mice. At 8 weeks post-transplantation, retro-orbital blood was collected from each mouse for flow cytometry analysis to assess engraftment of CD34+ HSPCs. Retro-orbital bleeding was also performed at 12 weeks post-transplantation to collect plasma and cell pellets, which were stored for further analysis. At 15 weeks post-transplantation, after hematopoietic reconstitution by transplanted human HSPCs, the mice were dosed intravenously with either gemtuzumab ozogamicin/Mylotarg® (0.33 mg/kg) or DPBS (Dulbecco's phosphate-buffered saline) as the vehicle control (“vehicle”). Eight days post-treatment with gemtuzumab ozogamicin/Mylotarg® or vehicle (at 16 weeks after transplantation), mice were euthanized and bone marrow, blood, and spleens were harvested for flow cytometry analysis. The experimental groupings for cells obtained from Donor 1 and Donor 2 are shown in Tables 6 and 7, respectively.

TABLE 6
Experimental groupings for cells obtained from Donor 1

| Group | Number of Mice | mPB CD34⁺ HSPCs | Treatment |
|---|---|---|---|
| 1 | 16 | CD33 gRNA-Cas9 | Vehicle |
| 2 | 16 | RNP | Mylotarg (0.33 |
| | | | mg/kg) |
| 3 | 16^{#} | Mock EP control | Vehicle |
| 4 | 16 | | Mylotarg (0.33 |
| | | | mg/kg) |

TABLE 7
Experimental groupings for cells obtained from Donor 2

| Group | Number of Mice | mPB CD34⁺ HSPCs | Treatment |
|---|---|---|---|
| 1 | 16 | Mock EP control | Vehicle |
| 2 | 16 | | Mylotarg (0.33 |
| | | | mg/kg) |
| 3 | 16 | CD33 gRNA-Cas9 | Vehicle |
| 4 | 16 | RNP | Mylotarg (0.33 |
| | | | mg/kg) |

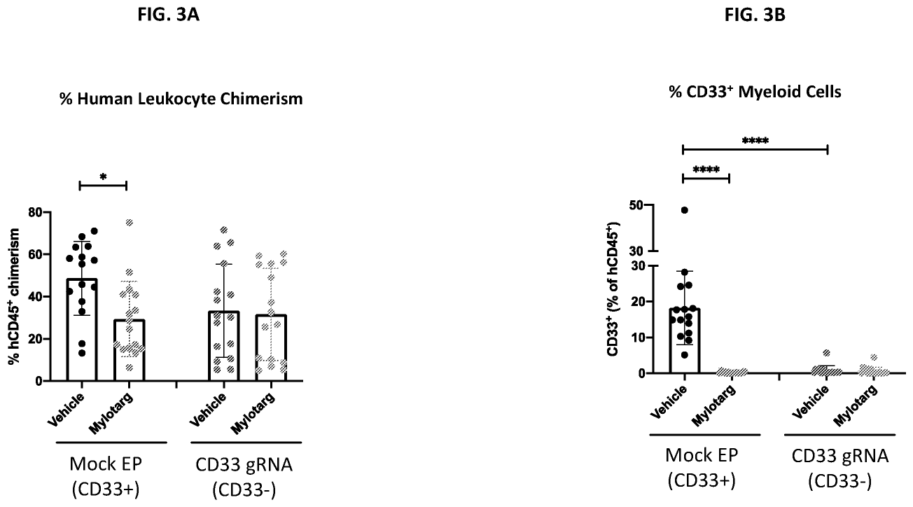

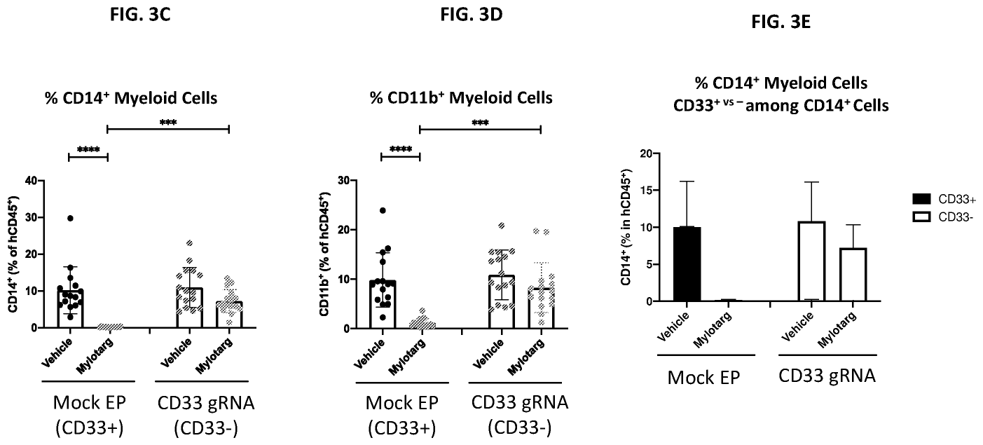

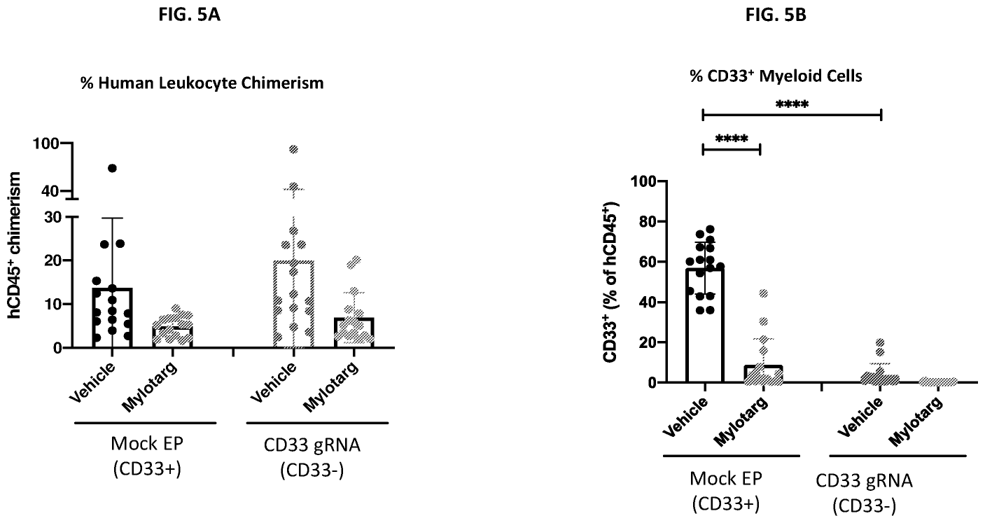

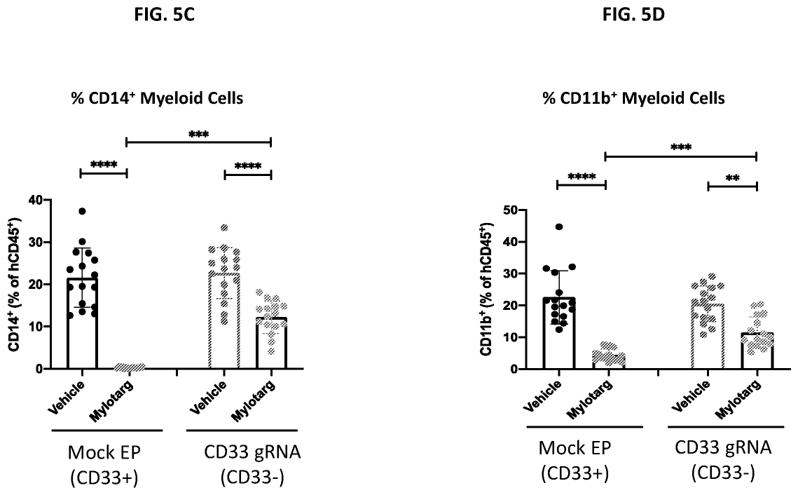

As shown in FIG. 3A, for mice engrafted with Mock EP HSPCs from Donor 1, human leukocyte reconstitution (hCD45+ cells) was reduced by gemtuzumab ozogamicin/Mylotarg® treatment, for mice engrafted with CD33-edited HSPCs from Donor 1 were not impacted by gemtuzumab ozogamicin/Mylotarg®. Additionally, CD33 gene-editing did not result in a significant change in human cell leukocyte chimerism as evidenced by human CD45 staining. For mice engrafted with HSPCs from Donor 2, no statistically significant difference was observed in human cell chimerism between groups that received gemtuzumab ozogamicin/Mylotarg® treatment versus vehicle control, or comparing mice engrafted with CD33-edited HSPCs versus Mock EP HSPCs (FIG. 5A).

The percentages of CD33+ cells among total human leukocytes (hCD45+ cells) in the bone marrow of engrafted animals were analyzed by flow cytometry. For HSPCs from each donor, mice that were engrafted with the Mock EP HSPCs followed by treatment with gemtuzumab ozogamicin/Mylotarg® had a significant reduction of CD33+ cells, as compared to treatment with vehicle alone (FIGS. 3B, 5B). Moreover, mice engrafted with CD33-edited HSPCs showed a nearly complete loss of CD33+ cells, as compared to mice engrafted with Mock EP HSPCs, regardless of treatment with gemtuzumab ozogamicin/Mylotarg® or vehicle control (FIGS. 3B, 5B). Without wishing to be bound by any particular theory, these results are thought to be due to highly efficient editing of donor human HSPCs and demonstrate robust ablation of CD33+ cells by gemtuzumab ozogamicin/Mylotarg®. This suggests there is long-term persistence of CD33-edited HSPCs cells in this xenotransplant model.

Cells expressing different human myeloid markers were also evaluated from the human CD45+ (mouse CD45−) population. The fractions of monocytes (CD14+ myeloid cells) in total human leukocytes (hCD45+) in the bone marrow of engrafted mice were analyzed. For both donors, gemtuzumab ozogamicin/Mylotarg® treatment led to a near complete elimination of CD14+ myeloid cells in the mice engrafted with Mock EP HSPCs group (FIGS. 3C, 5C). In contrast, gemtuzumab ozogamicin/Mylotarg® treatment had less of an impact on the percentage of CD14+ myeloid cells in mice that were transplanted with CD33-edited HSPCs (FIGS. 3C, 5C). These results indicate that the CD33-edited myeloid cells were protected from the cytotoxicity of gemtuzumab ozogamicin/Mylotarg® (FIG. 3E). No significant difference was observed in the percentage of CD14+ myeloid cells between mice engrafted with Mock EP HSPCs and those engrafted CD33-edited HSPCs, supporting that loss of CD33 does not compromise long-term differentiation of CD14+ myeloid cells from HSPCs.

CD11b+ myeloid cells within the hCD45+ population of cells were also analyzed. For mice engrafted with Mock EP HSPCs from either donor, gemtuzumab ozogamicin/Mylotarg® treatment eradicated the majority of CD11b+ myeloid cells (FIGS. 3D, 5D). In contrast, gemtuzumab ozogamicin/Mylotarg® treatment had less of an impact on the percentage of CD11b+ myeloid cells in mice that were transplanted with CD33-edited HSPCs (FIGS. 3D, 5D). These results support that reduction in CD33 protects the CD11b+ myeloid cells against gemtuzumab ozogamicin/Mylotarg®. No significant difference was observed in the percentage of CD11b+ myeloid cells between mice engrafted with Mock EP HSPCs and those engrafted CD33-edited HSPCs, supporting that loss of CD33 does not compromise long-term differentiation of CD11b+ myeloid cells from HSPCs.

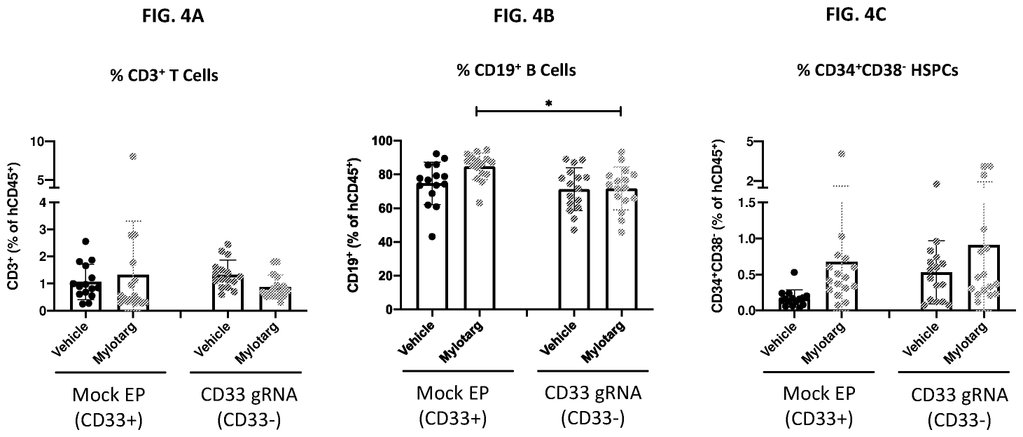

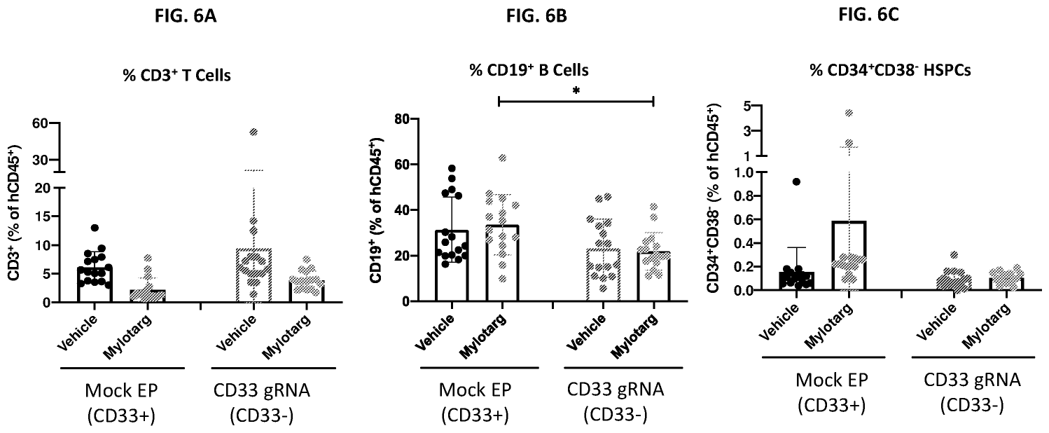

In addition to myeloid cells, the percentages of CD3+ T cells among total human CD45+ cells in the bone marrow of engrafted mice were analyzed. No statistically significant difference was observed in the percentage of CD3+ T cells in mice that were transplanted with Mock EP HSPCs as compared to mice that were transplanted with CD33-edited HSPCs (FIGS. 4A, 6A). As expected, CD3+ T cells were not affected by gemtuzumab ozogamicin/Mylotarg® treatment.

The percentages of CD19+ B cells among total human CD45+ cells in the bone marrow of engrafted animals were analyzed. No statistically significant difference was observed in the percentage of CD19+ B cells in mice that were transplanted with Mock EP HSPCs as compared to mice that were transplanted with CD33-edited HSPCs (FIGS. 4B, 6B). As expected, CD19+ B cells were not affected by gemtuzumab ozogamicin/Mylotarg® treatment.

Comparing vehicle-treated groups, mice transplanted with CD33-edited HSPCs and Mock EP HSPCs had equivalent levels of B cells, suggesting that B cell differentiation from HSPCs is not disturbed by CD33KO. Comparing the mice that received gemtuzumab ozogamicin/Mylotarg® treatment, mice transplanted with Mock EP HSPCs had a higher fraction of CD19+ B cells among total human leukocytes than those transplanted with CD33-edited HSPCs (FIGS. 4B, 6B). This likely reflects that since myeloid and lymphoid fractions make up most of the total human leukocyte compartment in the bone marrow of this mouse model, a decrease in the myeloid proportion (due to gemtuzumab ozogamicin/Mylotarg® treatment) will result in an apparent proportional increase in the lymphoid fraction, in the mice that received Mock EP HSPCs.

In addition, the percentages of CD34+CD38− human primitive HSPCs were evaluated in the bone marrow of engrafted mice. No statistically significant difference was observed in the percentage of CD34+CD38− cells in mice that received Mock EP HSPCs versus CD33-edited HSPCs nor between mice that received gemtuzumab ozogamicin/Mylotarg® treatment versus vehicle control (FIGS. 4C, 6C). These results suggest that loss of CD33 does not impact CD34+CD38− primitive HSPCs, and that the primitive HSPCs are not targeted by gemtuzumab ozogamicin/Mylotarg® cytotoxicity.

In sum, these analyses indicate that CD33-deficient cells are protected from gemtuzumab ozogamicin/Mylotarg cytotoxicity. Upon engraftment, CD33-edited HSPCs reconstituted a multilineage hematopoietic system with equivalent levels of human leukocyte chimerism, lymphoid and myeloid lineages, and primitive HSPCs, as control HSPCs. Additionally, a significant loss of CD33+ cells was observed in mice engrafted with CD33-edited HSPCs, demonstrating highly efficient CD33 disruption after 16 weeks and long-term persistence of CD33-deficient cells. In mice engrafted with control (unedited) HSPCs, gemtuzumab ozogamicin/Mylotarg® treatment effectively eliminated CD14+ and CD11b+ myeloid cells. In contrast, a significantly higher level of myeloid cells was retained post gemtuzumab ozogamicin/Mylotarg® treatment in animals transplanted with CD33-edited HSPCs. Taken together, these findings demonstrate that depletion of CD33 confers substantial protection to myeloid cells against gemtuzumab ozogamicin/Mylotarg® cytotoxicity in vivo.

### Example 11: Clinical Scale Manufacturing of Human CD33KO eHSPC

Two clinical-scale batches of allogeneic CRISPR/Cas9 genome edited hematopoietic stem/progenitor cells (HSPCs) lacking the CD33 protein were manufactured for the treatment of human leukocyte antigen (HLA)-matched patients with high-risk CD33+ acute myeloid leukemia (AML). The resulting HSPC populations are suitable for infusion into HLA-matched human patients with AML undergoing hematopoietic cell transplant, e.g., patients who are known to be at high-risk for leukemia relapse and mortality post-transplantation.

The final HSPC populations were formulated at a volume of 45 mL in cryopreservation media ready for cryopreservation, storage in the vapor phase of liquid nitrogen, subsequent thawing and administration via intravenous (IV) infusion to a recipient patient.

Each batch was manufactured from leukapheresis starting material obtained from a single donor to generate a one-donor-to-one-recipient HLA matched product, allowing for the manufacture of the product for a specifically matched patient.

For each batch, a healthy donor was subjected to a leukapheresis procedure. Leukapheresis starting material was collected and stored at 2-8° C. before initiation of cell manipulation. Cell number and viability of the leukapheresis starting material was tested by flow cytometry. Cell viability was confirmed to be ≥80%. A sample was removed for cell analysis and other assessments.

A leukapheresis rescue dose was removed from the leukapheresis material to obtain a volume comprising 3×10⁶ CD34+ cells/kg of patient weight. The rescue dose material was cryopreserved and stored in the vapor phase of liquid nitrogen at ≤−140° C.

After rescue dose removal, the leukapheresis starting material was processed to remove red blood cells, platelets, and plasma. The processed material was then enriched for CD34-positive cells and then transferred into 250 mL conical tubes.

A Cas9/gRNA ribonucleoprotein (RNP) complex was prepared prior to electroporation by mixing Cas9 protein and gRNA E under sterile conditions. Cells in the 250 mL conical tube were spun down at 200×g, resuspended in electroporation buffer, mixed with the prepared RNP complex, and electroporated in a single-use sterile electroporation cassette.

Post-electroporation, the cells were removed from the cassette, transferred to culture media, and incubated in suspension culture at 37° C. and 5% CO₂. Cell cultures were monitored for cell count and viability. Once cells recovered from electroporation (determined by cell viability being ≥80%), the cells were washed to reduce cellular debris and other residuals, and resuspended in serum-free, animal component-free, and defined cryopreservation medium containing 10% DMSO. The cells were formulated at a volume of 45 mL and cryopreserved in a controlled rate freezer (CRF). Samples were taken to determine cell counts, viability, percentage of cells expressing particular markers (e.g., CD34, CD3, CD19, CD19, CD56), editing efficiency, and residual Cas9 as shown in Table 8 below.

TABLE 8
Analysis of exemplary cell preparations

| | BATCH 1 | | BATCH 2 | |
|---|---|---|---|---|
| Volume | 45 | ml | 45 | ml |
| Viable cell concentration | 6.5 × 10⁶ | cells/mL | 3.6 × 10⁶ | cells/mL |

| | | |
|---|---|---|
| Viability | 78% | 70% |
| CD34⁺ | 95% | 96% |
| CD3⁺ | 0% | 1% |
| CD19⁺ | 0.3% | 0.9% |
| CD14⁺ | 0.2% | 0.2% |
| CD56⁺ | 1.1% | 1.1% |
| CD33 edited | 67% | 64% |
| Residual Cas9 | BLLQ | BLLQ |

BLLQ = below lower limit of quantification.

### Enumerated Embodiments

1. A method, comprising administering to a subject:
an effective amount of a population of genetically engineered hematopoietic cells, or descendants thereof, comprising a modified gene encoding CD33 that is engineered to have reduced or eliminated expression of a CD33 antigen; and
an effective amount of a cytotoxic agent comprising an anti-CD33 antigen-binding domain.
   2. The method of embodiment 1, wherein the cytotoxic agent is an antibody-drug conjugate (ADC).
   3. The method of embodiment 2, wherein the ADC is gemtuzumab ozogamicin.
   4. The method of any one of the preceding embodiments, wherein the effective amount of the population of genetically engineered hematopoietic cells is about 10⁶ cells/kilogram body weight of the subject to about 10⁷ cells/kilogram body weight of the subject.
   5. The method of embodiment 4, wherein the effective amount of the population of genetically engineered hematopoietic cells is about 3.0×10⁶ cells/kilogram body weight of the subject.
   6. The method of any one of the preceding embodiments, wherein the effective amount of the cytotoxic agent is about 0.1 mg/m² body surface area of the subject to about 2.0 mg/m² body surface area of the subject.
   7. The method of embodiment 6, wherein the effective amount of the cytotoxic agent is about 0.1 mg/m², about 0.25 mg/m², about 0.5 mg/m², about 1.0 mg/m², or about 2.0 mg/m² body surface area of the subject.
   8. The method of embodiment 7, wherein the effective amount of the cytotoxic agent is about 2.0 mg/m² body surface area of the subject.
   9. The method of any one of the preceding embodiments, wherein the population of genetically engineered hematopoietic cells and the cytotoxic agent are administered in temporal proximity.
   10. The method of embodiment 9, wherein administering in temporal proximity comprises administering the population of genetically engineered hematopoietic cells and the cytotoxic agent within a single treatment regimen.
   11. The method of embodiment 9, wherein administering in temporal proximity comprises administering the population of genetically engineered hematopoietic cells and the cytotoxic agent simultaneously.
   12. The method of embodiment 9, wherein administering in temporal proximity comprises administering the population of genetically engineered hematopoietic cells and the cytotoxic agent concurrently.
   13. The method of embodiment 9, wherein administering in temporal proximity comprises administering the population of genetically engineered hematopoietic cells and the cytotoxic agent sequentially.
   14. The method of embodiment 9, wherein administering in temporal proximity comprises administering the population of genetically engineered hematopoietic cells within 120 days of administering the cytotoxic agent.
   15. The method of embodiment 14, wherein administering in temporal proximity comprises administering the population of genetically engineered hematopoietic cells within 90 days of administering the cytotoxic agent.
   16. The method of embodiment 15, wherein administering in temporal proximity comprises administering the population of genetically engineered hematopoietic cells within 60 days of administering the cytotoxic agent.
   17. The method of any one of embodiments 1-9 or 12-16, wherein the population of genetically engineered hematopoietic cells are administered prior to the cytotoxic agent.
   18. The method of any one of the preceding embodiments, wherein the population of genetically engineered hematopoietic cells are administered in a single treatment regimen.
   19. The method of any one of the preceding embodiments, wherein the population of genetically engineered hematopoietic cells and/or the cytotoxic agent are administered intravenously.
   20. The method of any one of the preceding embodiments, wherein the cytotoxic agent is administered in multiple doses of the effective amount every four weeks.
   21. The method of any one of the preceding embodiments, wherein the cytotoxic agent is administered in multiple doses of about 2.0 mg/m² every four weeks.
   22. The method of any one of the preceding embodiments, wherein the population of genetically engineered hematopoietic cells are thawed from a cryopreserved form prior to administration.
   23. The method of any one of the preceding embodiments, wherein the cytotoxic agent is reconstituted from a lyophilized form prior to administration.
   24. The method of any one of the preceding embodiments, wherein the subject has been preconditioned prior to administering the cytotoxic agent and/or the hematopoietic cells.
   25. The method of any one of the preceding embodiments, further comprising preconditioning the subject prior to administering the cytotoxic agent and/or the hematopoietic cells.
   26. The method of embodiment 24 or embodiment 25, wherein the preconditioning comprises administering one or more chemotherapeutic agents to the subject.
   27. The method of any one of embodiments 24-26, wherein the preconditioning comprises total body irradiation of the subject.
   28. The method of any one of embodiments 24-27, wherein the chemotherapeutic agent is selected from the group consisting of busulfan, melphalan, fludarabine, cyclophosphamide, and thiotepa.
   29. The method of any one of embodiments 24-28, wherein the preconditioning comprises administering antibodies that bind human T cells, optionally wherein the antibodies comprise rabbit anti-thymocyte globulins (rATG).
   30. The method of any one of the preceding embodiments, wherein the subject has, or has been diagnosed with, a hematopoietic malignancy or a hematopoietic pre-malignant disease, and wherein the hematopoietic malignancy is characterized by the presence of CD33-positive malignant cells, or wherein the hematopoietic pre-malignant disease is characterized by the presence of CD33-positive pre-malignant cells.
   31. The method of any one of the preceding embodiments, wherein the subject has, or has been diagnosed with, CD33-positive acute myeloid leukemia.
   32. The method of any one of the preceding embodiments, wherein the subject has, or has been diagnosed with, CD33-positive myelodysplastic syndrome.
   33. The method of any one of embodiments 1-30, wherein the subject has, or has been diagnosed with, CD33-positive myelodysplastic syndrome and wherein the subject is at high risk of developing acute myeloid leukemia.
   34. The method of any one of the preceding embodiments, wherein the subject is naïve to chemotherapy and/or radiation therapy, optionally wherein the subject is naïve to any treatment aimed to address a hematopoietic malignancy or hematopoietic pre-malignant disease.
   35. The method of any one of the preceding embodiments, wherein the subject has previously received chemotherapy.
   36. The method of any one of the preceding embodiments, wherein the subject has previously received induction therapy.
   37. The method of any one of the preceding embodiments, wherein the subject has previously entered a complete hematological remission, optionally wherein the complete hematological remission is characterized by an incomplete recovery of peripheral counts.
   38. The method of any one of the preceding embodiments, wherein the subject has one or more risk factors associated with early leukemia relapse.
   39. The method of embodiment 38, wherein the one or more risk factors associated with early leukemia relapse are selected from the group consisting of: bone marrow in morphological complete remission with presence of intermediate or high-risk disease-related genetics; presence of minimal residual disease (MRD) post cyto-reductive therapy; bone marrow with persistent leukemia blasts post cyto-reductive therapy; and bone marrow blast count of about 10% or less with no circulating blasts.
   40. The method of any one of the preceding embodiments, wherein the subject does not have a homozygous dominant genotype for CD33 single nucleotide polymorphism (SNP) rs12459419.
   41. The method of any one of the preceding embodiments, wherein the subject does not have acute promyelocytic leukemia or chronic myeloid leukemia.
   42. The method of any one of the preceding embodiments, wherein the subject does not have a genetic translocation associated with acute promyelocytic leukemia or chronic myeloid leukemia, optionally wherein the genetic translocation is t(15; 17)(q22; q21) or t(9; 22)(q34; q11).
   43. The method of any one of the preceding embodiments, wherein the subject has not previously received a stem cell transplantation.
   44. The method of any one of the preceding embodiments, wherein the subject has not previously received the cytotoxic agent.
   45. The method of any one of the preceding embodiments, further comprising determining a percent donor chimerism and/or a level of CD33-negative myeloid hematopoiesis in a peripheral blood sample from the subject.
   46. The method of any one of the preceding embodiments, wherein the subject has a CD33-negative absolute neutrophil count (ANC) of at least 1000/dL prior to receiving the cytotoxic agent.
   47. The method of any one of the preceding embodiments, wherein the hematopoietic cells are hematopoietic stem cells.
   48. The method of embodiment 47, wherein the hematopoietic stem cells are from bone marrow cells, cord blood cells, or peripheral blood mononuclear cells (PBMCs).
   49. The method of embodiment 47 or embodiment 48, wherein the hematopoietic stem cells are CD34+/CD33−.
   50. The method of any one of the preceding embodiments, wherein the hematopoietic cells are autologous.
   51. The method of embodiment 50, wherein the method further comprises obtaining the autologous hematopoietic stem cells from the subject, optionally wherein the method further comprises genetically engineering the autologous stem cells to have reduced or eliminated expression of the CD33 antigen, and returning the genetically engineered hematopoietic stem cells to the subject.
   52. The method of any one of the preceding embodiments, wherein the hematopoietic cells are allogeneic.
   53. The method of embodiment 52, wherein the hematopoietic cells are allogeneic hematopoietic stem cells obtained from a donor having an HLA haplotype that matches with the HLA haplotype of the subject.
   54. The method of any one of the preceding embodiments, further comprising obtaining hematopoietic cells from a donor having an HLA haplotype that matches with the HLA haplotype of the subject.
   55. The method of any one of the preceding embodiments, further comprising preparing the hematopoietic cells by modifying an endogenous gene of the hematopoietic cells encoding the CD33 antigen.
   56. The method of embodiment 55, wherein the whole or a portion of the endogenous gene encoding the CD33 cell-surface antigen is deleted.
   57. The method of embodiment 55 or embodiment 56, wherein the whole or the portion of the endogenous gene is deleted using genome editing.
   58. The method of embodiment 57, wherein the genome editing involves a zinc finger nuclease (ZFN), a transcription activator-like effector-based nuclease (TALEN), or a CRISPR-Cas system.
   59. A method, comprising administering to a subject: an effective amount of a cytotoxic agent comprising an anti-CD33 antigen-binding domain, wherein the subject is receiving or has received an effective amount of a population of genetically modified hematopoietic cells, or descendants thereof, comprising a modified gene encoding CD33 that is engineered to have reduced or eliminated expression of a CD33 agent.
   60. The method of embodiment 59, wherein the cytotoxic agent is an antibody-drug conjugate (ADC).
   61. The method of embodiment 60, wherein the ADC is gemtuzumab ozogamicin.
   62. The method of any one of embodiments 59-61, wherein the effective amount of the population of genetically engineered hematopoietic cells is about 10⁶ cells/kilogram body weight of the subject to about 10⁷ cells/kilogram body weight of the subject.
   63. The method of embodiment 62, wherein the effective amount of the population of genetically engineered hematopoietic cells is about 3.0×10⁶ cells/kilogram body weight of the subject.
   64. The method of any one of embodiments 59-63, wherein the effective amount of the cytotoxic agent is about 0.1 mg/m² body surface area of the subject to about 2.0 mg/m² body surface area of the subject.
   65. The method of embodiment 64, wherein the effective amount of the cytotoxic agent is about 0.1 mg/m², about 0.25 mg/m², about 0.5 mg/m², about 1.0 mg/m², or about 2.0 mg/m² body surface area of the subject.
   66. The method of embodiment 65, wherein the effective amount of the cytotoxic agent is about 2.0 mg/m² body surface area of the subject.
   67. The method of any one of embodiments 59-66, wherein the effective amount of the cytotoxic agent is administered in temporal proximity with the effective amount of the population of genetically engineered hematopoietic cells.
   68. The method of embodiment 67, wherein administering in temporal proximity comprises administering the population of genetically engineered hematopoietic cells and the cytotoxic agent within a single treatment regimen.
   69. The method of embodiment 67, wherein administering in temporal proximity comprises administering the population of genetically engineered hematopoietic cells and the cytotoxic agent simultaneously.
   70. The method of embodiment 67, wherein administering in temporal proximity comprises administering the population of genetically engineered hematopoietic cells and the cytotoxic agent concurrently.
   71. The method of embodiment 67, wherein administering in temporal proximity comprises administering the population of genetically engineered hematopoietic cells and the cytotoxic agent sequentially.
   72. The method of embodiment 67, wherein administering in temporal proximity comprises administering the cytotoxic agent within 120 days of administering the population of genetically engineered hematopoietic cells.
   73. The method of embodiment 67, wherein administering in temporal proximity comprises administering the cytotoxic agent within 90 days of administering the population of genetically engineered hematopoietic cells.
   74. The method of embodiment 67, wherein administering in temporal proximity comprises administering the cytotoxic agent within 60 days of administering the hematopoietic cells. population of genetically engineered hematopoietic cells 75. The method of any one of embodiments 59-74, wherein the population of genetically engineered hematopoietic cells are administered prior to the cytotoxic agent.
   76. The method of any one of embodiments 59-75, wherein the population of genetically engineered hematopoietic cells are administered in a single treatment regimen.
   77. The method of one of embodiments 59-76, wherein the population of genetically engineered hematopoietic cells and/or the cytotoxic agent are administered intravenously.
   78. The method of any one of embodiments 59-77, wherein the cytotoxic agent is administered in multiple doses of the effective amount every four weeks.
   79. The method of any one of embodiments 59-78, wherein the cytotoxic agent is administered in multiple doses of about 2.0 mg/m² every four weeks.
   80. The method of any one of embodiments 59-79, wherein the population of genetically engineered hematopoietic cells are thawed from a cryopreserved form prior to administration.
   81. The method of any one of embodiments 59-80, wherein the cytotoxic agent is reconstituted from a lyophilized form prior to administration.
   82. The method of any one of embodiments 59-81, wherein the subject has been preconditioned prior to administering the cytotoxic agent and/or the hematopoietic cells.
   83. The method of any one of embodiments 59-82 further comprising preconditioning the subject prior to administering the cytotoxic agent and/or the population of genetically engineered hematopoietic cells.
   84. The method of embodiment 82 or embodiment 83, wherein the preconditioning comprises administering one or more chemotherapeutic agents to the subject.
   85. The method of any one of embodiments 82-84, wherein the preconditioning comprises total body irradiation of the subject.
   86. The method of any one of embodiments 82-85, wherein the chemotherapeutic agent is selected from the group consisting of busulfan, melphalan, fludarabine, cyclophosphamide, and thiotepa.
   87. The method of any one of embodiments 82-86, wherein the preconditioning comprises administering antibodies that bind human T cells, optionally wherein the antibodies comprise rabbit anti-thymocyte globulins (rATG).
   88. The method of any one of embodiments 59-87, wherein the subject has, or has been diagnosed with, a hematopoietic malignancy or a hematopoietic pre-malignant disease, and wherein the hematopoietic malignancy is characterized by the presence of CD33-positive malignant cells, or wherein the hematopoietic pre-malignant disease is characterized by the presence of CD33-positive pre-malignant cells.
   89. The method of any one of embodiments 59-88, wherein the subject has, or has been diagnosed with, CD33-positive acute myeloid leukemia.
   90. The method of any one of embodiments 59-88, wherein the subject has, or has been diagnosed with, CD33-positive myelodysplastic syndrome.
   91. The method of any one of embodiments 59-88, wherein the subject has, or has been diagnosed with, CD33-positive myelodysplastic syndrome and wherein the subject is at high risk of developing acute myeloid leukemia.
   92. The method of any one of embodiments 59-91, wherein the subject is naïve to chemotherapy and/or radiation therapy, optionally wherein the subject is naïve to any treatment aimed to address a hematopoietic malignancy or hematopoietic pre-malignant disease.
   93. The method of any one of embodiments 59-91, wherein the subject has previously received chemotherapy.
   94. The method of any one of embodiments 59-93, wherein the subject has previously received induction therapy.
   95. The method of any one of embodiments 59-94, wherein the subject has previously entered a complete hematological remission, optionally wherein the complete hematological remission is characterized by an incomplete recovery of peripheral counts.
   96. The method of any one of embodiments 59-95, wherein the subject has one or more risk factors associated with early leukemia relapse.
   97. The method of embodiment 96, wherein the one or more risk factors associated with early leukemia relapse are selected from the group consisting of: bone marrow in morphological complete remission with presence of intermediate or high-risk disease-related genetics; presence of minimal residual disease (MRD) post cyto-reductive therapy; bone marrow with persistent leukemia blasts post cyto-reductive therapy; and bone marrow blast count of about 10% or less with no circulating blasts.
   98. The method of any one of embodiments 59-97, wherein the subject does not have a homozygous dominant genotype for CD33 single nucleotide polymorphism (SNP) rs12459419.
   99. The method of any one of embodiments 59-98, wherein the subject does not have acute promyelocytic leukemia or chronic myeloid leukemia.
   100. The method of any one of embodiments 59-99, wherein the subject does not have a genetic translocation associated with acute promyelocytic leukemia or chronic myeloid leukemia, optionally wherein the genetic translocation is t(15; 17)(q22; q21) or t(9; 22)(q34; q11).
   101. The method of any one of embodiments 59-100, wherein the subject has not previously received a stem cell transplantation.
   102. The method of any one of embodiments 59-101, wherein the subject has not previously received the cytotoxic agent.
   103. The method of any one of embodiments 59-102, further comprising determining a percent donor chimerism and/or a level of CD33-negative myeloid hematopoiesis in a peripheral blood sample from the subject.
   104. The method of any one of embodiments 59-103, wherein the subject has a CD33-negative absolute neutrophil count (ANC) of at least 1000/dL prior to receiving the cytotoxic agent.
   105. The method of any one of embodiments 59-104, wherein the hematopoietic cells are hematopoietic stem cells.
   106. The method of embodiment 105, wherein the hematopoietic stem cells are from bone marrow cells, cord blood cells, or peripheral blood mononuclear cells (PBMCs).
   107. The method of embodiment 105 or embodiment 106, wherein the hematopoietic stem cells are CD34+/CD33−.
   108. The method of any one of embodiments 59-107, wherein the hematopoietic cells are autologous.
   109. The method of embodiment 108, wherein the method further comprises obtaining the autologous hematopoietic stem cells from the subject, optionally wherein the method further comprises genetically engineering the autologous stem cells to have reduced or eliminated expression of the CD33 antigen, and returning the genetically engineered hematopoietic stem cells to the subject.
   110. The method of any one of embodiments 59-107, wherein the hematopoietic cells are allogeneic.
   111. The method of embodiment 110, wherein the hematopoietic cells are allogeneic hematopoietic stem cells obtained from a donor having an HLA haplotype that matches with the HLA haplotype of the subject.
   112. The method of any one of embodiments 59-111, further comprising obtaining hematopoietic cells from a donor having an HLA haplotype that matches with the HLA haplotype of the subject.
   113. The method of any one of embodiments 59-112, further comprising preparing the hematopoietic cells by modifying an endogenous gene of the hematopoietic cells encoding the CD33 antigen.
   114. The method of embodiment 113, wherein the whole or a portion of the endogenous gene encoding the CD33 cell-surface antigen is deleted.
   115. The method of embodiment 113 or embodiment 114, wherein the whole or the portion of the endogenous gene is deleted using genome editing.
   116. The method of embodiment 115, wherein the genome editing involves a zinc finger nuclease (ZFN), a transcription activator-like effector-based nuclease (TALEN), or a CRISPR-Cas system.
   117. A method, comprising administering to a subject: an effective amount of a population of genetically modified hematopoietic cells, or descendants thereof, comprising a modified gene encoding CD33 that is engineered to have reduced or eliminated expression of a CD33 antigen, wherein the subject is receiving or has received an effective amount of a cytotoxic agent comprising an anti-CD33 antigen-binding domain.
   118. The method of embodiment 117, wherein the cytotoxic agent is an antibody-drug conjugate (ADC).
   119. The method of embodiment 118, wherein the ADC is gemtuzumab ozogamicin.
   120. The method of any one of embodiments 117-119, wherein the effective amount of the population of genetically engineered hematopoietic cells is about 10⁶ cells/kilogram body weight of the subject to about 10⁷ cells/kilogram body weight of the subject.
   121. The method of embodiment 120, wherein the effective amount of the population of genetically engineered hematopoietic cells is about 3.0×10⁶ cells/kilogram body weight of the subject.
   122. The method of any one of embodiments 117-121, wherein the effective amount of the cytotoxic agent is about 0.1 mg/m² body surface area of the subject to about 2.0 mg/m² body surface area of the subject.
   123. The method of embodiment 122, wherein the effective amount of the cytotoxic agent is about 0.1 mg/m², about 0.25 mg/m², about 0.5 mg/m², about 1.0 mg/m², or about 2.0 mg/m² body surface area of the subject.
   124. The method of embodiment 123, wherein the effective amount of the cytotoxic agent is about 2.0 mg/m² body surface area of the subject.
   125. The method of any one of embodiments 117-124, wherein the effective amount of the cytotoxic agent is administered in temporal proximity with the effective amount of the population of genetically engineered hematopoietic cells.
   126. The method of embodiment 125, wherein administering in temporal proximity comprises administering the population of genetically engineered hematopoietic cells and the cytotoxic agent within a single treatment regimen.
   127. The method of embodiment 125, wherein administering in temporal proximity comprises administering the population of genetically engineered hematopoietic cells and the cytotoxic agent simultaneously.
   128. The method of embodiment 125, wherein administering in temporal proximity comprises administering the population of genetically engineered hematopoietic cells and the cytotoxic agent concurrently.
   129. The method of embodiment 125, wherein administering in temporal proximity comprises administering the population of genetically engineered hematopoietic cells and the cytotoxic agent sequentially.
   130. The method of embodiment 125, wherein administering in temporal proximity comprises administering the population of genetically engineered hematopoietic cells within 120 days of administering the cytotoxic agent.
   131. The method of embodiment 125, wherein administering in temporal proximity comprises administering the population of genetically engineered hematopoietic cells within 90 days of administering the cytotoxic agent.
   132. The method of embodiment 125, wherein administering in temporal proximity comprises administering the population of genetically engineered hematopoietic cells within 60 days of administering the cytotoxic agent.
   133. The method of any one of embodiments 117-132, wherein the population of genetically engineered hematopoietic cells are administered prior to the cytotoxic agent.
   134. The method of any one of embodiments 117-133, wherein the population of genetically engineered hematopoietic cells are administered in a single treatment regimen.
   135. The method of any one of embodiments 117-134, wherein the population of genetically engineered hematopoietic cells and/or the cytotoxic agent are administered intravenously.
   136. The method of any one of embodiments 117-135, wherein the cytotoxic agent is administered in multiple doses of the effective amount every four weeks.
   137. The method of any one of embodiments 117-136, wherein the cytotoxic agent is administered in multiple doses of about 2.0 mg/m² every four weeks.
   138. The method of any one of embodiments 117-137, wherein the population of genetically engineered hematopoietic cells are thawed from a cryopreserved form prior to administration.
   139. The method of any one of embodiments 117-138, wherein the cytotoxic agent is reconstituted from a lyophilized form prior to administration.
   140. The method of any one of embodiments 117-139, wherein the subject has been preconditioned prior to administering the cytotoxic agent and/or the population of genetically engineered hematopoietic cells.
   141. The method of any one of embodiments 117-140, further comprising preconditioning the subject prior to administering the cytotoxic agent and/or the population of genetically engineered hematopoietic cells.
   142. The method of embodiment 140 or embodiment 141, wherein the preconditioning comprises administering one or more chemotherapeutic agents to the subject.
   143. The method of any one of embodiments 140-142, wherein the preconditioning comprises total body irradiation of the subject.
   144. The method of embodiment 142 or embodiment 143, wherein the chemotherapeutic agent is selected from the group consisting of busulfan, melphalan, fludarabine, cyclophosphamide, and thiotepa.
   145. The method of any one of embodiments 140-144, wherein the preconditioning comprises administering antibodies that bind human T cells, optionally wherein the antibodies comprise rabbit anti-thymocyte globulins (rATG).
   146. The method of any one of embodiments 117-145, wherein the subject has, or has been diagnosed with, a hematopoietic malignancy or a hematopoietic pre-malignant disease, and wherein the hematopoietic malignancy is characterized by the presence of CD33-positive malignant cells, or wherein the hematopoietic pre-malignant disease is characterized by the presence of CD33-positive pre-malignant cells.
   147. The method of any one of embodiments 117-146, wherein the subject has, or has been diagnosed with, CD33-positive acute myeloid leukemia.
   148. The method of any one of claims embodiments 117-146, wherein the subject has, or has been diagnosed with, CD33-positive myelodysplastic syndrome.
   149. The method of any one of embodiments 117-146, wherein the subject has, or has been diagnosed with, CD33-positive myelodysplastic syndrome and wherein the subject is at high risk of developing acute myeloid leukemia.
   150. The method of any one of embodiments 117-149, wherein the subject is naïve to chemotherapy and/or radiation therapy, optionally wherein the subject is naïve to any treatment aimed to address a hematopoietic malignancy or hematopoietic pre-malignant disease.
   151. The method of any one of embodiments 117-149, wherein the subject has previously received chemotherapy.
   152. The method of any one of embodiments 117-151, wherein the subject has previously received induction therapy.
   153. The method of any one of embodiments 117-152, wherein the subject has previously entered a complete hematological remission, optionally wherein the complete hematological remission is characterized by an incomplete recovery of peripheral counts.
   154. The method of any one of embodiments 117-153, wherein the subject has one or more risk factors associated with early leukemia relapse.
   155. The method of embodiment 154, wherein the one or more risk factors associated with early leukemia relapse are selected from the group consisting of: bone marrow in morphological complete remission with presence of intermediate or high-risk disease-related genetics; presence of minimal residual disease (MRD) post cyto-reductive therapy; bone marrow with persistent leukemia blasts post cyto-reductive therapy; and bone marrow blast count of about 10% or less with no circulating blasts.
   156. The method of any one of embodiments 117-155, wherein the subject does not have a homozygous dominant genotype for CD33 single nucleotide polymorphism (SNP) rs12459419.
   157. The method of any one of embodiments 117-156, wherein the subject does not have acute promyelocytic leukemia or chronic myeloid leukemia.
   158. The method of any one of embodiments 117-157, wherein the subject does not have a genetic translocation associated with acute promyelocytic leukemia or chronic myeloid leukemia, optionally wherein the genetic translocation is t(15; 17)(q22; q21) or t(9; 22)(q34; q11).
   159. The method of any one of embodiments 117-158, wherein the subject has not previously received a stem cell transplantation.
   160. The method of any one of embodiments 117-159, wherein the subject has not previously received the cytotoxic agent.
   161. The method of any one of embodiments 117-160, further comprising determining a percent donor chimerism and/or a level of CD33-negative myeloid hematopoiesis in a peripheral blood sample from the subject.
   162. The method of any one of embodiments 117-161, wherein the subject has a CD33-negative absolute neutrophil count (ANC) of at least 1000/dL prior to receiving the cytotoxic agent.
   163. The method of any one of embodiments 117-162, wherein the hematopoietic cells are hematopoietic stem cells.
   164. The method of embodiment 163, wherein the hematopoietic stem cells are from bone marrow cells, cord blood cells, or peripheral blood mononuclear cells (PBMCs).
   165. The method of embodiment 163 or embodiment 164, wherein the hematopoietic stem cells are CD34+/CD33−.
   166. The method of any one of embodiments 117-165, wherein the hematopoietic cells are autologous.
   167. The method of embodiment 166, wherein the method further comprises obtaining the autologous hematopoietic stem cells from the subject, optionally wherein the method further comprises genetically engineering the autologous stem cells to have reduced or eliminated expression of the CD33 antigen, and returning the genetically engineered hematopoietic stem cells to the subject.
   168. The method of any one of embodiments 117-165, wherein the hematopoietic cells are allogeneic.
   169. The method of embodiment 168, wherein the hematopoietic cells are allogeneic hematopoietic stem cells obtained from a donor having an HLA haplotype that matches with the HLA haplotype of the subject.
   170. The method of any one of embodiments 117-169, further comprising obtaining hematopoietic cells from a donor having an HLA haplotype that matches with the HLA haplotype of the subject.
   171. The method of any one of embodiments 117-170, further comprising preparing the hematopoietic cells by modifying an endogenous gene of the hematopoietic cells encoding the CD33 antigen.
   172. The method of embodiment 171, wherein the whole or a portion of the endogenous gene encoding the CD33 cell-surface antigen is deleted.
   173. The method of embodiment 171 or embodiment 172, wherein the whole or the portion of the endogenous gene is deleted using genome editing.
   174. The method of embodiment 173, wherein the genome editing involves a zinc finger nuclease (ZFN), a transcription activator-like effector-based nuclease (TALEN), or a CRISPR-Cas system.
   175. A composition comprising a population of genetically modified hematopoietic cells, or descendants thereof, comprising a modified gene encoding CD33 that is engineered to have reduced or eliminated expression of a CD33 antigen.
   176. The composition of embodiment 175, wherein the hematopoietic cells are hematopoietic stem cells from bone marrow cells, cord blood cells, or peripheral blood mononuclear cells (PBMCs).
   177. The composition of embodiment 176, wherein the hematopoietic stem cells are CD34+/CD33−.
   178. The composition of any one or more of embodiments 175-177, wherein the whole or a portion of an endogenous gene encoding the CD33 antigen is deleted.
   179. The composition of any one or more of embodiments 175-178, wherein the whole or the portion of the endogenous gene is deleted using genome editing.
   180. The composition of embodiment 179, wherein the genome editing carried out involves a zinc finger nuclease (ZFN), a transcription activator-like effector-based nuclease (TALEN), or a CRISPR-Cas system.
   181. The composition of embodiment 180, wherein the CRISPR-Cas system comprises a nucleic acid encoding a gRNA and an RNA-guided nuclease.
   182. The composition of embodiment 181, wherein the gRNA comprises a targeting domain comprising a sequence of any one of SEQ ID NOs: 9-15.
   183. A combination comprising the population of genetically modified hematopoietic cells of any one of embodiments 175-182, and a cytotoxic agent comprising an anti-CD33 antigen-binding domain.
   184. The combination of embodiment 183, wherein the cytotoxic agent is an antibody-drug conjugate (ADC).
   185. The combination of embodiment 184, wherein the ADC is gemtuzumab ozogamicin.
   185. A composition comprising a population of at least 1×10⁶ cells per milliliter (mL) in a medium, wherein the population of cells comprise genetically modified hematopoietic cells, or descendants thereof, comprising a modified gene encoding CD33 that is engineered to have reduced or eliminated expression of a CD33 antigen.
   186. The composition of embodiment 185, wherein the medium has a volume of about 45 mL.
   187. The composition of embodiment 185 or embodiment 186, wherein at least 50%, at least 60%, at least 70%, at least 80%, at least 90%, at least 95%, or at least 99% of the cells of population are genetically modified hematopoietic cells, or descendants thereof, having reduced or eliminated expression of a CD33 antigen.
   188. The composition of any one of embodiments 185-187, wherein the population comprises at least 2×10⁶ cells per mL, at least 3×10⁶ cells per mL, at least 4×10⁶ cells per mL, at least 5×10⁶ cells per mL, at least 6×10⁶ cells per mL, at least at least 7×10⁶ cells per mL, at least 8×10⁶ cells per mL, or at least 9×10⁶ cells per mL.
   189. The composition of any one of embodiments 185-188, wherein the medium is a cryopreservation medium comprising a cryoprotectant.
   190. The composition of embodiment 189, wherein the cryoprotectant comprises dimethylsulfoxide (DMSO) in an amount of about 10% (v/v).
   191. The composition of any one of embodiments 185-190, wherein the hematopoietic cells are CD34+/CD33−.
   192. The composition of any one of embodiments 185-191, wherein the whole or a portion of an endogenous gene encoding the CD33 antigen is deleted.
   193. The composition of any one of embodiments 185-192, wherein the whole or the portion of the endogenous gene is deleted using genome editing.
   194. The composition of embodiment 193, wherein the genome editing carried out involves a zinc finger nuclease (ZFN), a transcription activator-like effector-based nuclease (TALEN), or a CRISPR-Cas system.
   195. The composition of embodiment 194, wherein the CRISPR-Cas system comprises a nucleic acid encoding a gRNA and an RNA-guided nuclease.
   196. The composition of embodiment 195, wherein the gRNA comprises a targeting domain comprising a sequence of any one of SEQ ID NOs: 9-15.
   197. The composition of embodiment 195 or embodiment 196, wherein the compositions does not comprise a detectable level of the RNA-guided nuclease.
   198. The composition of any one of embodiments 185-197, wherein the composition is in a frozen state.
   199. A cryopreserved composition comprising the composition of any one of embodiments 185-198, wherein the composition has been subjected to a cryopreservation process.
   200. The cryopreserved composition of embodiment 199, wherein the cryopreservation process is controlled-rate freezing.

### EQUIVALENTS AND SCOPE

Those skilled in the art will recognize, or be able to ascertain using no more than routine experimentation, many equivalents of the exemplary embodiments described herein. The scope of the present disclosure is not intended to be limited to the above description.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, it is to be understood that every possible individual element or subgroup of the elements is also disclosed, and that any element or subgroup of elements can be removed from the group. It is also noted that the term “comprising” is intended to be open and permits the inclusion of additional elements, features, or steps. It should be understood that, in general, where an embodiment, is referred to as comprising particular elements, features, or steps, embodiments, that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. For purposes of brevity, all of the embodiments in which one or more elements, features, purposes, or aspects is excluded are not set forth explicitly herein. The disclosure contemplates all combinations of any one or more of the foregoing embodiments, as well as combinations with any one or more of the embodiments set forth in the detailed description and examples.

Although methods and materials similar or equivalent to those described herein can be used in the practice or testing of the present invention, suitable methods and materials are described herein. All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of May 23, 2019. When one gene or protein references a plurality of sequence accession numbers, all of the sequence variants are encompassed.

What is claimed is:

1. A method, comprising administering to a subject:
an effective amount of a population of genetically engineered hematopoietic cells, or descendants thereof, comprising a modified gene encoding CD33 that is engineered to have reduced or eliminated expression of a CD33 antigen; and
an effective amount of a cytotoxic agent comprising an anti-CD33 antigen-binding domain.

2. The method of claim 1, wherein the cytotoxic agent is an antibody-drug conjugate (ADC).

3. The method of claim 2, wherein the ADC is gemtuzumab ozogamicin.

4. The method of any one of claims 1-3, wherein the effective amount of the population of genetically engineered hematopoietic cells is about 10⁶ cells/kilogram body weight of the subject to about 10⁷ cells/kilogram body weight of the subject.

5. The method of claim 4, wherein the effective amount of the population of genetically engineered hematopoietic cells is about 3.0×10⁶ cells/kilogram body weight of the subject.

6. The method of any one of claims 1-5, wherein the effective amount of the cytotoxic agent is about 0.1 mg/m² body surface area of the subject to about 2.0 mg/m² body surface area of the subject.

7. The method of claim 6, wherein the effective amount of the cytotoxic agent is about 0.1 mg/m², about 0.25 mg/m², about 0.5 mg/m², about 1.0 mg/m², or about 2.0 mg/m² body surface area of the subject.

8. The method of claim 7, wherein the effective amount of the cytotoxic agent is about 2.0 mg/m² body surface area of the subject.

9. The method of any one of claims 1-8, wherein the population of genetically engineered hematopoietic cells and the cytotoxic agent are administered in temporal proximity.

10. The method of claim 9, wherein administering in temporal proximity comprises administering the population of genetically engineered hematopoietic cells and the cytotoxic agent within a single treatment regimen.

11. The method of claim 9, wherein administering in temporal proximity comprises administering the population of genetically engineered hematopoietic cells and the cytotoxic agent simultaneously.

12. The method of claim 9, wherein administering in temporal proximity comprises administering the population of genetically engineered hematopoietic cells and the cytotoxic agent concurrently.

13. The method of claim 9, wherein administering in temporal proximity comprises administering the population of genetically engineered hematopoietic cells and the cytotoxic agent sequentially.

14. The method of claim 9, wherein administering in temporal proximity comprises administering the population of genetically engineered hematopoietic cells within 120 days of administering the cytotoxic agent.

15. The method of claim 14, wherein administering in temporal proximity comprises administering the population of genetically engineered hematopoietic cells within 90 days of administering the cytotoxic agent.

16. The method of claim 15, wherein administering in temporal proximity comprises administering the population of genetically engineered hematopoietic cells within 60 days of administering the cytotoxic agent.

17. The method of any one of claims 1-9 or 12-16, wherein the population of genetically engineered hematopoietic cells are administered prior to the cytotoxic agent.

18. The method of any one of claims 1-17, wherein the population of genetically engineered hematopoietic cells are administered in a single treatment regimen.

19. The method of any one of claims 1-18, wherein the population of genetically engineered hematopoietic cells and/or the cytotoxic agent are administered intravenously.

20. The method of any one of claims 1-19, wherein the cytotoxic agent is administered in multiple doses of the effective amount every four weeks.

21. The method of any one of claims 1-20, wherein the cytotoxic agent is administered in multiple doses of about 2.0 mg/m² every four weeks.

22. The method of any one of claims 1-21, wherein the population of genetically engineered hematopoietic cells are thawed from a cryopreserved form prior to administration.

23. The method of any one of claims 1-22, wherein the cytotoxic agent is reconstituted from a lyophilized form prior to administration.

24. The method of any one of claims 1-23, wherein the subject has been preconditioned prior to administering the cytotoxic agent and/or the hematopoietic cells.

25. The method of any one of claims 1-24, further comprising preconditioning the subject prior to administering the cytotoxic agent and/or the hematopoietic cells.

26. The method of claim 24 or claim 25, wherein the preconditioning comprises administering one or more chemotherapeutic agents to the subject.

27. The method of any one of claims 24-26, wherein the preconditioning comprises total body irradiation of the subject.

28. The method of any one of claims 24-27, wherein the chemotherapeutic agent is selected from the group consisting of busulfan, melphalan, fludarabine, cyclophosphamide, and thiotepa.

29. The method of any one of claims 24-28, wherein the preconditioning comprises administering antibodies that bind human T cells, optionally wherein the antibodies comprise rabbit anti-thymocyte globulins (rATG).

30. The method of any one of claims 1-29, wherein the subject has, or has been diagnosed with, a hematopoietic malignancy or a hematopoietic pre-malignant disease, and wherein the hematopoietic malignancy is characterized by the presence of CD33-positive malignant cells, or wherein the hematopoietic pre-malignant disease is characterized by the presence of CD33-positive pre-malignant cells.

31. The method of any one of claims 1-30, wherein the subject has, or has been diagnosed with, CD33-positive acute myeloid leukemia.

32. The method of any one of claims 1-30, wherein the subject has, or has been diagnosed with, CD33-positive myelodysplastic syndrome.

33. The method of any one of claims 1-30, wherein the subject has, or has been diagnosed with, CD33-positive myelodysplastic syndrome and wherein the subject is at high risk of developing acute myeloid leukemia.

34. The method of any one of claims 1-33, wherein the subject is naïve to chemotherapy and/or radiation therapy, optionally wherein the subject is naïve to any treatment aimed to address a hematopoietic malignancy or hematopoietic pre-malignant disease.

35. The method of any one of claims 1-33, wherein the subject has previously received chemotherapy.

36. The method of any one of claims 1-35, wherein the subject has previously received induction therapy.

37. The method of any one of claims 1-36, wherein the subject has previously entered a complete hematological remission, optionally wherein the complete hematological remission is characterized by an incomplete recovery of peripheral counts.

38. The method of any one of claims 1-37, wherein the subject has one or more risk factors associated with early leukemia relapse.

39. The method of claim 38, wherein the one or more risk factors associated with early leukemia relapse are selected from the group consisting of: bone marrow in morphological complete remission with presence of intermediate or high-risk disease-related genetics; presence of minimal residual disease (MRD) post cyto-reductive therapy; bone marrow with persistent leukemia blasts post cyto-reductive therapy; and bone marrow blast count of about 10% or less with no circulating blasts.

40. The method of any one of claims 1-39, wherein the subject does not have a homozygous dominant genotype for CD33 single nucleotide polymorphism (SNP) rs12459419.

41. The method of any one of claims 1-40, wherein the subject does not have acute promyelocytic leukemia or chronic myeloid leukemia.

42. The method of any one of claims 1-41, wherein the subject does not have a genetic translocation associated with acute promyelocytic leukemia or chronic myeloid leukemia, optionally wherein the genetic translocation is t(15; 17)(q22; q21) or t(9; 22)(q34; q11).

43. The method of any one of claims 1-42, wherein the subject has not previously received a stem cell transplantation.

44. The method of any one of claims 1-43, wherein the subject has not previously received the cytotoxic agent.

45. The method of any one of claims 1-44, further comprising determining a percent donor chimerism and/or a level of CD33-negative myeloid hematopoiesis in a peripheral blood sample from the subject.

46. The method of any one of claims 1-45, wherein the subject has a CD33-negative absolute neutrophil count (ANC) of at least 1000/dL prior to receiving the cytotoxic agent.

47. The method of any one of claims 1-46, wherein the hematopoietic cells are hematopoietic stem cells.

48. The method of claim 47, wherein the hematopoietic stem cells are from bone marrow cells, cord blood cells, or peripheral blood mononuclear cells (PBMCs).

49. The method of claim 47 or 48, wherein the hematopoietic stem cells are CD34+/CD33−.

50. The method of any one of claims 1-49, wherein the hematopoietic cells are autologous.

51. The method of claim 50, wherein the method further comprises obtaining the autologous hematopoietic stem cells from the subject, optionally wherein the method further comprises genetically engineering the autologous stem cells to have reduced or eliminated expression of the CD33 antigen, and returning the genetically engineered hematopoietic stem cells to the subject.

52. The method of any one of claims 1-49, wherein the hematopoietic cells are allogeneic.

53. The method of claim 52, wherein the hematopoietic cells are allogeneic hematopoietic stem cells obtained from a donor having an HLA haplotype that matches with the HLA haplotype of the subject.

54. The method of any one of claims 1-53, further comprising obtaining hematopoietic cells from a donor having an HLA haplotype that matches with the HLA haplotype of the subject.

55. The method of any one of claims 1-54, further comprising preparing the hematopoietic cells by modifying an endogenous gene of the hematopoietic cells encoding the CD33 antigen.

56. The method of claim 55, wherein the whole or a portion of the endogenous gene encoding the CD33 cell-surface antigen is deleted.

57. The method of claim 55 or 56, wherein the whole or the portion of the endogenous gene is deleted using genome editing.

58. The method of claim 57, wherein the genome editing involves a zinc finger nuclease (ZFN), a transcription activator-like effector-based nuclease (TALEN), or a CRISPR-Cas system.

59. A method, comprising administering to a subject:
an effective amount of a cytotoxic agent comprising an anti-CD33 antigen-binding domain,
wherein the subject is receiving or has received an effective amount of a population of genetically modified hematopoietic cells, or descendants thereof, comprising a modified gene encoding CD33 that is engineered to have reduced or eliminated expression of a CD33 agent.

60. The method of claim 59, wherein the cytotoxic agent is an antibody-drug conjugate (ADC).

61. The method of claim 60, wherein the ADC is gemtuzumab ozogamicin.

62. The method of any one of claims 59-61, wherein the effective amount of the population of genetically engineered hematopoietic cells is about 10⁶ cells/kilogram body weight of the subject to about 10⁷ cells/kilogram body weight of the subject.

63. The method of claim 62, wherein the effective amount of the population of genetically engineered hematopoietic cells is about 3.0×10⁶ cells/kilogram body weight of the subject.

64. The method of any one of claims 59-63, wherein the effective amount of the cytotoxic agent is about 0.1 mg/m² body surface area of the subject to about 2.0 mg/m² body surface area of the subject.

65. The method of claim 64, wherein the effective amount of the cytotoxic agent is about 0.1 mg/m², about 0.25 mg/m², about 0.5 mg/m², about 1.0 mg/m², or about 2.0 mg/m² body surface area of the subject.

66. The method of claim 65, wherein the effective amount of the cytotoxic agent is about 2.0 mg/m² body surface area of the subject.

67. The method of any one of claims 59-66, wherein the effective amount of the cytotoxic agent is administered in temporal proximity with the effective amount of the population of genetically engineered hematopoietic cells.

68. The method of claim 67, wherein administering in temporal proximity comprises administering the population of genetically engineered hematopoietic cells and the cytotoxic agent within a single treatment regimen.

69. The method of claim 67, wherein administering in temporal proximity comprises administering the population of genetically engineered hematopoietic cells and the cytotoxic agent simultaneously.

70. The method of claim 67, wherein administering in temporal proximity comprises administering the population of genetically engineered hematopoietic cells and the cytotoxic agent concurrently.

71. The method of claim 67, wherein administering in temporal proximity comprises administering the population of genetically engineered hematopoietic cells and the cytotoxic agent sequentially.

72. The method of claim 67, wherein administering in temporal proximity comprises administering the cytotoxic agent within 120 days of administering the population of genetically engineered hematopoietic cells.

73. The method of claim 67, wherein administering in temporal proximity comprises administering the cytotoxic agent within 90 days of administering the population of genetically engineered hematopoietic cells.

74. The method of claim 67, wherein administering in temporal proximity comprises administering the cytotoxic agent within 60 days of administering the hematopoietic cells. population of genetically engineered hematopoietic cells

75. The method of any one of claims 59-74, wherein the population of genetically engineered hematopoietic cells are administered prior to the cytotoxic agent.

76. The method of any one of claims 59-75, wherein the population of genetically engineered hematopoietic cells are administered in a single treatment regimen.

77. The method of any one of claims 59-76, wherein the population of genetically engineered hematopoietic cells and/or the cytotoxic agent are administered intravenously.

78. The method of any one of claims 59-77, wherein the cytotoxic agent is administered in multiple doses of the effective amount every four weeks.

79. The method of any one of claims 59-78, wherein the cytotoxic agent is administered in multiple doses of about 2.0 mg/m² every four weeks.

80. The method of any one of claims 59-79, wherein the population of genetically engineered hematopoietic cells are thawed from a cryopreserved form prior to administration.

81. The method of any one of claims 59-80, wherein the cytotoxic agent is reconstituted from a lyophilized form prior to administration.

82. The method of any one of claims 59-81, wherein the subject has been preconditioned prior to administering the cytotoxic agent and/or the hematopoietic cells.

83. The method of any one of claims 59-82 further comprising preconditioning the subject prior to administering the cytotoxic agent and/or the population of genetically engineered hematopoietic cells.

84. The method of claim 82 or claim 83, wherein the preconditioning comprises administering one or more chemotherapeutic agents to the subject.

85. The method of any one of claims 82-84, wherein the preconditioning comprises total body irradiation of the subject.

86. The method of any one of claims 82-85, wherein the chemotherapeutic agent is selected from the group consisting of busulfan, melphalan, fludarabine, cyclophosphamide, and thiotepa.

87. The method of any one of claims 82-86, wherein the preconditioning comprises administering antibodies that bind human T cells, optionally wherein the antibodies comprise rabbit anti-thymocyte globulins (rATG).

88. The method of any one of claims 59-87, wherein the subject has, or has been diagnosed with, a hematopoietic malignancy or a hematopoietic pre-malignant disease, and wherein the hematopoietic malignancy is characterized by the presence of CD33-positive malignant cells, or wherein the hematopoietic pre-malignant disease is characterized by the presence of CD33-positive pre-malignant cells.

89. The method of any one of claims 59-88, wherein the subject has, or has been diagnosed with, CD33-positive acute myeloid leukemia.

90. The method of any one of claims 59-88, wherein the subject has, or has been diagnosed with, CD33-positive myelodysplastic syndrome.

91. The method of any one of claims 59-88, wherein the subject has, or has been diagnosed with, CD33-positive myelodysplastic syndrome and wherein the subject is at high risk of developing acute myeloid leukemia.

92. The method of any one of claims 59-91, wherein the subject is naïve to chemotherapy and/or radiation therapy, optionally wherein the subject is naïve to any treatment aimed to address a hematopoietic malignancy or hematopoietic pre-malignant disease.

93. The method of any one of claims 59-91, wherein the subject has previously received chemotherapy.

94. The method of any one of claims 59-93, wherein the subject has previously received induction therapy.

95. The method of any one of claims 59-94, wherein the subject has previously entered a complete hematological remission, optionally wherein the complete hematological remission is characterized by an incomplete recovery of peripheral counts.

96. The method of any one of claims 59-95, wherein the subject has one or more risk factors associated with early leukemia relapse.

97. The method of claim 96, wherein the one or more risk factors associated with early leukemia relapse are selected from the group consisting of: bone marrow in morphological complete remission with presence of intermediate or high-risk disease-related genetics; presence of minimal residual disease (MRD) post cyto-reductive therapy; bone marrow with persistent leukemia blasts post cyto-reductive therapy; and bone marrow blast count of about 10% or less with no circulating blasts.

98. The method of any one of claims 59-97, wherein the subject does not have a homozygous dominant genotype for CD33 single nucleotide polymorphism (SNP) rs12459419.

99. The method of any one of claims 59-98, wherein the subject does not have acute promyelocytic leukemia or chronic myeloid leukemia.

100. The method of any one of claims 59-99, wherein the subject does not have a genetic translocation associated with acute promyelocytic leukemia or chronic myeloid leukemia, optionally wherein the genetic translocation is t(15; 17)(q22; q21) or t(9; 22)(q34; q11).

101. The method of any one of claims 59-100, wherein the subject has not previously received a stem cell transplantation.

102. The method of any one of claims 59-101, wherein the subject has not previously received the cytotoxic agent.

103. The method of any one of claims 59-102, further comprising determining a percent donor chimerism and/or a level of CD33-negative myeloid hematopoiesis in a peripheral blood sample from the subject.

104. The method of any one of claims 59-103, wherein the subject has a CD33-negative absolute neutrophil count (ANC) of at least 1000/dL prior to receiving the cytotoxic agent.

105. The method of any one of claims 59-104, wherein the hematopoietic cells are hematopoietic stem cells.

106. The method of claim 105, wherein the hematopoietic stem cells are from bone marrow cells, cord blood cells, or peripheral blood mononuclear cells (PBMCs).

107. The method of claim 105 or claim 106, wherein the hematopoietic stem cells are CD34+/CD33−.

108. The method of any one of claims 59-107, wherein the hematopoietic cells are autologous.

109. The method of claim 108, wherein the method further comprises obtaining the autologous hematopoietic stem cells from the subject, optionally wherein the method further comprises genetically engineering the autologous stem cells to have reduced or eliminated expression of the CD33 antigen, and returning the genetically engineered hematopoietic stem cells to the subject.

110. The method of any one of claims 59-107, wherein the hematopoietic cells are allogeneic.

111. The method of claim 110, wherein the hematopoietic cells are allogeneic hematopoietic stem cells obtained from a donor having an HLA haplotype that matches with the HLA haplotype of the subject.

112. The method of any one of claims 59-111, further comprising obtaining hematopoietic cells from a donor having an HLA haplotype that matches with the HLA haplotype of the subject.

113. The method of any one of claims 59-112, further comprising preparing the hematopoietic cells by modifying an endogenous gene of the hematopoietic cells encoding the CD33 antigen.

114. The method of claim 113, wherein the whole or a portion of the endogenous gene encoding the CD33 cell-surface antigen is deleted.

115. The method of claim 113 or claim 114, wherein the whole or the portion of the endogenous gene is deleted using genome editing.

116. The method of claim 115, wherein the genome editing involves a zinc finger nuclease (ZFN), a transcription activator-like effector-based nuclease (TALEN), or a CRISPR-Cas system.

117. A method, comprising administering to a subject:
an effective amount of a population of genetically modified hematopoietic cells, or descendants thereof, comprising a modified gene encoding CD33 that is engineered to have reduced or eliminated expression of a CD33 antigen,
wherein the subject is receiving or has received an effective amount of a cytotoxic agent comprising an anti-CD33 antigen-binding domain.

118. The method of claim 117, wherein the cytotoxic agent is an antibody-drug conjugate (ADC).

119. The method of claim 118, wherein the ADC is gemtuzumab ozogamicin.

120. The method of any one of claims 117-119, wherein the effective amount of the population of genetically engineered hematopoietic cells is about 10⁶ cells/kilogram body weight of the subject to about 10⁷ cells/kilogram body weight of the subject.

121. The method of claim 120, wherein the effective amount of the population of genetically engineered hematopoietic cells is about 3.0×10⁶ cells/kilogram body weight of the subject.

122. The method of any one of claims 117-121, wherein the effective amount of the cytotoxic agent is about 0.1 mg/m² body surface area of the subject to about 2.0 mg/m² body surface area of the subject.

123. The method of claim 122, wherein the effective amount of the cytotoxic agent is about 0.1 mg/m², about 0.25 mg/m², about 0.5 mg/m², about 1.0 mg/m², or about 2.0 mg/m² body surface area of the subject.

124. The method of claim 123, wherein the effective amount of the cytotoxic agent is about 2.0 mg/m² body surface area of the subject.

125. The method of any one of claims 117-124, wherein the effective amount of the cytotoxic agent is administered in temporal proximity with the effective amount of the population of genetically engineered hematopoietic cells.

126. The method of claim 125, wherein administering in temporal proximity comprises administering the population of genetically engineered hematopoietic cells and the cytotoxic agent within a single treatment regimen.

127. The method of claim 125, wherein administering in temporal proximity comprises administering the population of genetically engineered hematopoietic cells and the cytotoxic agent simultaneously.

128. The method of claim 125, wherein administering in temporal proximity comprises administering the population of genetically engineered hematopoietic cells and the cytotoxic agent concurrently.

129. The method of claim 125, wherein administering in temporal proximity comprises administering the population of genetically engineered hematopoietic cells and the cytotoxic agent sequentially.

130. The method of claim 125, wherein administering in temporal proximity comprises administering the population of genetically engineered hematopoietic cells within 120 days of administering the cytotoxic agent.

131. The method of claim 125, wherein administering in temporal proximity comprises administering the population of genetically engineered hematopoietic cells within 90 days of administering the cytotoxic agent.

132. The method of claim 125, wherein administering in temporal proximity comprises administering the population of genetically engineered hematopoietic cells within 60 days of administering the cytotoxic agent.

133. The method of any one of claims 117-132, wherein the population of genetically engineered hematopoietic cells are administered prior to the cytotoxic agent.

134. The method of any one of claims 117-133, wherein the population of genetically engineered hematopoietic cells are administered in a single treatment regimen.

135. The method of any one of claims 117-134, wherein the population of genetically engineered hematopoietic cells and/or the cytotoxic agent are administered intravenously.

136. The method of any one of claims 117-135, wherein the cytotoxic agent is administered in multiple doses of the effective amount every four weeks.

137. The method of any one of claims 117-136, wherein the cytotoxic agent is administered in multiple doses of about 2.0 mg/m² every four weeks.

138. The method of any one of claims 117-137, wherein the population of genetically engineered hematopoietic cells are thawed from a cryopreserved form prior to administration.

139. The method of any one of claims 117-138, wherein the cytotoxic agent is reconstituted from a lyophilized form prior to administration.

140. The method of any one of claims 117-139, wherein the subject has been preconditioned prior to administering the cytotoxic agent and/or the population of genetically engineered hematopoietic cells.

141. The method of any one of claims 117-140, further comprising preconditioning the subject prior to administering the cytotoxic agent and/or the population of genetically engineered hematopoietic cells.

142. The method of claim 140 or claim 141, wherein the preconditioning comprises administering one or more chemotherapeutic agents to the subject.

143. The method of any one of claims 140-142, wherein the preconditioning comprises total body irradiation of the subject.

144. The method of any one of claim 142 or claim 143, wherein the chemotherapeutic agent is selected from the group consisting of busulfan, melphalan, fludarabine, cyclophosphamide, and thiotepa.

145. The method of any one of claims 140-144, wherein the preconditioning comprises administering antibodies that bind human T cells, optionally wherein the antibodies comprise rabbit anti-thymocyte globulins (rATG).

146. The method of any one of claims 117-145, wherein the subject has, or has been diagnosed with, a hematopoietic malignancy or a hematopoietic pre-malignant disease, and wherein the hematopoietic malignancy is characterized by the presence of CD33-positive malignant cells, or wherein the hematopoietic pre-malignant disease is characterized by the presence of CD33-positive pre-malignant cells.

147. The method of any one of claims 117-146, wherein the subject has, or has been diagnosed with, CD33-positive acute myeloid leukemia.

148. The method of any one of claims 117-146, wherein the subject has, or has been diagnosed with, CD33-positive myelodysplastic syndrome.

149. The method of any one of claims 117-146, wherein the subject has, or has been diagnosed with, CD33-positive myelodysplastic syndrome and wherein the subject is at high risk of developing acute myeloid leukemia.

150. The method of any one of claims 117-149, wherein the subject is naïve to chemotherapy and/or radiation therapy, optionally wherein the subject is naïve to any treatment aimed to address a hematopoietic malignancy or hematopoietic pre-malignant disease.

151. The method of any one of claims 117-149, wherein the subject has previously received chemotherapy.

152. The method of any one of claims 117-151, wherein the subject has previously received induction therapy.

153. The method of any one of claims 117-152, wherein the subject has previously entered a complete hematological remission, optionally wherein the complete hematological remission is characterized by an incomplete recovery of peripheral counts.

154. The method of any one of claims 117-153, wherein the subject has one or more risk factors associated with early leukemia relapse.

155. The method of claim 154, wherein the one or more risk factors associated with early leukemia relapse are selected from the group consisting of: bone marrow in morphological complete remission with presence of intermediate or high-risk disease-related genetics; presence of minimal residual disease (MRD) post cyto-reductive therapy; bone marrow with persistent leukemia blasts post cyto-reductive therapy; and bone marrow blast count of about 10% or less with no circulating blasts.

156. The method of any one of claims 117-155, wherein the subject does not have a homozygous dominant genotype for CD33 single nucleotide polymorphism (SNP) rs12459419.

157. The method of any one of claims 117-156, wherein the subject does not have acute promyelocytic leukemia or chronic myeloid leukemia.

158. The method of any one of claims 117-157, wherein the subject does not have a genetic translocation associated with acute promyelocytic leukemia or chronic myeloid leukemia, optionally wherein the genetic translocation is t(15; 17)(q22; q21) or t(9; 22)(q34; q11).

159. The method of any one of claims 117-158, wherein the subject has not previously received a stem cell transplantation.

160. The method of any one of claims 117-159, wherein the subject has not previously received the cytotoxic agent.

161. The method of any one of claims 117-160, further comprising determining a percent donor chimerism and/or a level of CD33-negative myeloid hematopoiesis in a peripheral blood sample from the subject.

162. The method of any one of claims 117-161, wherein the subject has a CD33-negative absolute neutrophil count (ANC) of at least 1000/dL prior to receiving the cytotoxic agent.

163. The method of any one of claims 117-162, wherein the hematopoietic cells are hematopoietic stem cells.

164. The method of claim 163, wherein the hematopoietic stem cells are from bone marrow cells, cord blood cells, or peripheral blood mononuclear cells (PBMCs).

165. The method of claim 163 or claim 164, wherein the hematopoietic stem cells are CD34+/CD33−.

166. The method of any one of claims 117-165, wherein the hematopoietic cells are autologous.

167. The method of claim 166, wherein the method further comprises obtaining the autologous hematopoietic stem cells from the subject, optionally wherein the method further comprises genetically engineering the autologous stem cells to have reduced or eliminated expression of the CD33 antigen, and returning the genetically engineered hematopoietic stem cells to the subject.

168. The method of any one of claims 117-165, wherein the hematopoietic cells are allogeneic.

169. The method of claim 168, wherein the hematopoietic cells are allogeneic hematopoietic stem cells obtained from a donor having an HLA haplotype that matches with the HLA haplotype of the subject.

170. The method of any one of claims 117-169, further comprising obtaining hematopoietic cells from a donor having an HLA haplotype that matches with the HLA haplotype of the subject.

171. The method of any one of claims 117-170, further comprising preparing the hematopoietic cells by modifying an endogenous gene of the hematopoietic cells encoding the CD33 antigen.

172. The method of claim 171, wherein the whole or a portion of the endogenous gene encoding the CD33 cell-surface antigen is deleted.

173. The method of claim 171 or claim 172, wherein the whole or the portion of the endogenous gene is deleted using genome editing.

174. The method of claim 173, wherein the genome editing involves a zinc finger nuclease (ZFN), a transcription activator-like effector-based nuclease (TALEN), or a CRISPR-Cas system.

175. A composition comprising a population of genetically modified hematopoietic cells, or descendants thereof, comprising a modified gene encoding CD33 that is engineered to have reduced or eliminated expression of a CD33 antigen.

176. The composition of claim 175, wherein the hematopoietic cells are hematopoietic stem cells from bone marrow cells, cord blood cells, or peripheral blood mononuclear cells (PBMCs).

177. The composition of claim 176, wherein the hematopoietic stem cells are CD34+/CD33−.

178. The composition of any one or more of claims 175-177, wherein the whole or a portion of an endogenous gene encoding the CD33 antigen is deleted.

179. The composition of any one or more of claims 175-178, wherein the whole or the portion of the endogenous gene is deleted using genome editing.

180. The composition of claim 179, wherein the genome editing carried out involves a zinc finger nuclease (ZFN), a transcription activator-like effector-based nuclease (TALEN), or a CRISPR-Cas system.

181. The composition of claim 180, wherein the CRISPR-Cas system comprises a nucleic acid encoding a gRNA and an RNA-guided nuclease.

182. The composition of claim 181, wherein the gRNA comprises a targeting domain comprising a sequence of any one of SEQ ID NOs: 9-15.

183. A combination comprising the population of genetically modified hematopoietic cells of any one of claims 175-182, and a cytotoxic agent comprising an anti-CD33 antigen-binding domain.

184. The combination of claim 183, wherein the cytotoxic agent is an antibody-drug conjugate (ADC).

185. The combination of claim 184, wherein the ADC is gemtuzumab ozogamicin.

185. A composition comprising a population of at least 1×10⁶ cells per milliliter (mL) in a medium, wherein the population of cells comprise genetically modified hematopoietic cells, or descendants thereof, comprising a modified gene encoding CD33 that is engineered to have reduced or eliminated expression of a CD33 antigen.

186. The composition of claim 185, wherein the medium has a volume of about 45 mL.

187. The composition of claim 185 or claim 186, wherein at least 50%, at least 60%, at least 70%, at least 80%, at least 90%, at least 95%, or at least 99% of the cells of population are genetically modified hematopoietic cells, or descendants thereof, having reduced or eliminated expression of a CD33 antigen.

188. The composition of any one of claims 185-187, wherein the population comprises at least 2×10⁶ cells per mL, at least 3×10⁶ cells per mL, at least 4×10⁶ cells per mL, at least 5×10⁶ cells per mL, at least 6×10⁶ cells per mL, at least at least 7×10⁶ cells per mL, at least 8×10⁶ cells per mL, or at least 9×10⁶ cells per mL.

189. The composition of any one of claims 185-188, wherein the medium is a cryopreservation medium comprising a cryoprotectant.

190. The composition of claim 189, wherein the cryoprotectant comprises dimethylsulfoxide (DMSO) in an amount of about 10% (v/v).

191. The composition of any one of claims 185-190, wherein the hematopoietic cells are CD34+/CD33−.

192. The composition of any one of claims 185-191, wherein the whole or a portion of an endogenous gene encoding the CD33 antigen is deleted.

193. The composition of any one of claims 185-192, wherein the whole or the portion of the endogenous gene is deleted using genome editing.

194. The composition of claim 193, wherein the genome editing carried out involves a zinc finger nuclease (ZFN), a transcription activator-like effector-based nuclease (TALEN), or a CRISPR-Cas system.

195. The composition of claim 194, wherein the CRISPR-Cas system comprises a nucleic acid encoding a gRNA and an RNA-guided nuclease.

196. The composition of claim 195, wherein the gRNA comprises a targeting domain comprising a sequence of any one of SEQ ID NOs: 9-15.

197. The composition of claim 195 or claim 196, wherein the compositions does not comprise a detectable level of the RNA-guided nuclease.

198. The composition of any one of claims 185-197, wherein the composition is in a frozen state.

199. A cryopreserved composition comprising the composition of any one of 185-198, wherein the composition has been subjected to a cryopreservation process.

200. The cryopreserved composition of claim 199, wherein the cryopreservation process is controlled-rate freezing.


SEQUENCE LISTING (66 sequences)

SEQ ID NO: 1 - DNA, 20 bp, Homo sapiens
ccccaggactactcactcct

SEQ ID NO: 2 - DNA, 20 bp, Homo sapiens
accgaggagtgagtagtcct

SEQ ID NO: 3 - DNA, 20 bp, Homo sapiens
ggtgggggcagctgacaacc

SEQ ID NO: 4 - DNA, 20 bp, Homo sapiens
cggtgctcataatcacccca

SEQ ID NO: 5 - DNA, 20 bp, Homo sapiens
aggagtgagtagtcctgggg

SEQ ID NO: 6 - DNA, 20 bp, Homo sapiens
aggactactcactcctcggt

SEQ ID NO: 7 - DNA, 20 bp, Homo sapiens
ggttgtcagctgcccccacc

SEQ ID NO: 8 - DNA, 20 bp, Homo sapiens
tggggtgattatgagcaccg

SEQ ID NO: 9 - RNA, 20 nt, Artificial Sequence
  Synthetic
ccccaggacuacucacuccu

SEQ ID NO: 10 - RNA, 20 nt, Artificial Sequence
  Synthetic
accgaggagugaguaguccu

SEQ ID NO: 11 - RNA, 20 nt, Artificial Sequence
  Synthetic
ggugggggcagcugacaacc

SEQ ID NO: 12 - RNA, 20 nt, Artificial Sequence
  Synthetic
cggugcucauaaucacccca

SEQ ID NO: 13 - DNA, 20 bp, Homo sapiens
cctcactagacttgacccac

SEQ ID NO: 14 - DNA, 20 bp, Homo sapiens
gtgggtcaagtctagtgagg

SEQ ID NO: 15 - RNA, 20 nt, Artificial Sequence
  Synthetic
ccucacuagacuugacccac

SEQ ID NO: 16 - DNA, 1095 bp, Homo sapiens
atgccgctgctgctactgctgcccctgctgtgggcaggggccctggctatggatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacagacttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctcaacgtcacctatgttccacagaacccaacaactggtatctttccaggagatggctcagggaaacaagagaccagagcaggagtggttcatggggccattggaggagctggtgttacagccctgctcgctctttgtctctgcctcatcttcttcatagtgaagacccacaggaggaaagcagccaggacagcagtgggcaggaatgacacccaccctaccacagggtcagcctccccgaaacaccagaagaagtccaagttacatggccccactgaaacctcaagctgttcaggtgccgcccctactgtggagatggatgaggagctgcattatgcttccctcaactttcatgggatgaatccttccaaggacacctccaccgaatactcagaggtcaggacccagtga

SEQ ID NO: 17 - DNA, 279 bp, Homo sapiens
acttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctcaacgtcacct

SEQ ID NO: 18 - RNA, 80 nt, Artificial Sequence
  Synthetic
guuuuagagcuagaaauagcaaguuaaaauaaggcuaguccguuaucaacuugaaaaaguggcaccgagucggugcuuuu

SEQ ID NO: 19 - RNA, 100 nt, Artificial Sequence
  Synthetic
ccccaggacuacucacuccuguuuuagagcuagaaauagcaaguuaaaauaaggcuaguccguuaucaacuugaaaaaguggcaccgagucggugcuuuu

SEQ ID NO: 20 - RNA, 100 nt, Artificial Sequence
  Synthetic
accgaggagugaguaguccuguuuuagagcuagaaauagcaaguuaaaauaaggcuaguccguuaucaacuugaaaaaguggcaccgagucggugcuuuu

SEQ ID NO: 21 - RNA, 100 nt, Artificial Sequence
  Synthetic
ggugggggcagcugacaaccguuuuagagcuagaaauagcaaguuaaaauaaggcuaguccguuaucaacuugaaaaaguggcaccgagucggugcuuuu

SEQ ID NO: 22 - RNA, 100 nt, Artificial Sequence
  Synthetic
cggugcucauaaucaccccaguuuuagagcuagaaauagcaaguuaaaauaaggcuaguccguuaucaacuugaaaaaguggcaccgagucggugcuuuu

SEQ ID NO: 23 - RNA, 100 nt, Artificial Sequence
  Synthetic
ccucacuagacuugacccacguuuuagagcuagaaauagcaaguuaaaauaaggcuaguccguuaucaacuugaaaaaguggcaccgagucggugcuuuu

SEQ ID NO: 24 - RNA, 100 nt, Artificial Sequence; identical to SEQ ID NO: 19
  Synthetic
  modified_base (2)..(4): may be modified by 2′-O-methyl-3′- phosphorothioate
  modified_base (98)..(100): may be modified by 2′-O-methyl-3′- phosphorothioate

SEQ ID NO: 25 - RNA, 100 nt, Artificial Sequence; identical to SEQ ID NO: 23
  Synthetic
  modified_base (2)..(4): may be modified by 2′-O-methyl-3′- phosphorothioate
  modified_base (98)..(100): may be modified by 2′-O-methyl-3′- phosphorothioate

SEQ ID NO: 26 - protein, 443 aa, Artificial Sequence
  Synthetic
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 218 aa, Artificial Sequence
  Synthetic
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - DNA, 23 bp, Artificial Sequence
  Synthetic
acctgtcaggtgaagttcgctgg

SEQ ID NO: 29 - DNA, 23 bp, Artificial Sequence
  Synthetic
tggccgggttctagagtgccagg

SEQ ID NO: 30 - DNA, 23 bp, Artificial Sequence
  Synthetic
ggccgggttctagagtgccaggg

SEQ ID NO: 31 - DNA, 23 bp, Artificial Sequence
  Synthetic
caccgaggagtgagtagtcctgg

SEQ ID NO: 32 - DNA, 23 bp, Artificial Sequence
  Synthetic
tccagcgaacttcacctgacagg

SEQ ID NO: 33 - DNA, 20 bp, Artificial Sequence
  Synthetic
gctgtggggagaggggttgt

SEQ ID NO: 34 - DNA, 20 bp, Artificial Sequence
  Synthetic
ctgtggggagaggggttgtc

SEQ ID NO: 35 - DNA, 20 bp, Artificial Sequence
  Synthetic
tggggaaacgagggtcagct

SEQ ID NO: 36 - DNA, 20 bp, Artificial Sequence
  Synthetic
gggcccctgtggggaaacga

SEQ ID NO: 37 - DNA, 20 bp, Artificial Sequence
  Synthetic
agggcccctgtggggaaacg

SEQ ID NO: 38 - DNA, 20 bp, Artificial Sequence
  Synthetic
gctgaccctcgtttccccac

SEQ ID NO: 39 - DNA, 20 bp, Artificial Sequence
  Synthetic
ctgaccctcgtttccccaca

SEQ ID NO: 40 - DNA, 20 bp, Artificial Sequence
  Synthetic
tgaccctcgtttccccacag

SEQ ID NO: 41 - DNA, 20 bp, Artificial Sequence
  Synthetic
ccatagccagggcccctgtg

SEQ ID NO: 42 - DNA, 20 bp, Artificial Sequence
  Synthetic
gcatgtgacaggtgaggcac

SEQ ID NO: 43 - DNA, 20 bp, Artificial Sequence
  Synthetic
tgaggcacaggcttcagaag

SEQ ID NO: 44 - DNA, 20 bp, Artificial Sequence
  Synthetic
aggcttcagaagtggccgca

SEQ ID NO: 45 - DNA, 20 bp, Artificial Sequence
  Synthetic
ggcttcagaagtggccgcaa

SEQ ID NO: 46 - DNA, 20 bp, Artificial Sequence
  Synthetic
gtacccatgaacttcccttg

SEQ ID NO: 47 - DNA, 20 bp, Artificial Sequence
  Synthetic
gtggccgcaagggaagttca

SEQ ID NO: 48 - DNA, 20 bp, Artificial Sequence
  Synthetic
tggccgcaagggaagttcat

SEQ ID NO: 49 - DNA, 20 bp, Artificial Sequence
  Synthetic
ggaagttcatgggtactgca

SEQ ID NO: 50 - DNA, 20 bp, Artificial Sequence
  Synthetic
ttcatgggtactgcagggca

SEQ ID NO: 51 - DNA, 20 bp, Artificial Sequence
  Synthetic
ctaaacccctcccagtacca

SEQ ID NO: 52 - DNA, 20 bp, Artificial Sequence
  Synthetic
cactcacctgcccacagcag

SEQ ID NO: 53 - DNA, 20 bp, Artificial Sequence
  Synthetic
ccctgctgtgggcaggtgag

SEQ ID NO: 54 - DNA, 20 bp, Artificial Sequence
  Synthetic
tgggcaggtgagtggctgtg

SEQ ID NO: 55 - DNA, 20 bp, Artificial Sequence
  Synthetic
ggtgagtggctgtggggaga

SEQ ID NO: 56 - DNA, 20 bp, Artificial Sequence
  Synthetic
gtgagtggctgtggggagag

SEQ ID NO: 57 - DNA, 20 bp, Artificial Sequence
  Synthetic
tccatagccagggcccctgt

SEQ ID NO: 58 - DNA, 20 bp, Artificial Sequence
  Synthetic
ccatagccagggcccctgtg

SEQ ID NO: 59 - DNA, 20 bp, Artificial Sequence
  Synthetic
tggggaaacgagggtcagct

SEQ ID NO: 60 - DNA, 20 bp, Artificial Sequence
  Synthetic
gggcccctgtggggaaacga

SEQ ID NO: 61 - DNA, 23 bp, Artificial Sequence
  Synthetic
agaaatttggatccatagccagg

SEQ ID NO: 62 - DNA, 23 bp, Artificial Sequence
  Synthetic
atccctggcactctagaacccgg

SEQ ID NO: 63 - DNA, 23 bp, Artificial Sequence
  Synthetic
cctcactagacttgacccacagg

SEQ ID NO: 64 - DNA, 20 bp, Artificial Sequence
  Synthetic
atccatagccagggcccctg

SEQ ID NO: 65 - DNA, 20 bp, Artificial Sequence
  Synthetic
tcgtttccccacaggggccc

SEQ ID NO: 66 - DNA, 20 bp, Artificial Sequence
  Synthetic
tggctatggatccaaatttc